(19) United States
(12) Patent Application Publication
(10) Pub. No.: US 2006/0140972 A1
(43) Pub. Date: Jun. 29, 2006

(54) Staphylococcus saprophyticus nucleic acids and polypeptides

(75) Inventors: Richard A. Alm, Ashland, MA (US); Paul Manning, Berlin, MA (US); Kathleen MacCormack, North Chelmsford, MA (US); Robert E. McLaughlin, Hudson, MA (US)

Correspondence Address: ASTRAZENECA R&D BOSTON, 35 GATEHOUSE DRIVE, WALTHAM, MA, 02451-1215, US

(73) Assignee: AstraZeneca AB

(21) Appl. No.: 11/021,837

(22) Filed: Dec. 23, 2004

Related U.S. Application Data
(60) Provisional application No. 60/533,534, filed on Dec. 31, 2003.
(60) Provisional application No. 60/600,680, filed on Aug. 11, 2004.

(51) Int. Cl. C12Q 1/68; C07H 21/04; C12P 21/06; A61K 39/02; C12N 1/21; C07K 14/315

(52) U.S. Cl. 424/190.1; 435/6; 435/69.1; 435/252.3; 435/320.1; 530/350; 536/23.7

(57) ABSTRACT

Isolated nucleic acid molecules which encode proteins from Staphylococcus saprophyticus are described. The invention also provides antisense nucleic acid molecules, recombinant expression vectors containing nucleic acid molecules, and host cells into which the expression vectors have been introduced. The invention still further provides isolated proteins, mutated proteins, fusion proteins, antigenic peptides and methods for the treatment, prevention or detection of an S. saprophyticus-associated disease or disorder. Furthermore, the nucleic acid molecules and polypeptide molecules of the invention may be used for the identification of agents which modulate S. saprophyticus activity, e.g., a small molecules.

### CROSS-REFERENCE TO RELATED APPLICATIONS

This application is related to U.S. Provisional Application No. 60/533,534 filed Dec. 31, 2003 and U.S. Provisional Application No. 60/600,680, filed Aug. 11, 2004, which are incorporated herein by reference in its entirety.

### FIELD OF THE INVENTION

The invention relates to isolated nucleic acids and polypeptides derived from Staphylococcus saprophyticus that can be used as molecular targets for diagnostics, prophylaxis and treatment of pathological conditions, as well as materials and methods for the diagnosis, prevention, and treatment of pathological conditions resulting from bacterial infection. In particular, in these and in other regards, the invention relates to novel polynucleotides that encode polypeptides, as set forth in FIG. 1.

### BACKGROUND OF THE INVENTION

Coagulase-negative staphylococci (CoNS) are commensal organisms of human skin and urogenital mucosa and are the most frequently isolated bacteria from bloodstream infections in intensive care patients. They are major etiological agents of nosocomial bacteremia and can also colonize a variety of medical devices and a diagnostic challenge is to distinguish clinically significant CoNS from contaminant strains. Urinary tract infections are the second most common infectious presentations in community practice and Staphylococcus saprophyticus is the next most frequently encountered agent which causes acute urinary tract infections after Escherichia coli. While E. coli remains the predominant pathogen responsible for acute community-acquired uncomplicated urinary tract infections (approximately 80%), other uropathogens are becoming increasingly important. Since the late 1970s, S. saprophyticus has been recognized as a significant pathogen, responsible for 7% to 30% of acute uncomplicated urinary tract infections (Stamm et al., N Engl J Med. 329:1328-1334, 1993; Meers et al. J Clin Pathol 28:270-273, 1975; Gillespie et al., J Clin Pathol 31:348-350, 1978; Wallmark et al., J Infect Dis 138:791-797, 1978; Latham et al., JAMA 250:3063-3066, 1983; Jordan et al., J Infect Dis 142:510-515, 1980). S. saprophyticus is often isolated from the urine of female outpatients presenting with symptoms of acute urinary infections (Jordan et al., 1980 ibid; Svanborg et al., Infect Dis Clin N Am 11:513-529, 1997) and these symptoms can not be differentiated from infections caused by E. coli. The etiology of uncomplicated urinary tract infections has remained stable over the last quarter of a century, although, as in other community-acquired disease, there has been a perceptible increase in antimicrobial resistance in the pathogens that cause these infections making them more difficult to treat.

The etiology of complicated urinary tract infections is influenced by host factors that underlie the infection, such as age, diabetes, or catheterization. Consequently, these complicated infections have a more diverse etiology than their uncomplicated counterparts, with Staphylococcal species playing a less important role. In addition, infections caused by coagulase negative staphylococci (such as S. saprophyticus) are commonly associated with infections after instrumentation of the urinary tract, especially in pediatric populations (Schlager, Pediatr Drugs 3:219-227, 2001). There is some evidence that S. saprophyticus is the causative agent of chronic bacterial prostatitis in men (Bergman et al., 5:241-245, 1989), as well as the etiological agent of sexually transmitted urethritis (Goldenring,6:417-418, 1986). S. saprophyticus can be differentiated from other urinary coagulase-negative staphylococci (i.e. S. epidermidis) by several characteristics, including novobiocin resistance, aerobic growth requirements, carbohydrate utilization and urease production (Jordan et al., 1980 ibid). Classification of S. saprophyticus can be done using culture-based identification systems (Janda et al., J Clin Microbiol 32:2056-2059, 1994; von Baum et al., Eur J Clin Microbiol Infect Dis 17:849-852, 1998) or by a rapid PCR assay (Martineau et al., J Clin Microbiol 38:3280-3284, 2000), although correct diagnoses can still be challenging.

S. saprophyticus is proposed to adhere to uroepithelial cells by producing a number of surface-associated proteins, most notably Ssp, Sdrl and a hemagglutinin (Aas) (Gatermann et al., Zentralbl Bakteriol 278:258-274, 1993; Hell et al., Mol Microbiol 29:871-881, 1998; Gatermann et al., Infect Immun 60:1055-1060, 1992; Gatermann et al., Infect Immun 62:4556-4563, 1994; Sakinc et al., Int J Med Microbiol 291(suppl 32): V56, 2001). The hemagglutinin is responsible for binding to extracellular matrix proteins (e.g. fibronectin) and appears to be the major adhesion molecule and represents a new class of staphylococcal adhesions, while the newly identified Sdrl also binds extracellular matrix proteins. Ssp is a surface-associated fibrillar protein thought to be involved in the interaction of S. saprophyticus with uroepithelial cells. S. saprophyticus also produces a urease which contributes to cystopathogenicity and tissue invasiveness by inducing severe damage to the bladder tissue (Gatermann Infect Immun 57:110-116, 1989; Gatermann Infect Immun 57:2998-3002, 1989). The urease catalyzes the hydrolysis of urea in urine and causes the release of ammonia and CO₂. As a result, the urinary pH is elevated, which can eventually lead to the formation of bladder or kidney stones.

Management of uncomplicated urinary tract infections has been quite successful as the spectrum of organisms causing the infections is quite small, and the susceptibility of these organisms to antimicrobial agents has been predictable. As a result, empirical therapy with trimethoprim-sulfamethoxazole has been the standard approach for uncomplicated infections. However, resistance rates to this therapy now approach 20% in some countries and this has necessitated a change in therapy and fluoroquinolones are being used more commonly. As fluoroquinolone resistance rates increase, the concern is that management of uncomplicated urinary tract infections will become more difficult.

The genome size of S. saprophyticus is unknown, although it is estimated to be approximately 2.4 Mb based on the genome sizes of other Staphylococcus spp. There have been few molecular genetic studies on this increasingly important pathogen, as evidenced by the public GenBank database currently only having approximately 20 kb (0.85% of predicted genome size) of non-redundant DNA sequence specific to an isolate of S. saprophyticus.

### SUMMARY OF THE INVENTION

The invention provides novel nucleic acid molecules and polypeptides which have a variety of uses. The nucleic acid and polypeptide molecules of the invention can be used in a variety of methods, including for example, screening assays, diagnostic assays, methods of treatment (e.g., prophylactic and therapeutic), production of recombinant proteins, and generation of probes, primers, antibodies and antisense molecules. As described herein, an S. saprophyticus nucleic acid as detailed in FIG. 1 encodes a polypeptide having one or more of the activities as set forth in FIG. 1.

In an exemplary method, the isolated nucleic acid molecules or encoded polypeptides of the invention (or agents derived therefrom, for example, nucleic acid fragments, peptide fragments, peptidomimetics, antibodies etc.), can be used, for example, to screen for a candidate test compound or agent, e.g., a small molecule, which has a stimulatory or inhibitory effect on S. saprophyticus expression or S. saprophyticus activity, to screen for drugs or compounds which modulate S. saprophyticus activity, e.g., a small molecule, as well as to treat or prevent S. saprophyticus-associated diseases and disorders, preferably, urinary tract infections. The S. saprophyticus nucleic acid molecules of the present invention can be used to express S. saprophyticus polypeptides (e.g., via a recombinant expression vector in a host cell), to detect S. saprophyticus RNA (e.g., in a biological sample) or a genetic alteration in an S. saprophyticus gene, and to generate S. saprophyticus probes and primers to specifically identify S. saprophyticus or identify genes from other related bacterial species. In addition, the anti-S. saprophyticus antibodies of the invention can be used to detect and isolate S. saprophyticus polypeptides and to modulate S. saprophyticus polypeptide activity.

In one aspect, the invention features isolated nucleic acid molecules including nucleotide sequences within SEQ ID NO:1-57 as described in FIG. 1, which are predicted to encode particular polypeptides. The invention includes nucleotide sequences that are substantially identical (at least 50% homologous) to the nucleotide sequences which are detailed in FIG. 1. For example, with respect to SEQ ID NO: 1, the invention includes a nucleic acid sequence having nucleotides 835-1164 of SEQ ID NO:1, or fragments thereof. In another example, the invention includes a nucleic acid sequence having nucleotides 1205-1535 of SEQ ID NO: 1, or fragments thereof. The invention also features isolated nucleic acid molecules at least 15 contiguous nucleotides to the nucleotide sequence set forth in FIG. 1.

The invention further includes polypeptide sequences (at least 50% homologous) encoded by the nucleic acid sequences set forth in FIG. 1. For example, as set forth in FIG. 1, the nucleic acid sequence having nucleotides 4653-6590 of SEQ ID NO:48 encodes a polypeptide of a DNA gyrase subunit B. The amino acid sequence of this predicted protein, SEQ ID NO: 58, can be readily determined by one skilled in the art. The present invention also features nucleic acid molecules which encode fragments, for example, biologically active or antigenic fragments of the polypeptides of the present invention. In still other aspects, the invention features nucleic acid molecules that hybridize under stringent conditions to the isolated nucleic acid molecules described herein.

In another embodiment, the present invention provides vectors including the isolated nucleic acid molecules described herein (e.g., S. saprophyticus encoding nucleic acid molecules). Such vectors can optionally include nucleotide sequences encoding heterologous polypeptides. Also featured are host cells including such vectors (e.g., host cells including vectors suitable for producing S. saprophyticus nucleic acid molecules and polypeptides). A method of producing a polypeptide consisting of culturing the host cell in an appropriate culture medium to produce a polypeptide as described herein.

In a further aspect, the present invention features methods for detecting S. saprophyticus polypeptides and/or S. saprophyticus nucleic acid molecules, such methods featuring, for example, a probe, primer or antibody described herein. Also featured are kits for the detection of S. saprophyticus polypeptides and/or S. saprophyticus nucleic acid molecules. In addition, compositions, including vaccine compositions, and methods for the protection or treatment of infection by S. saprophyticus are within the scope of this invention.

In yet another aspect, the invention features methods for identifying compounds which bind to and/or modulate the activity of an S. saprophyticus polypeptide or S. saprophyticus nucleic acid molecule as described herein. Also featured are methods for modulating an S. saprophyticus nucleic acid or polypeptide activity. In a related aspect, the present invention provides a diagnostic method of assessing whether a patient is afflicted with a S. saprophyticus-associated disease or disorder. Moreover, the invention provides a diagnostic method of assessing whether a patient has an S. saprophyticus-associated disease or disorder.

Other features and advantages of the invention will be apparent from the following detailed description and claims.

### BRIEF DESCRIPTION OF THE FIGURES

FIG. 1 is a table detailing the nucleic acid sequences which encode particular polypeptides encompassed by the present invention.

### DETAILED DESCRIPTION OF THE INVENTION

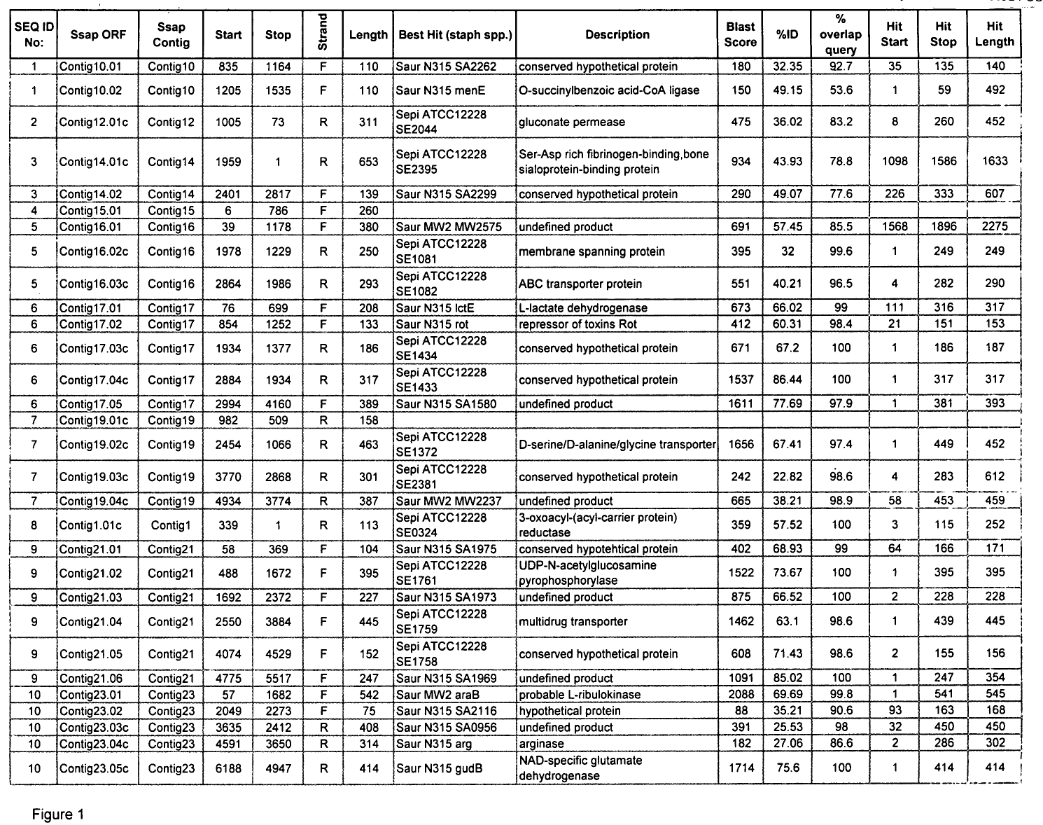

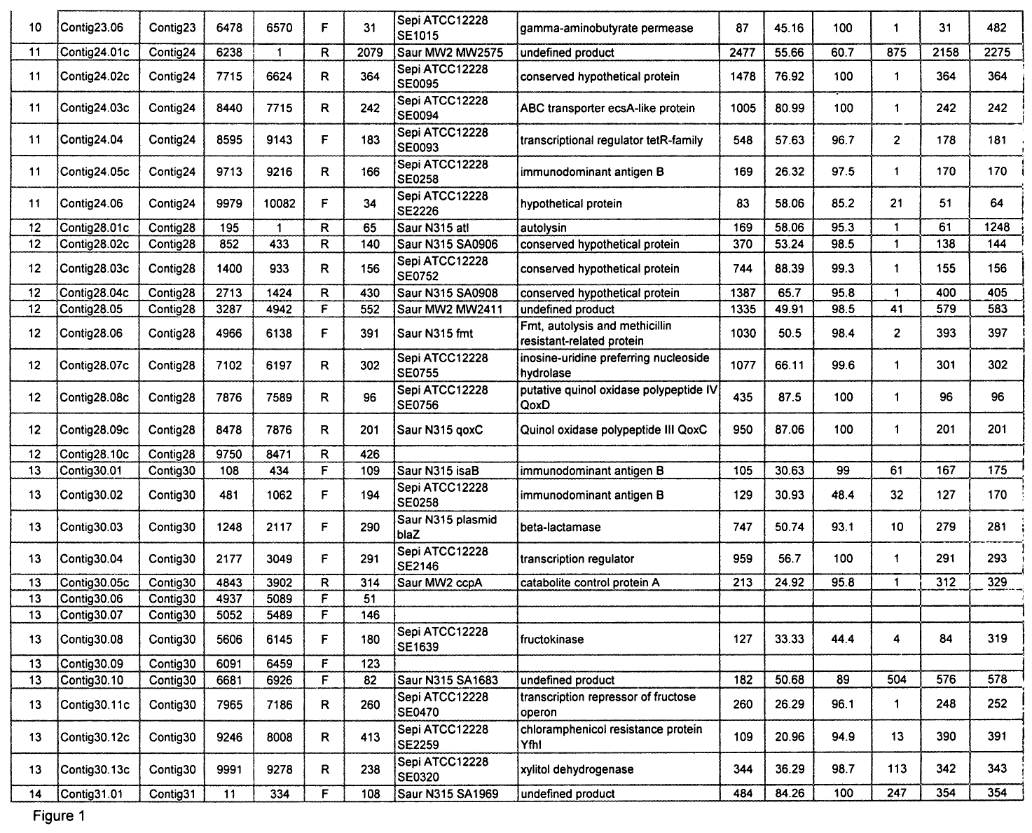

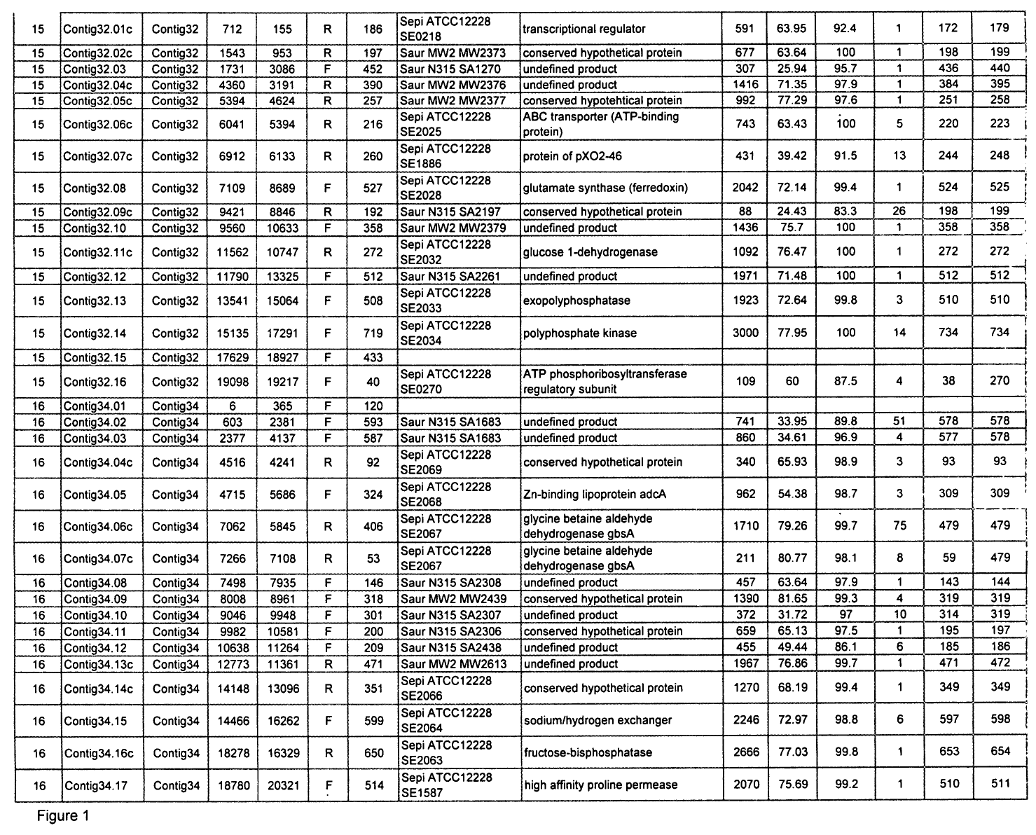

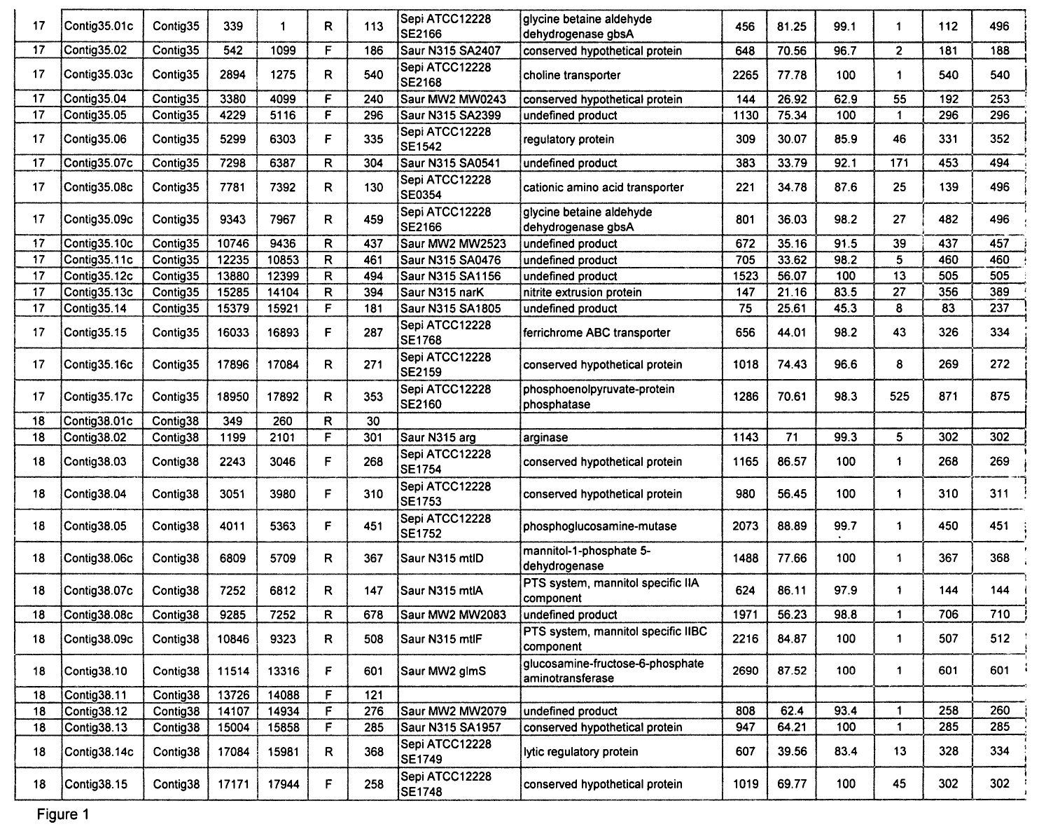

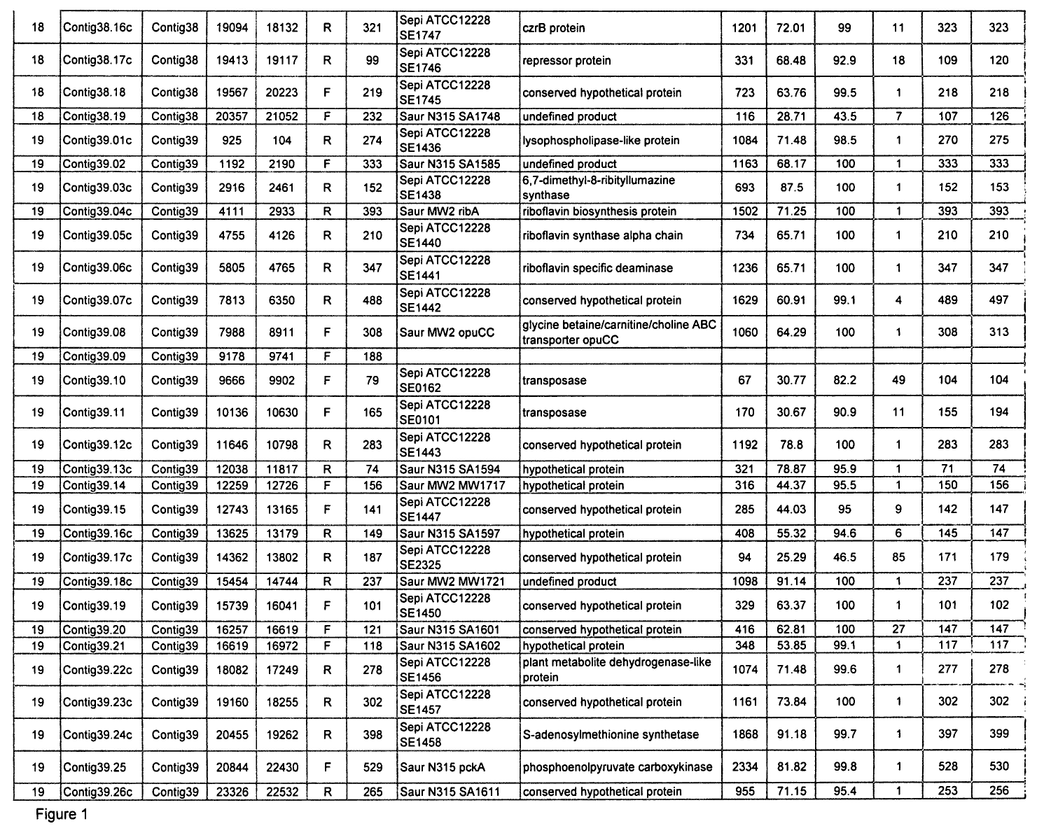

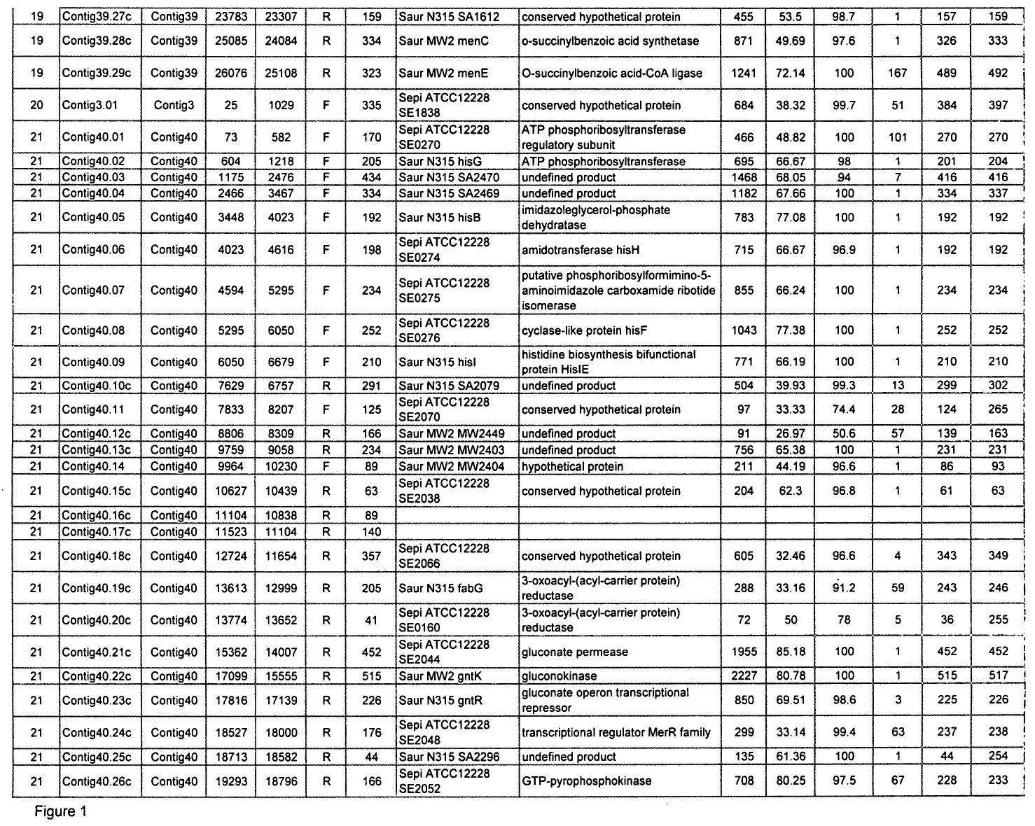

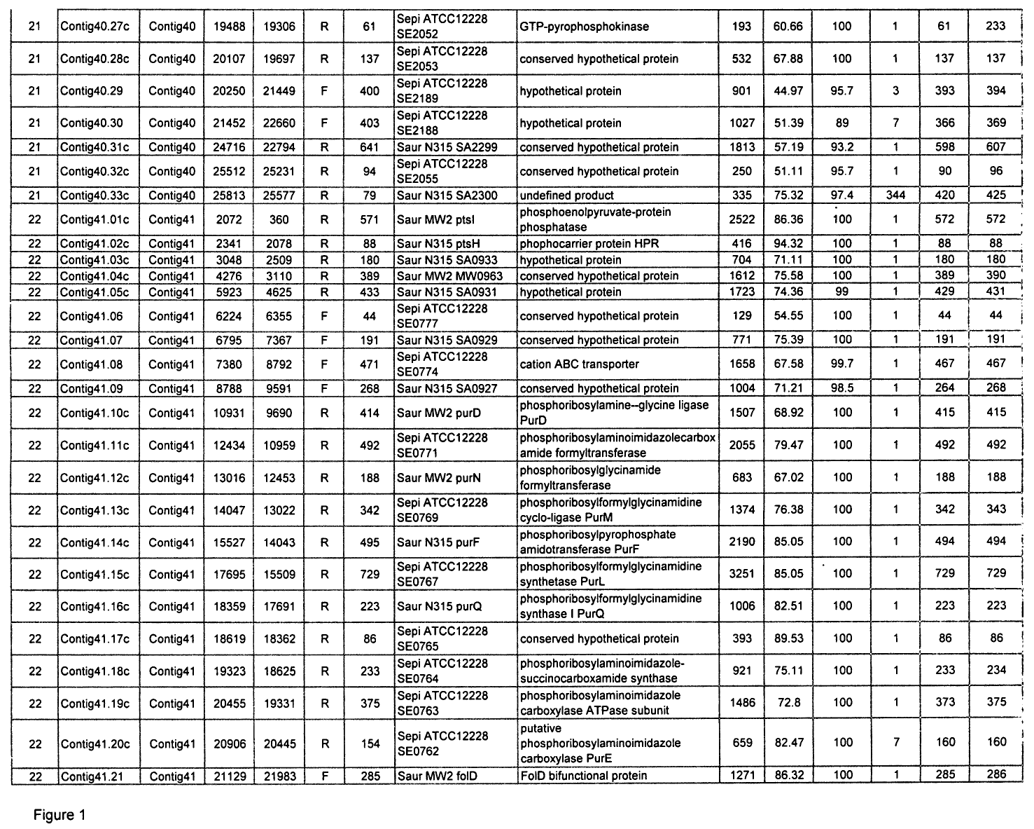

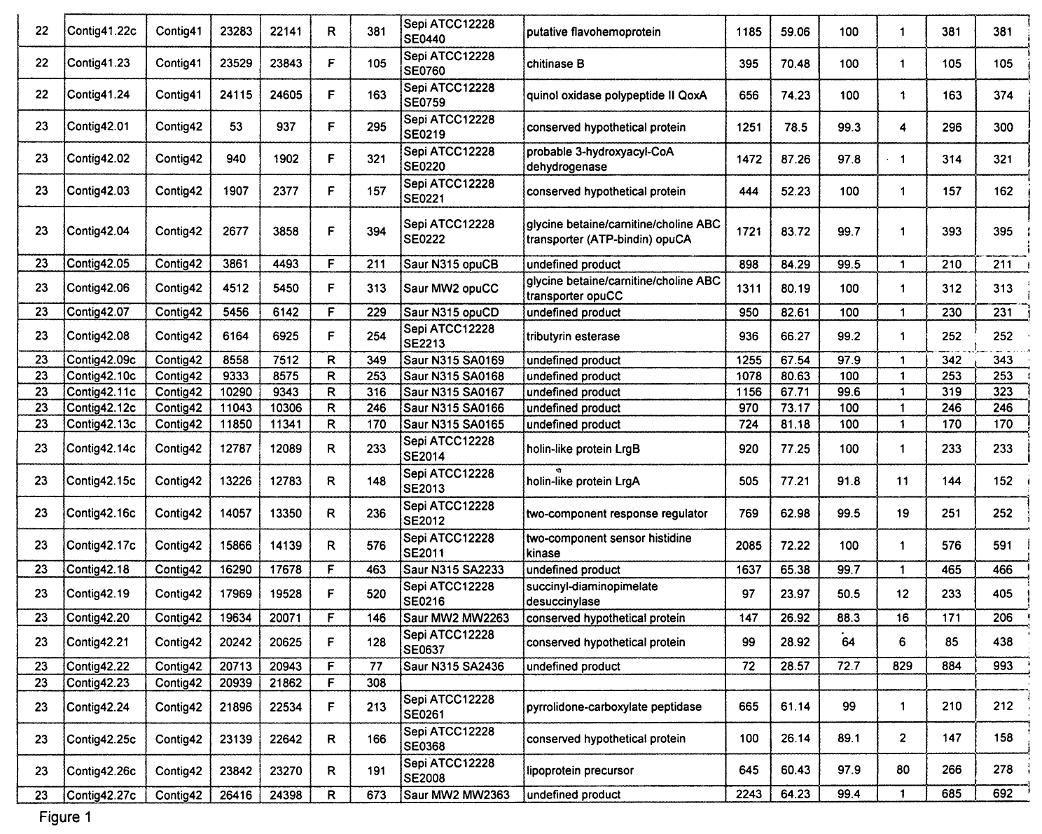

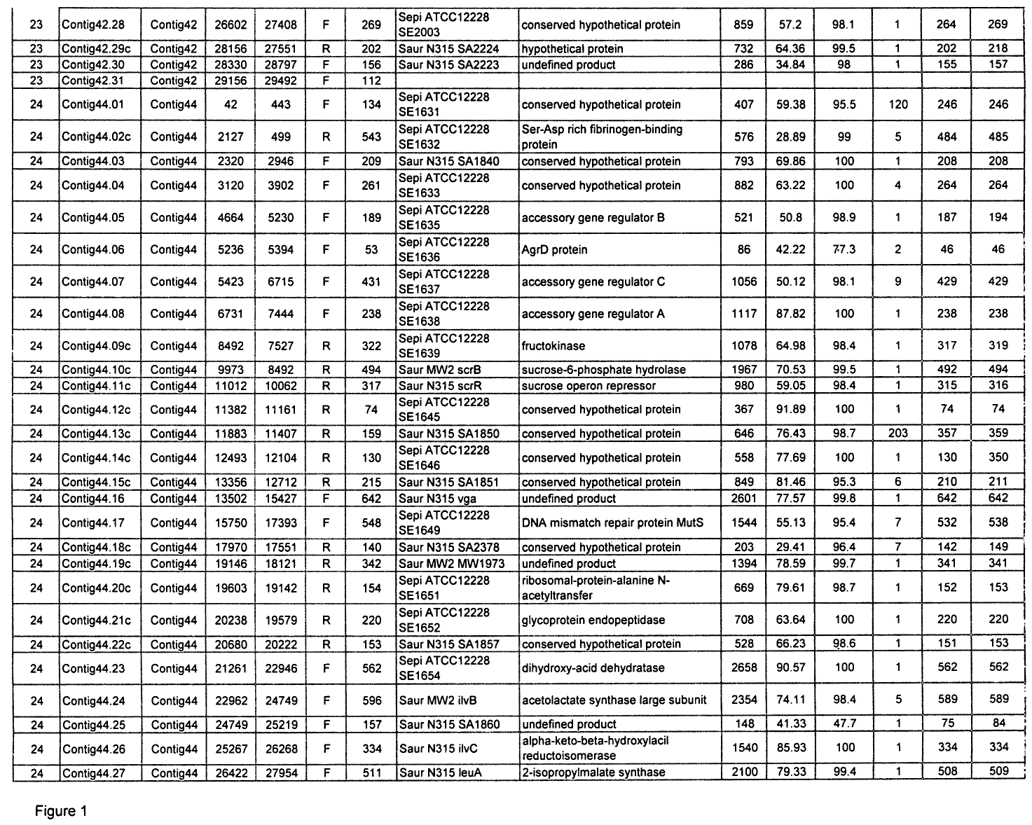

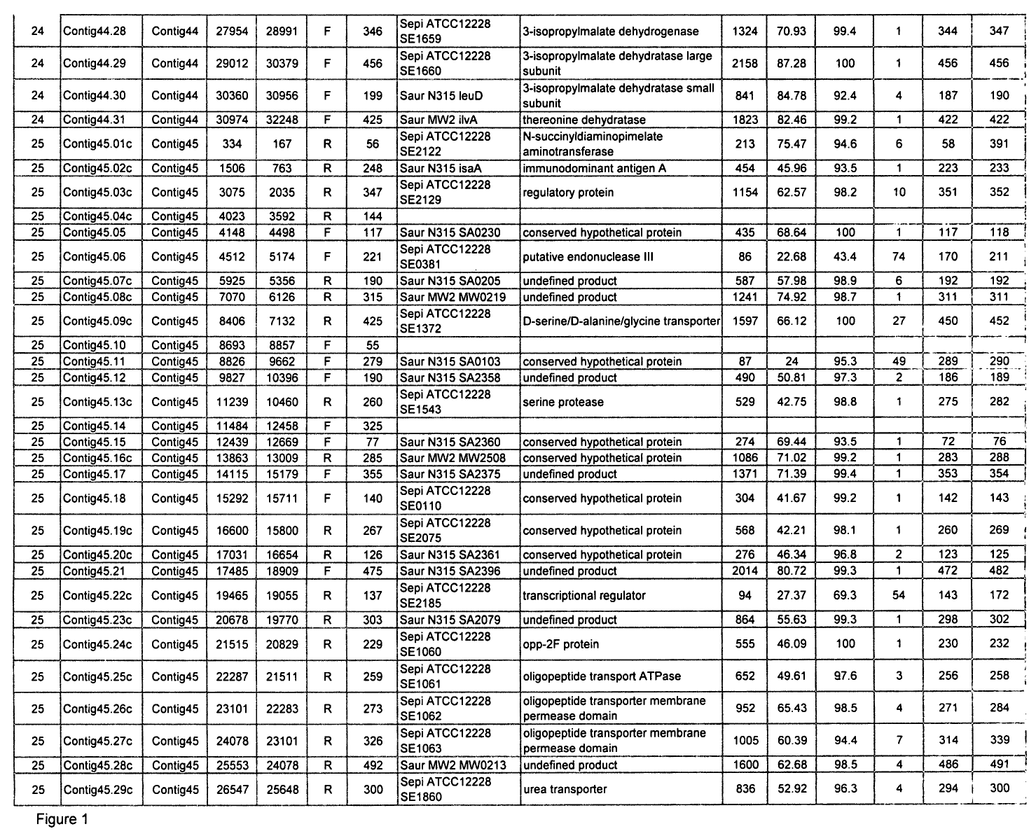

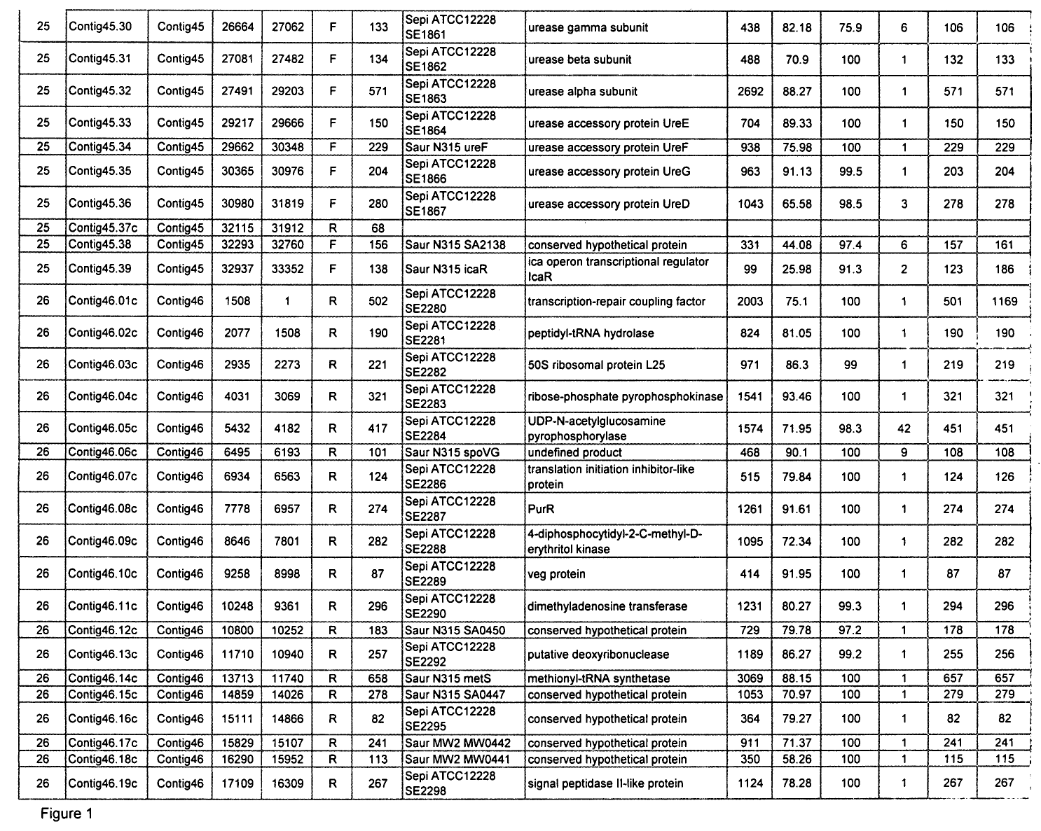

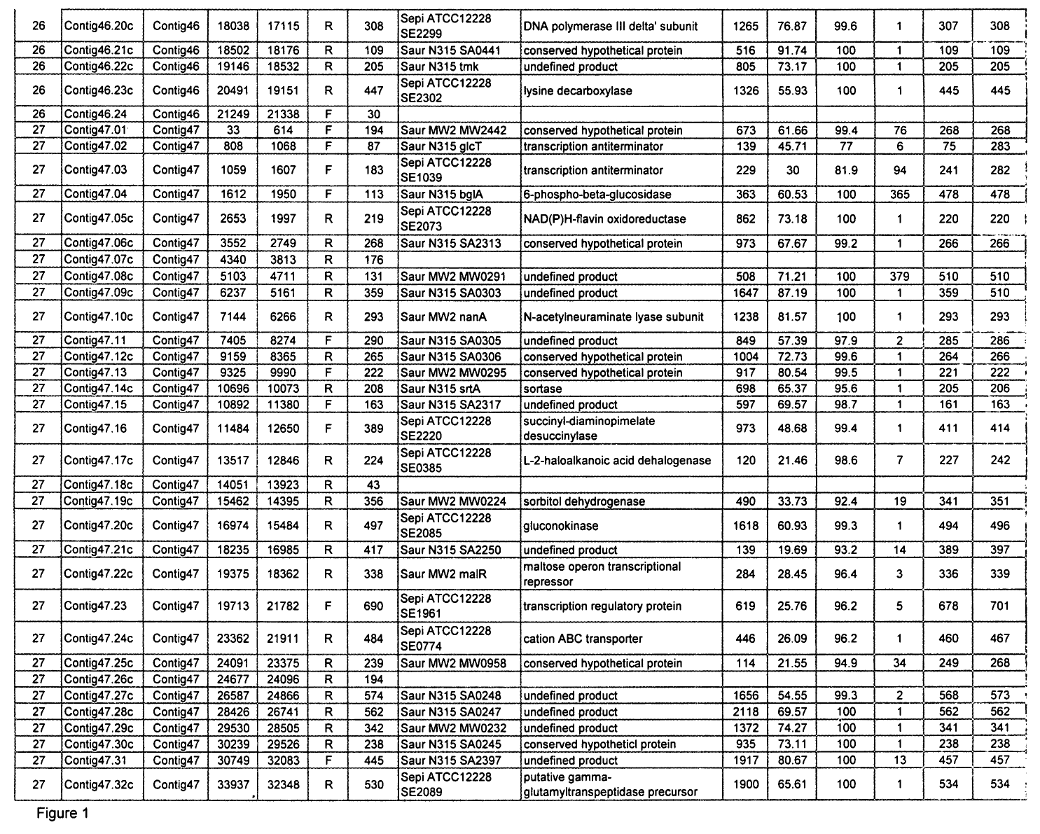

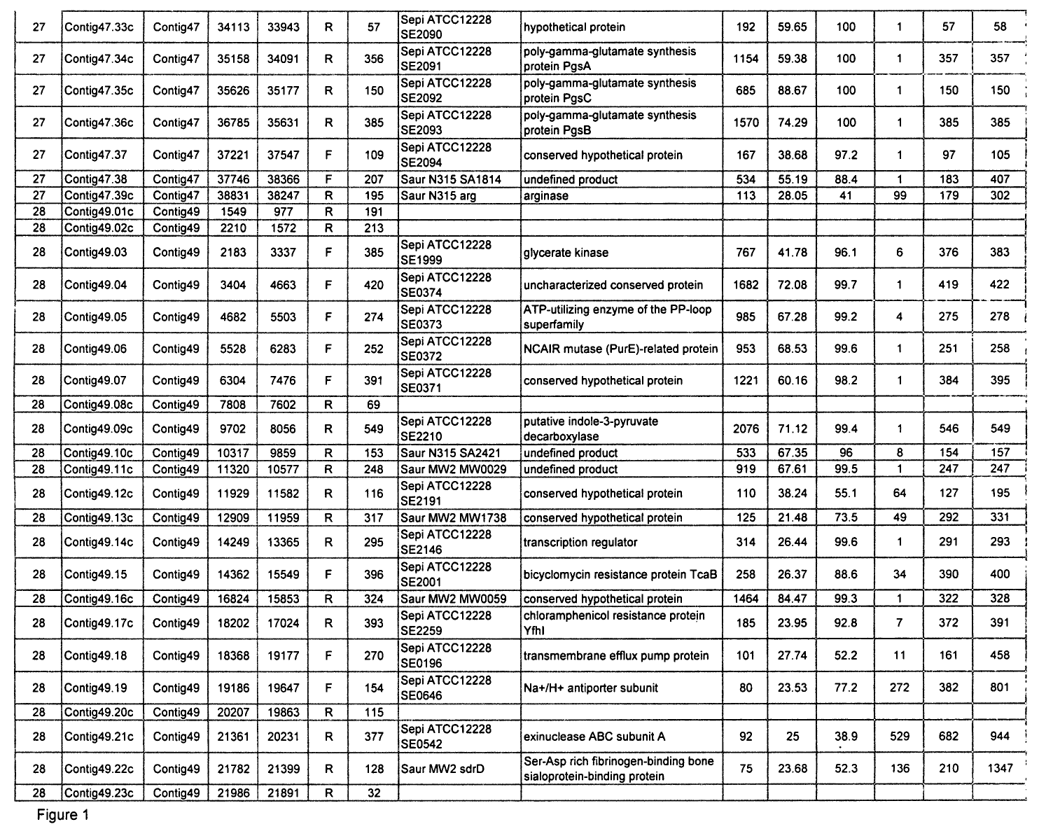

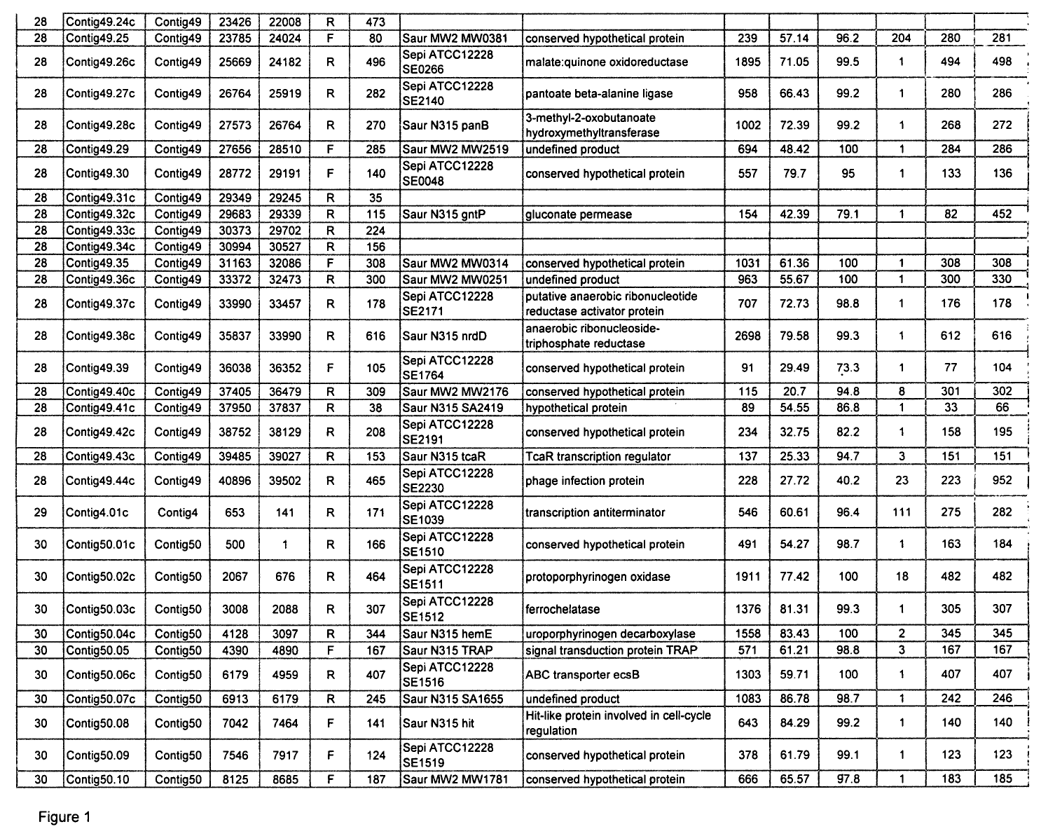

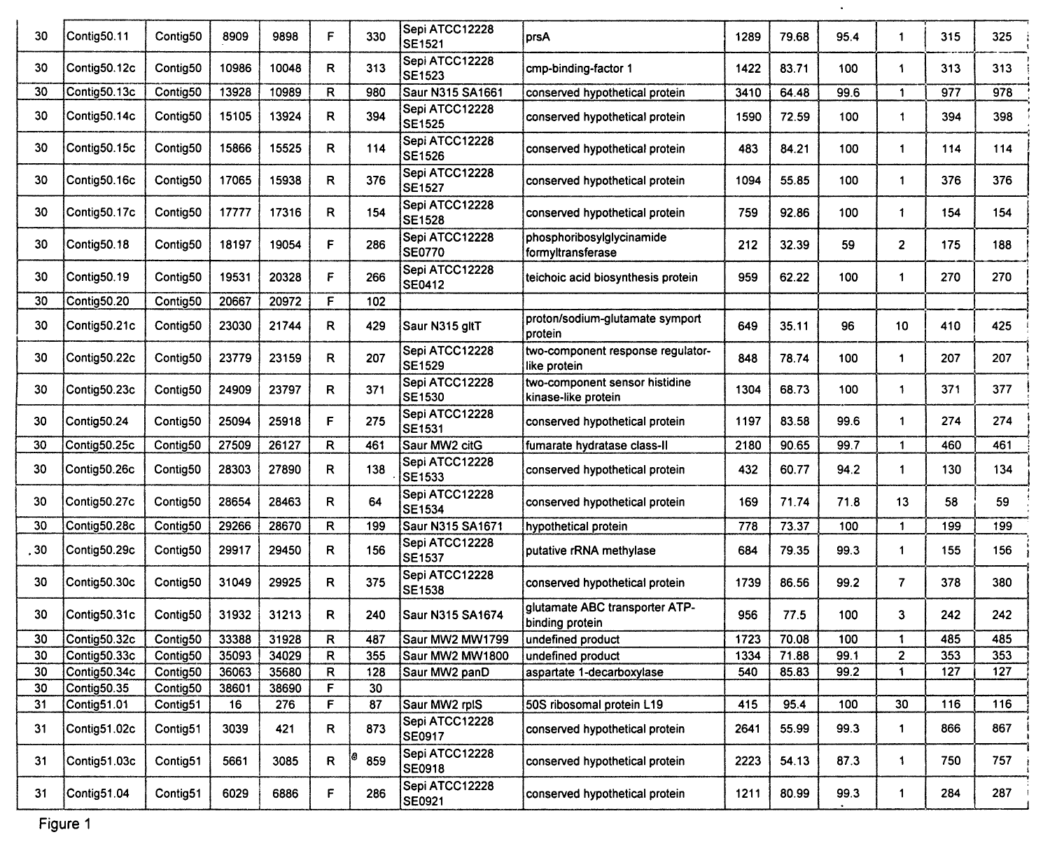

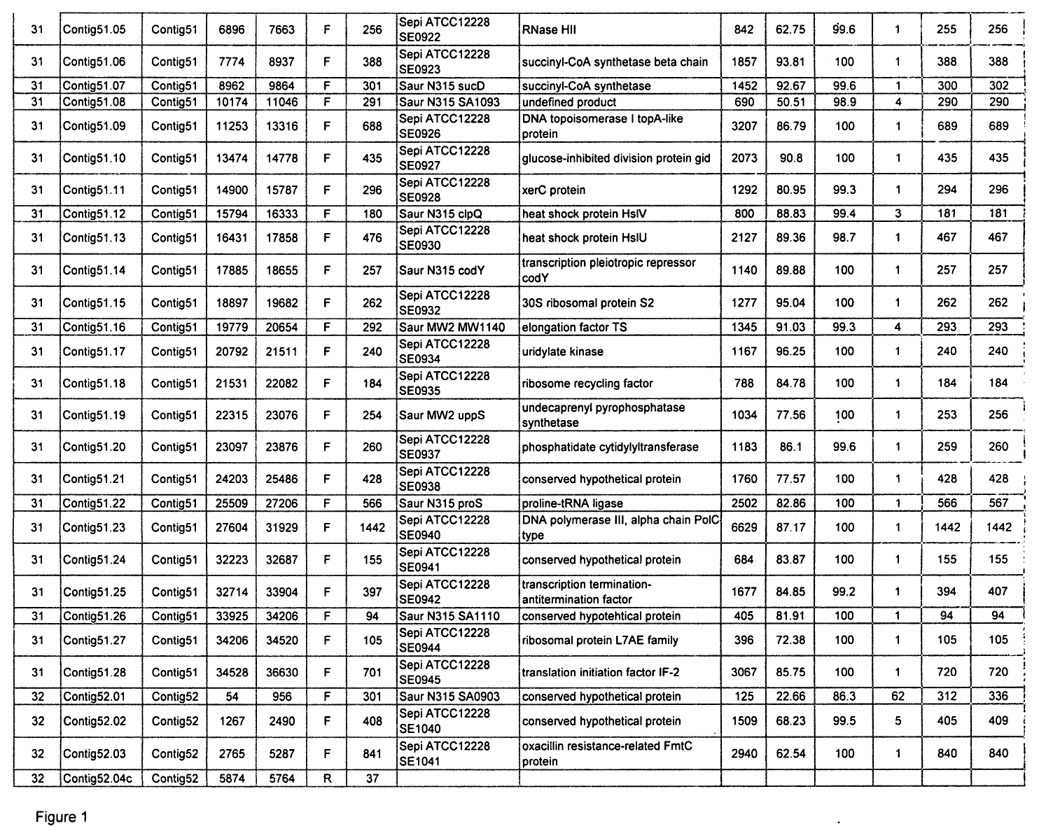

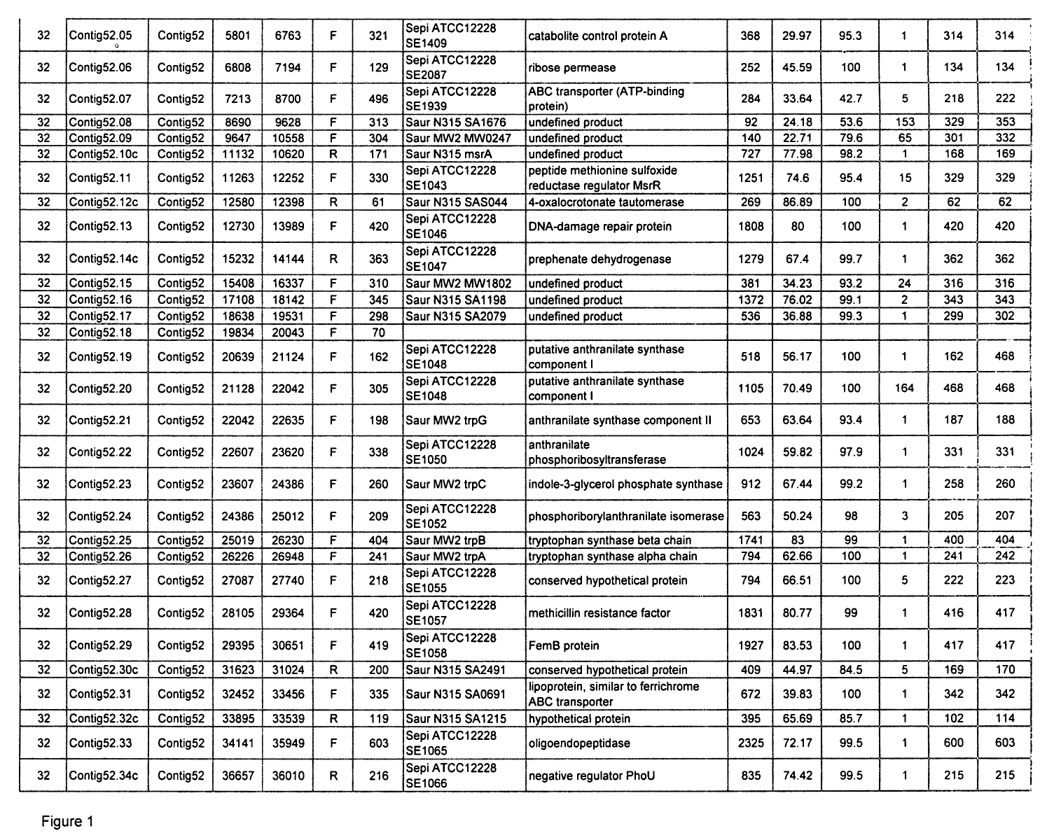

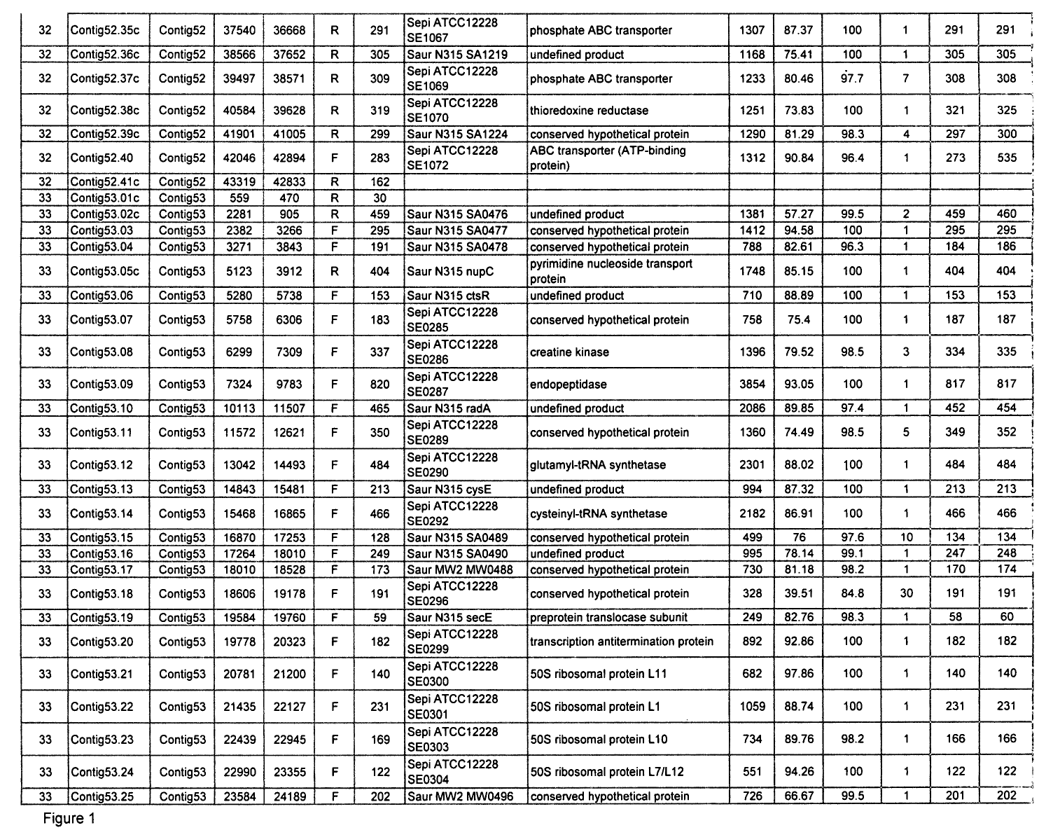

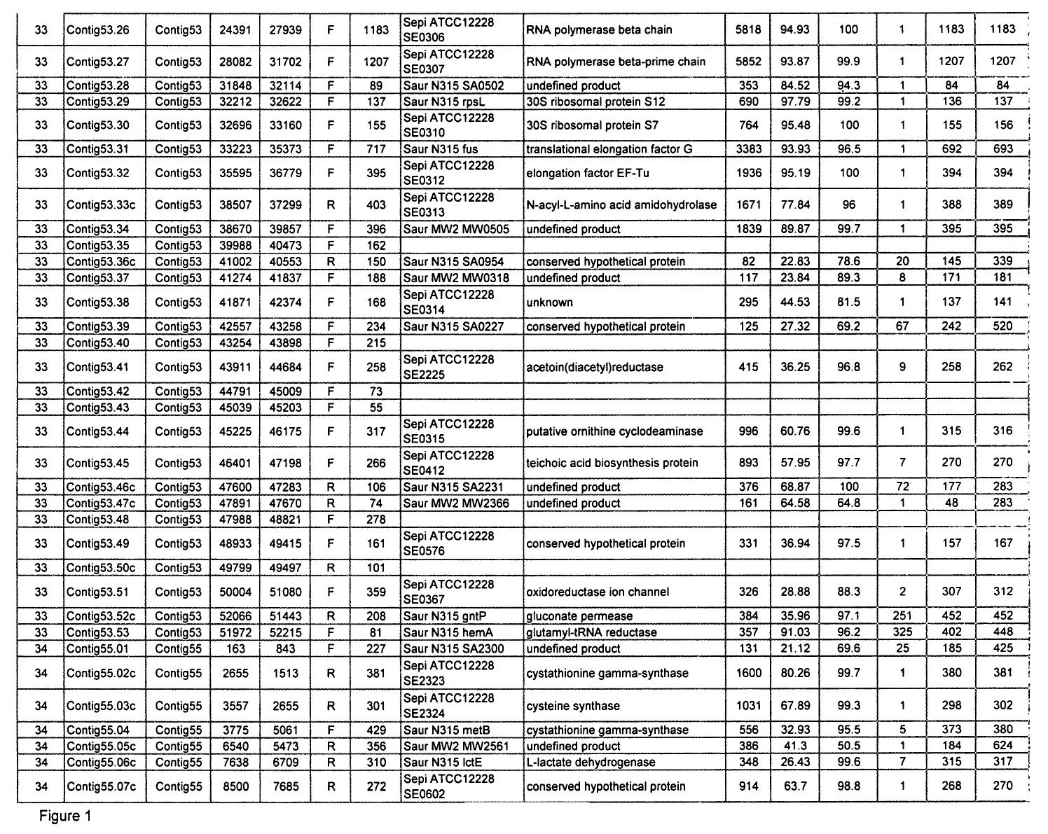

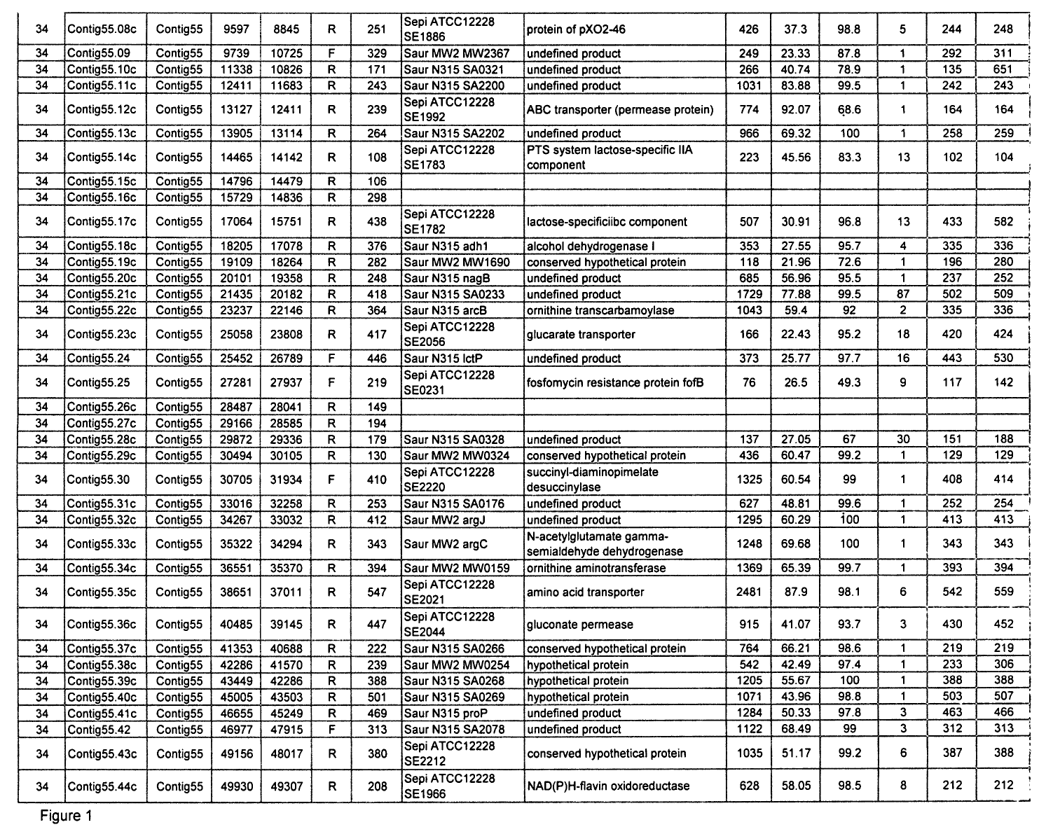

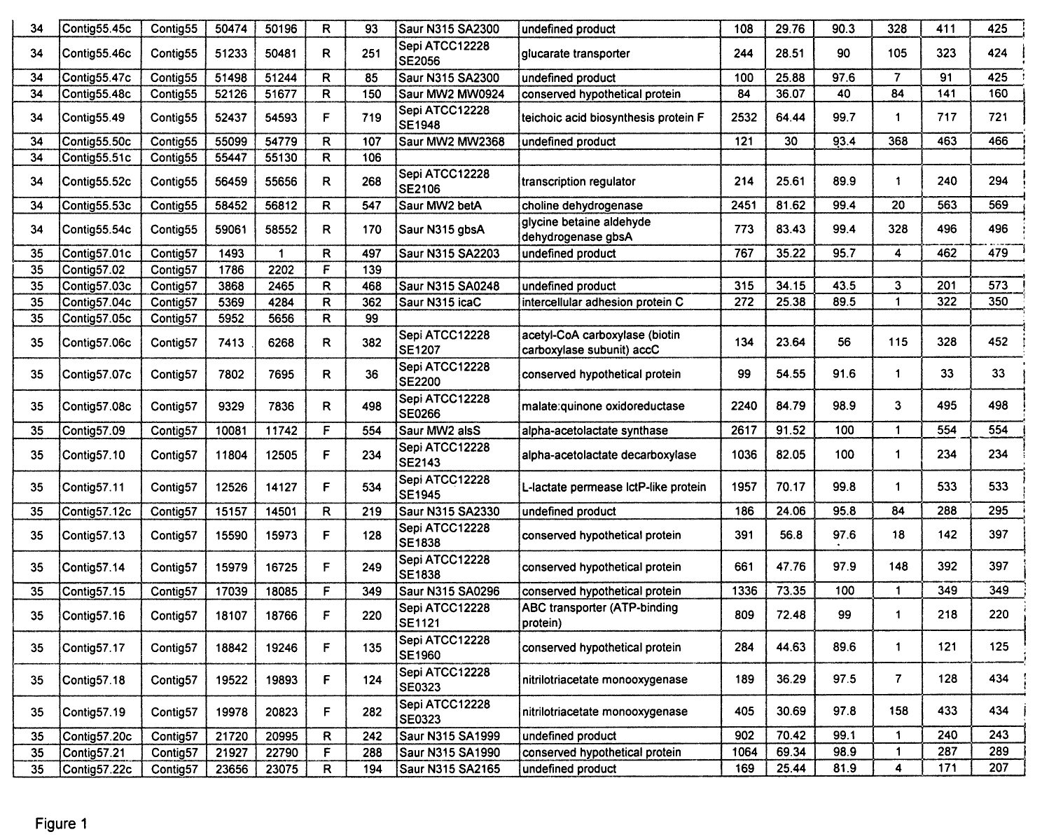

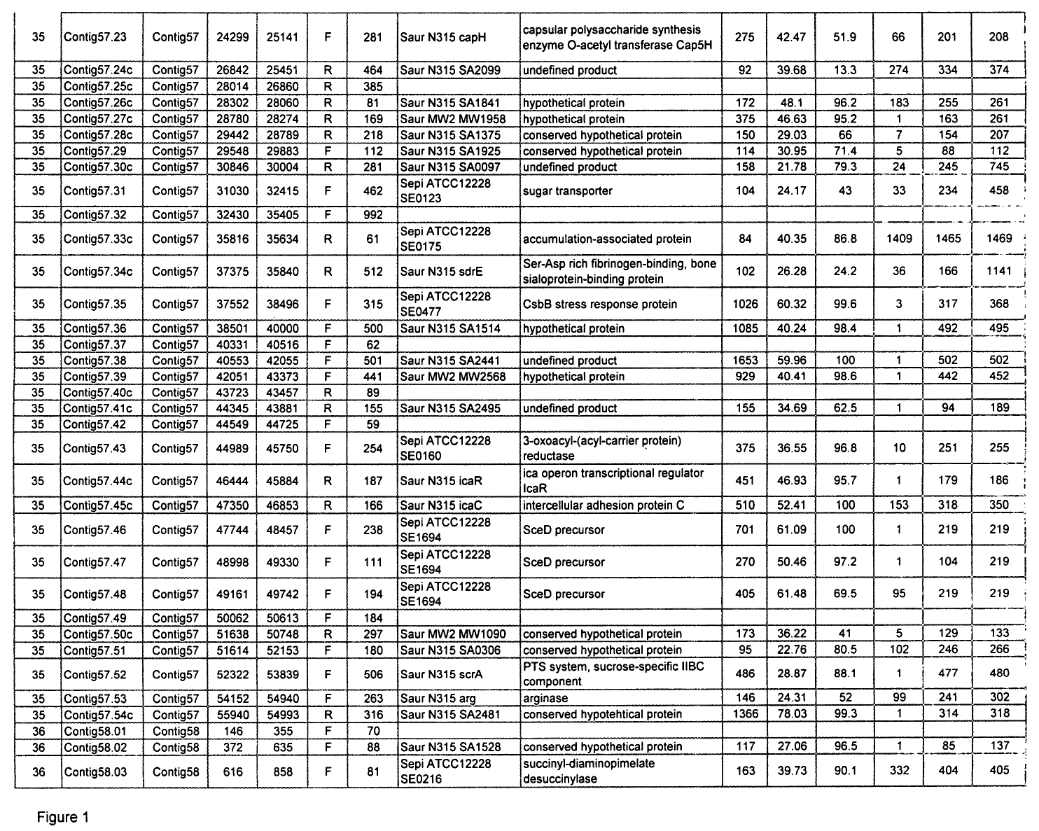

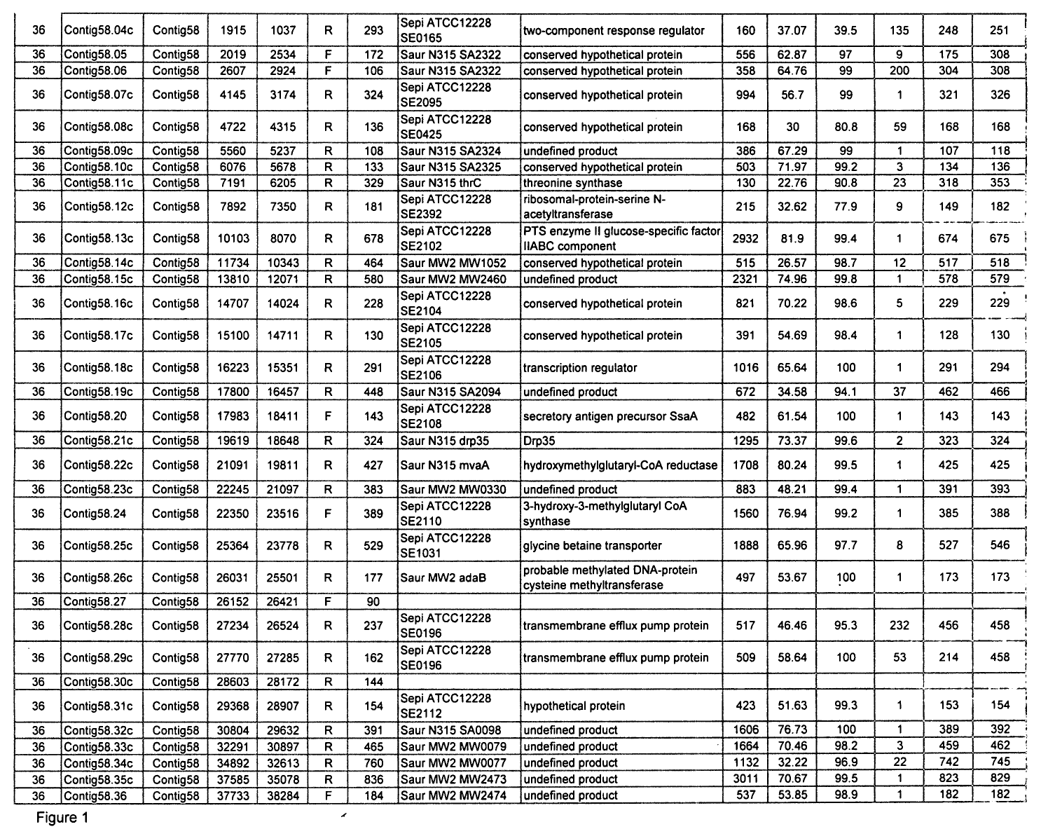

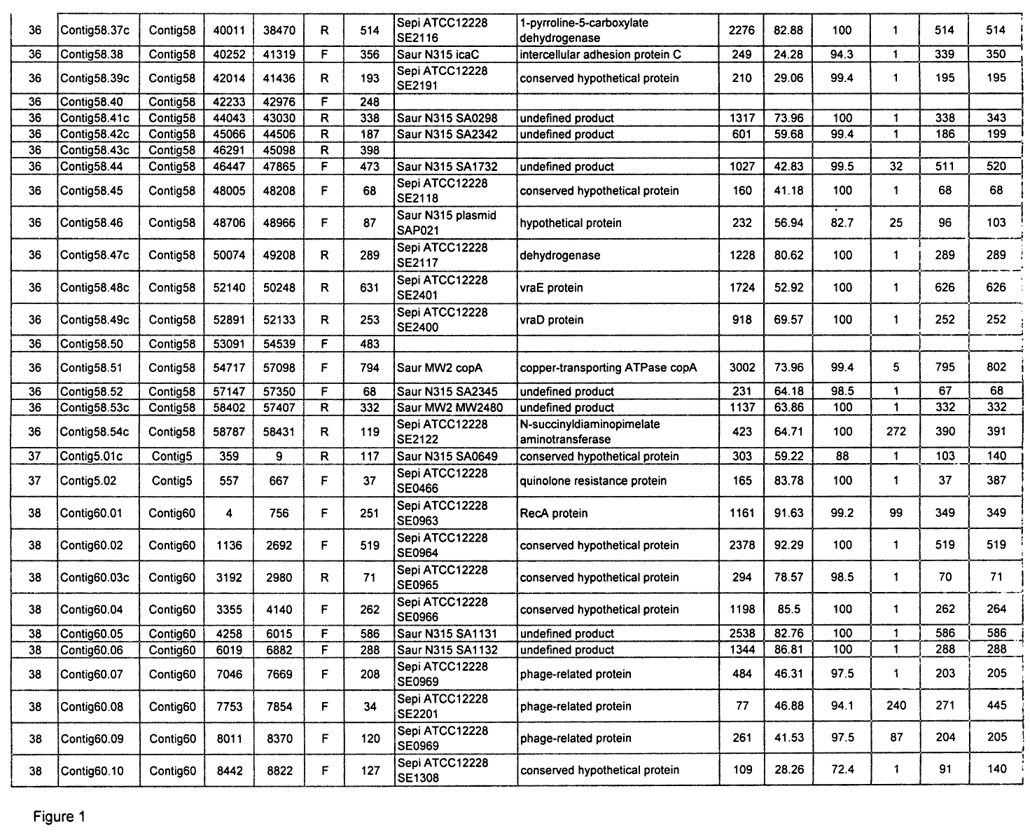

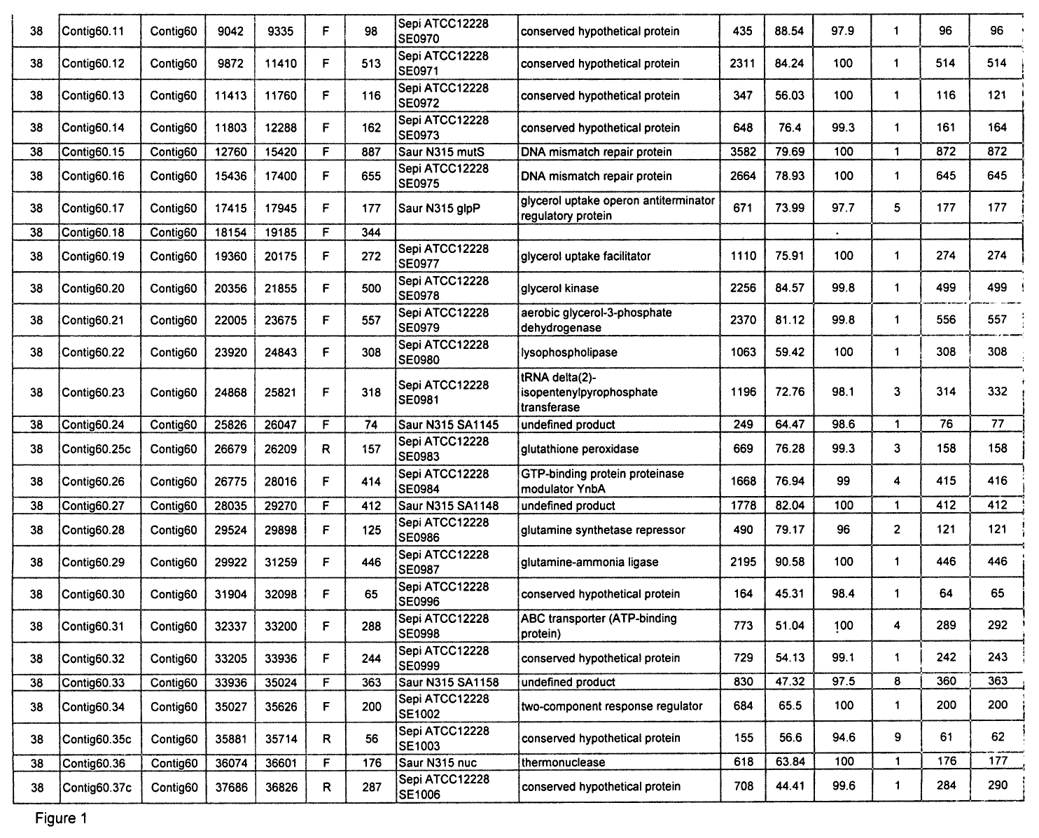

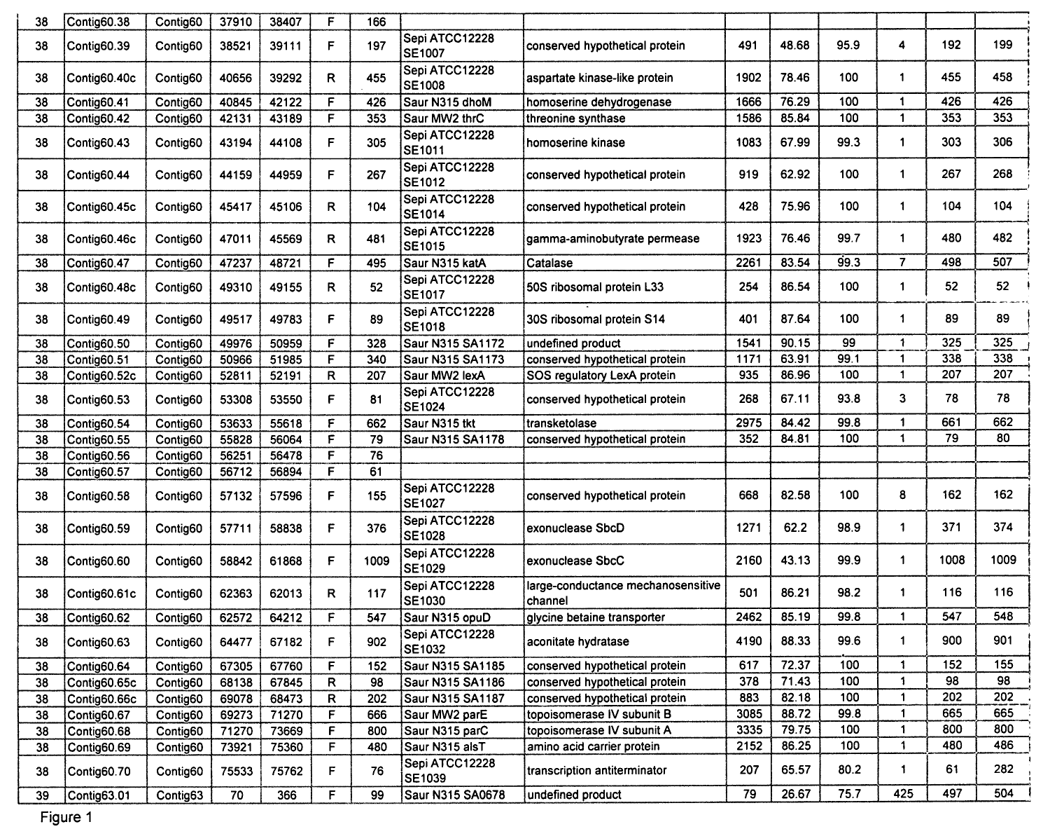

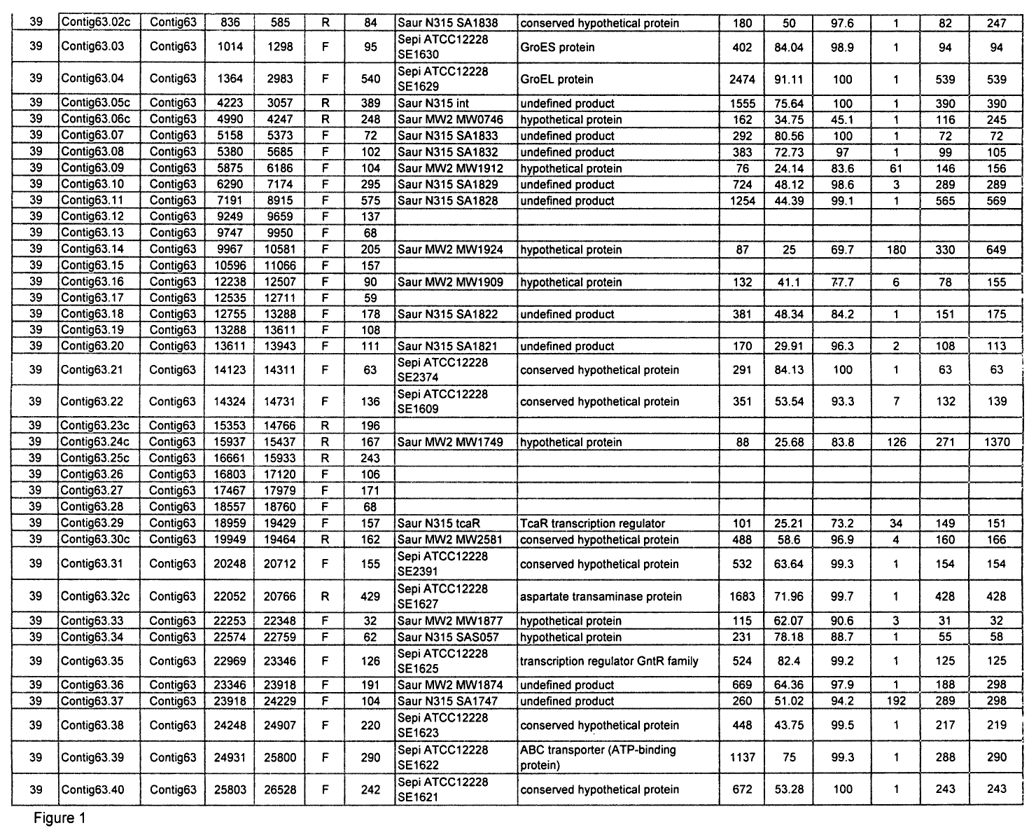

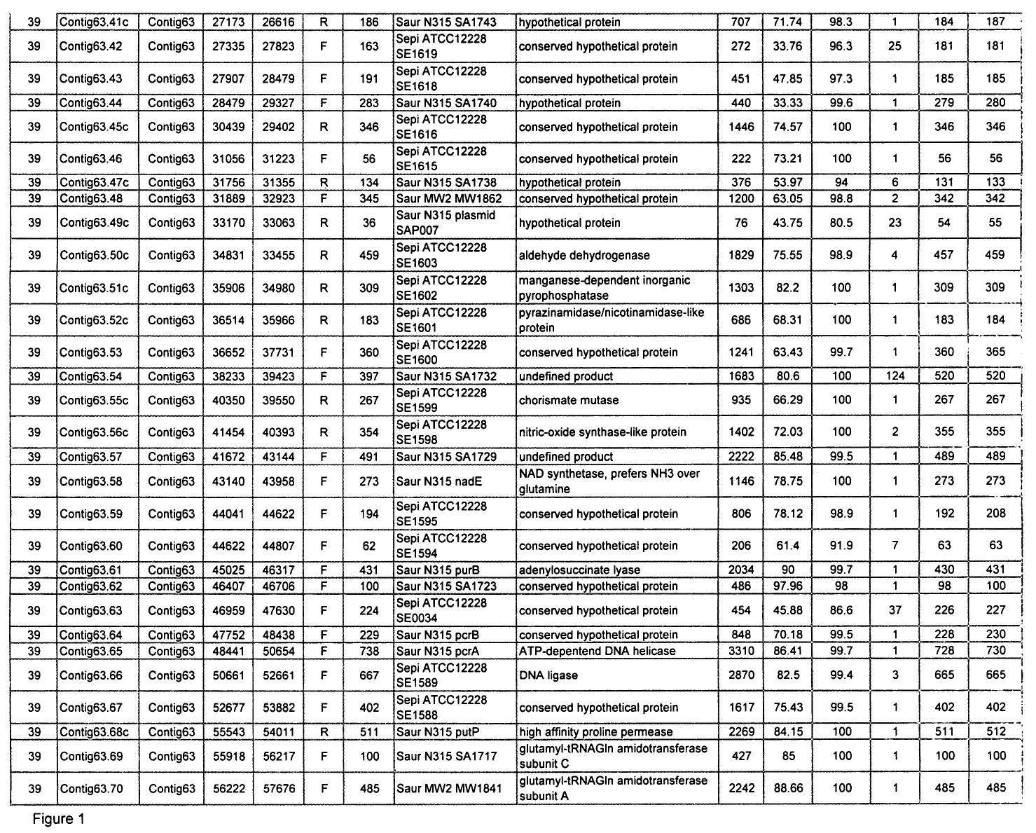

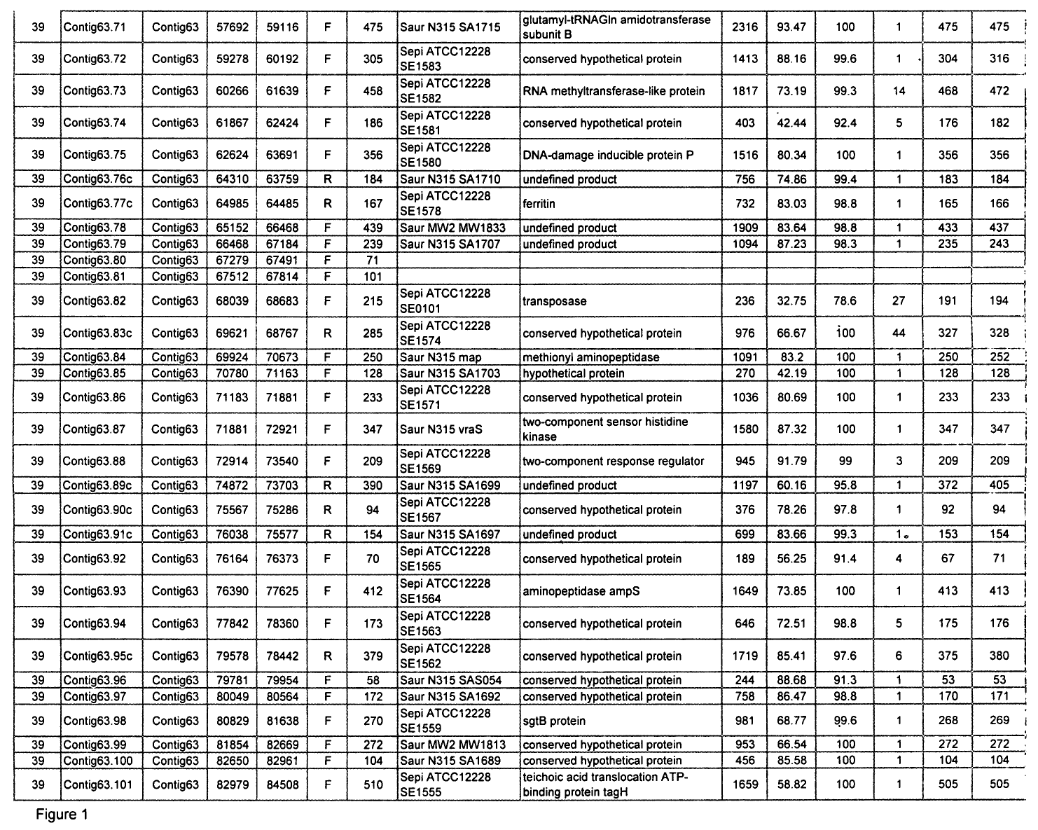

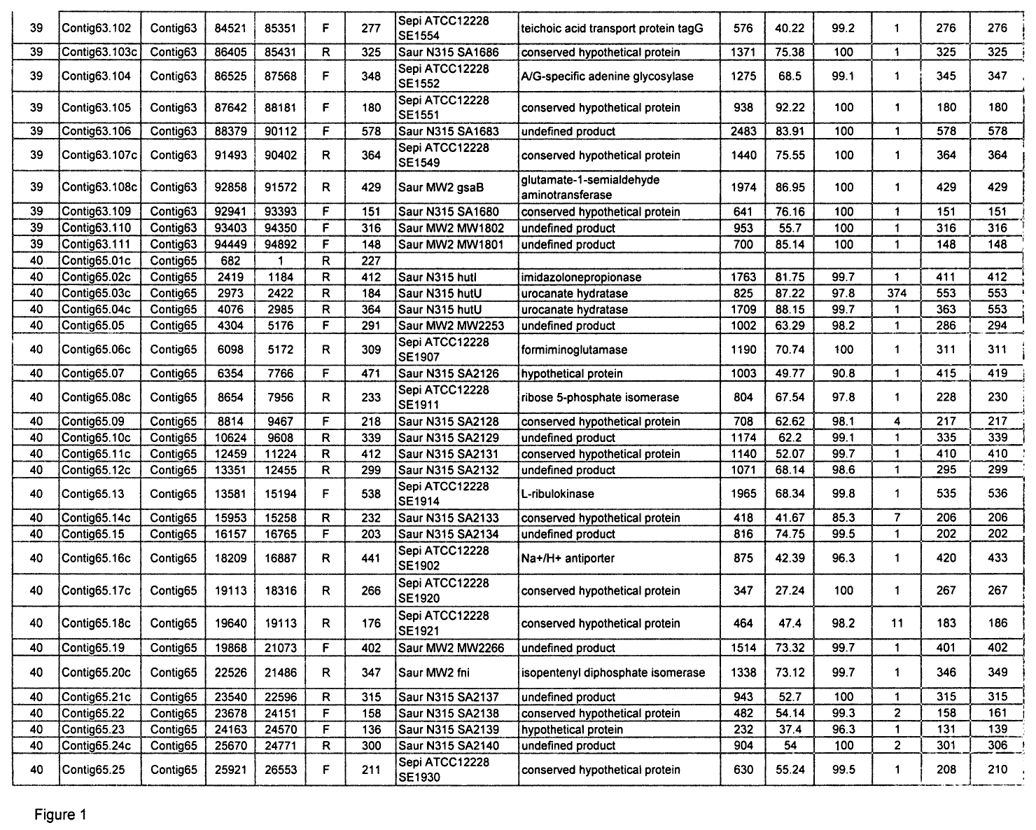

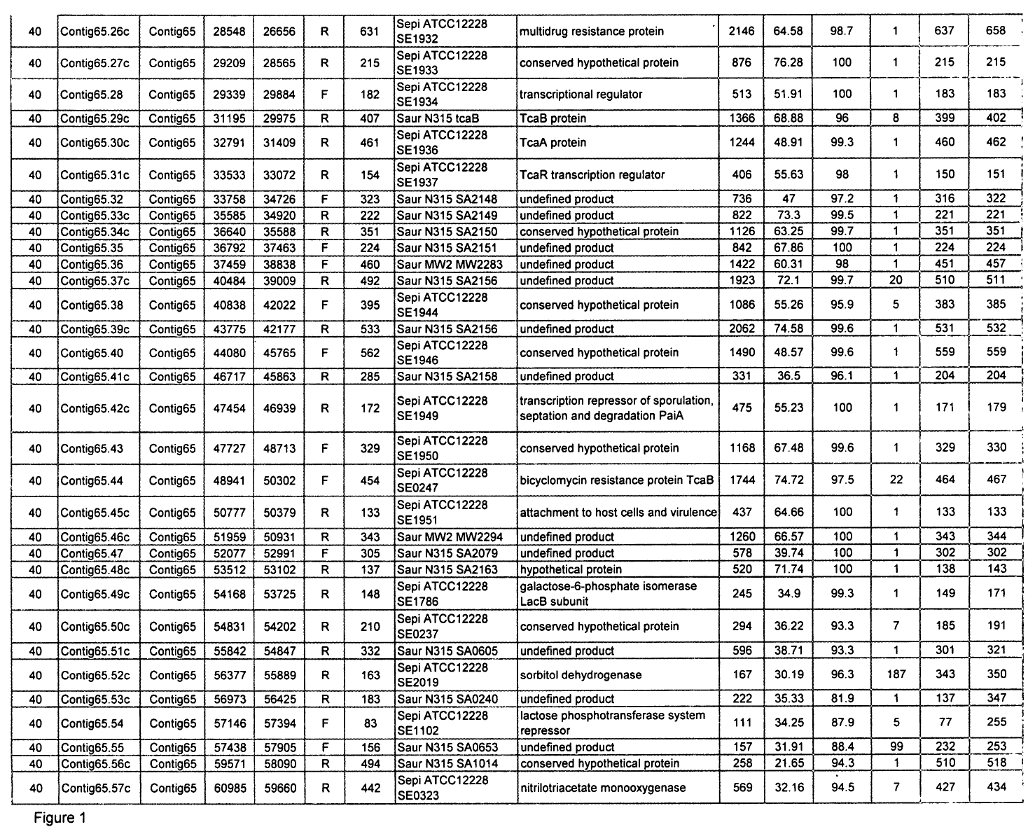

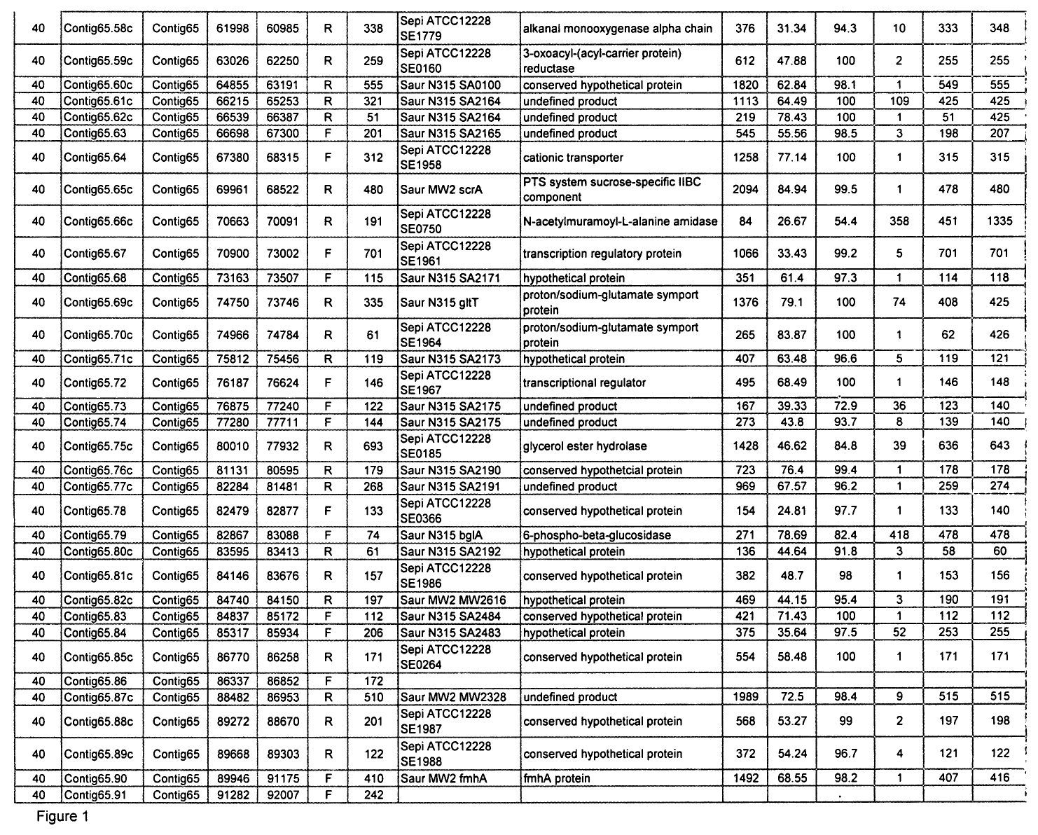

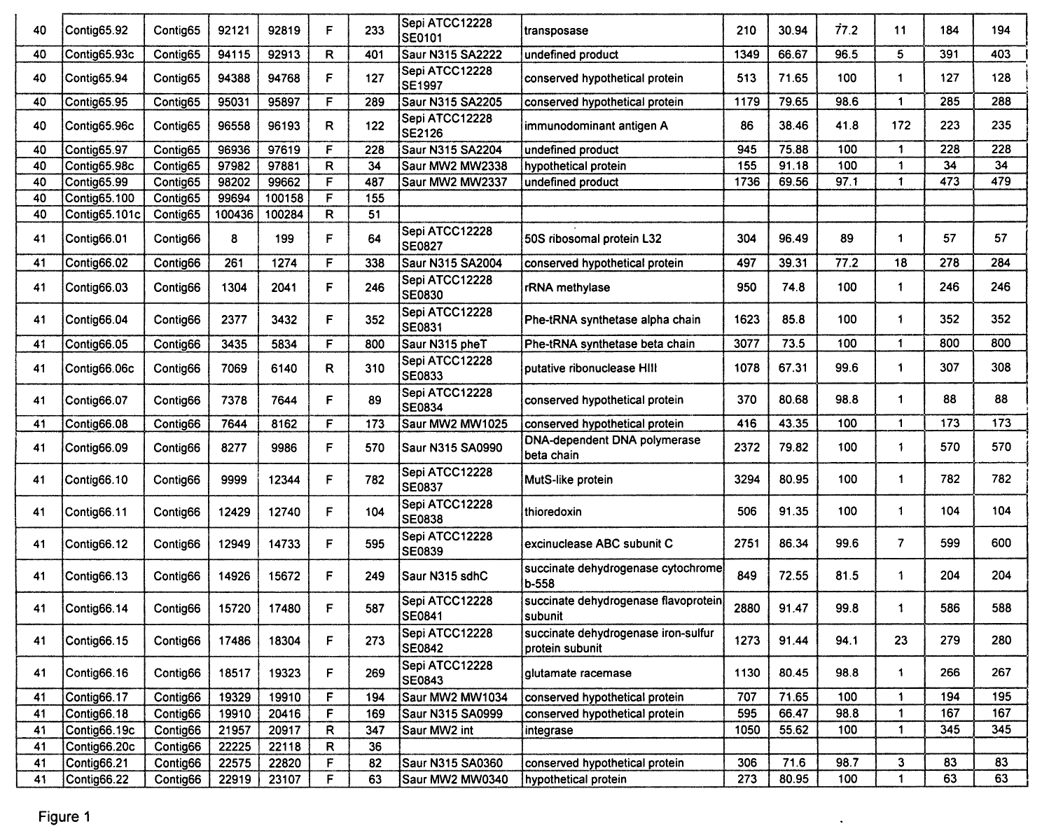

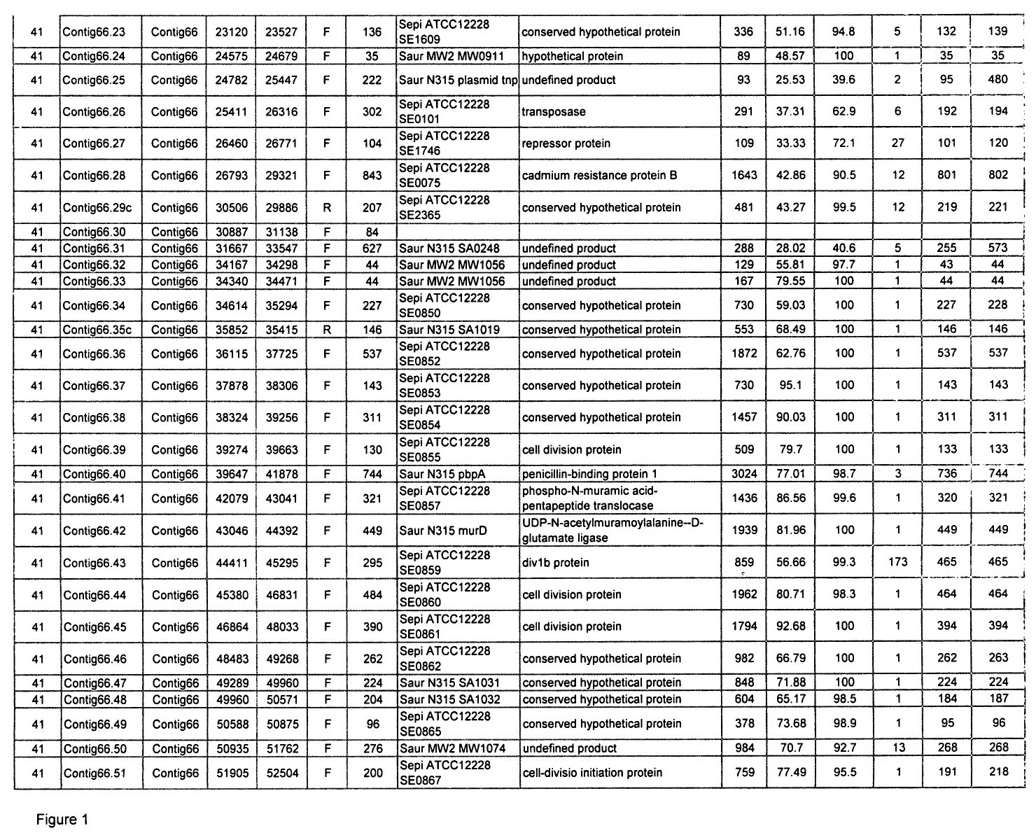

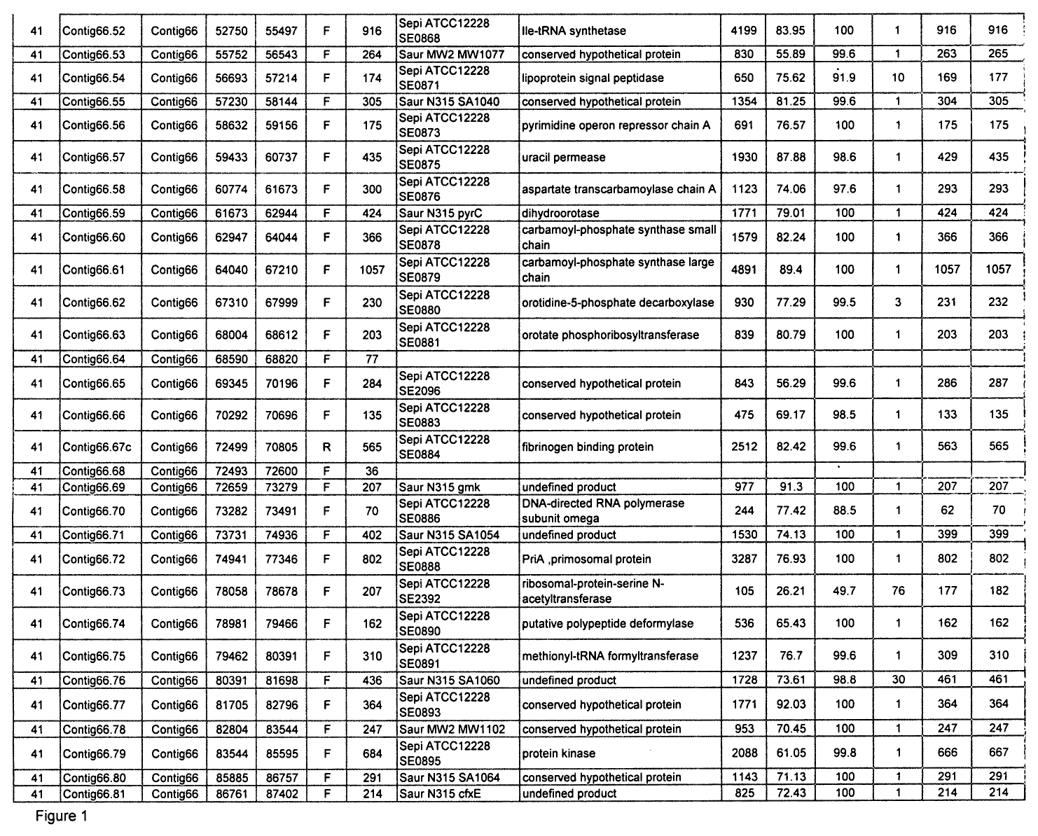

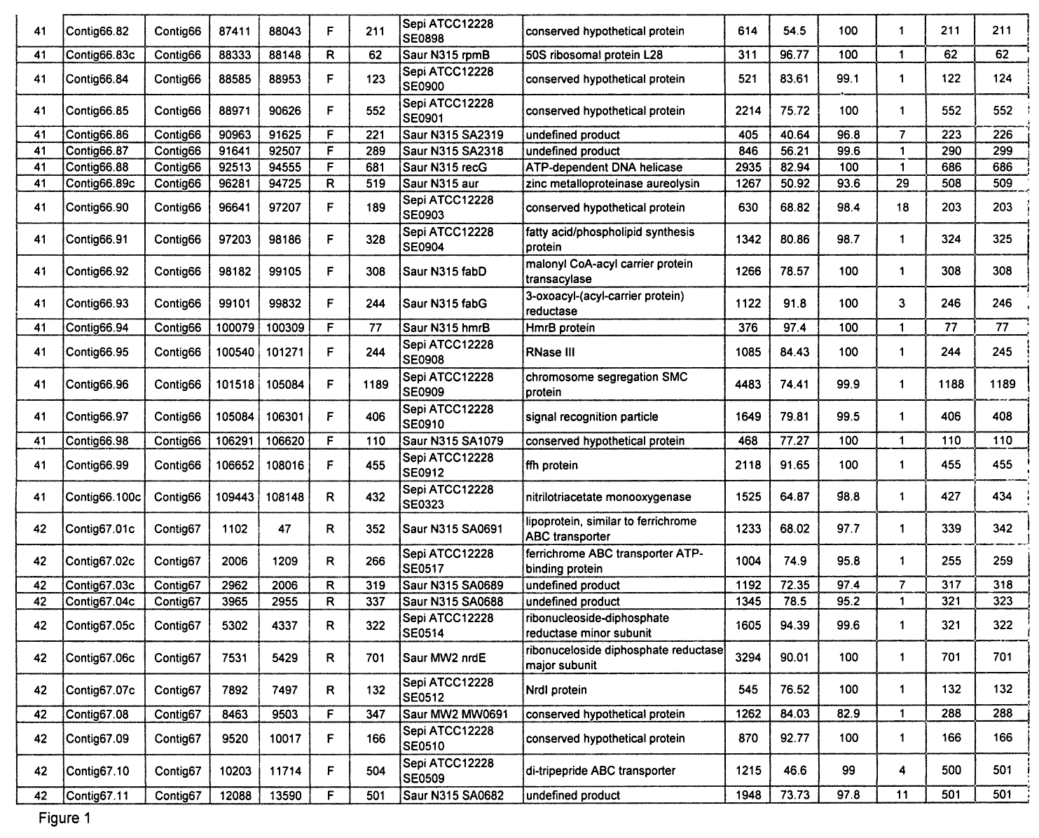

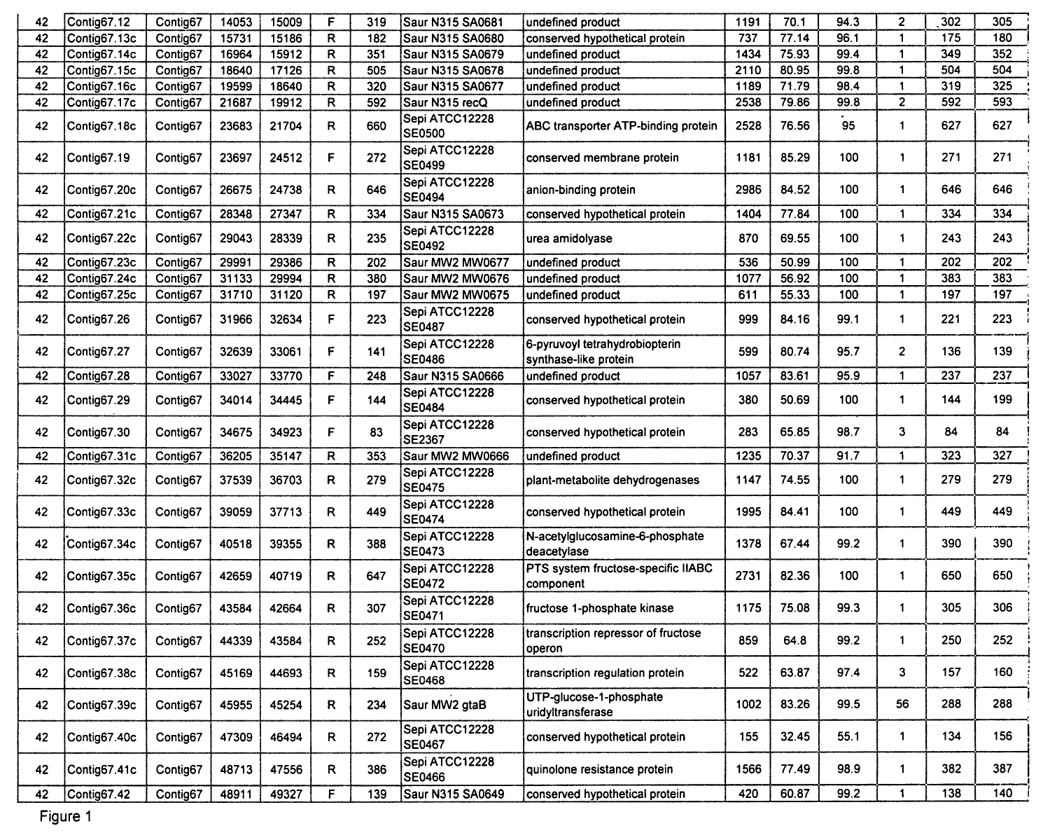

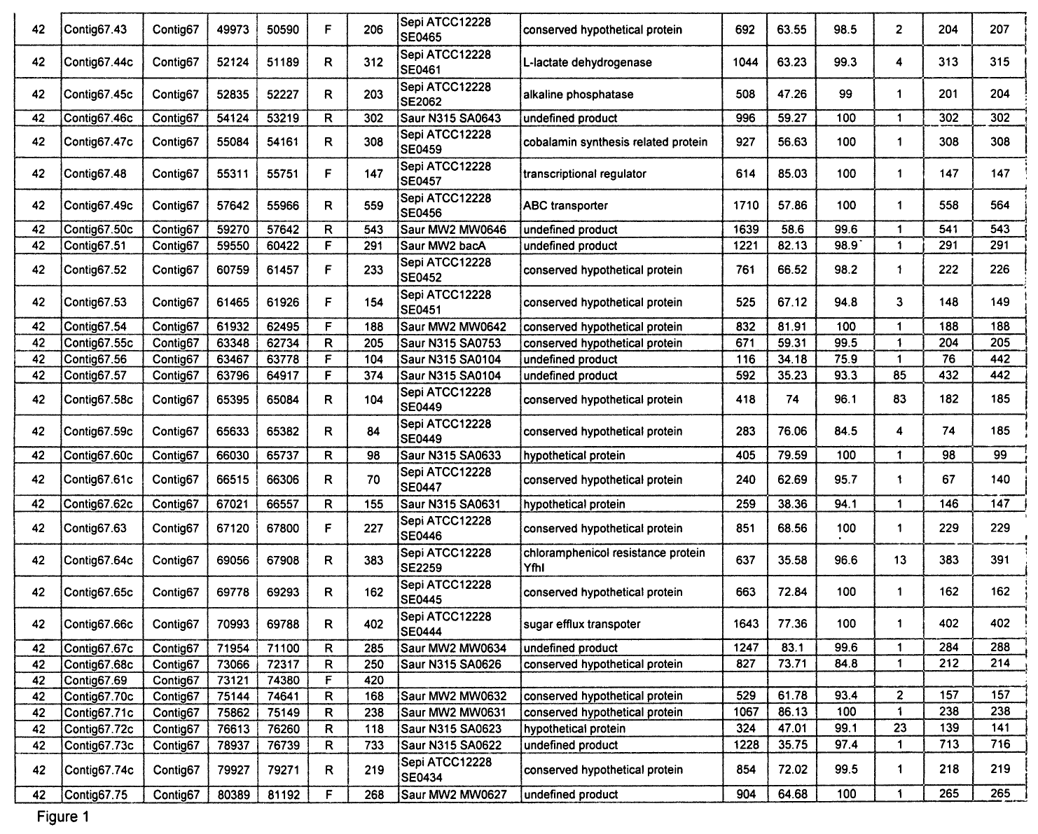

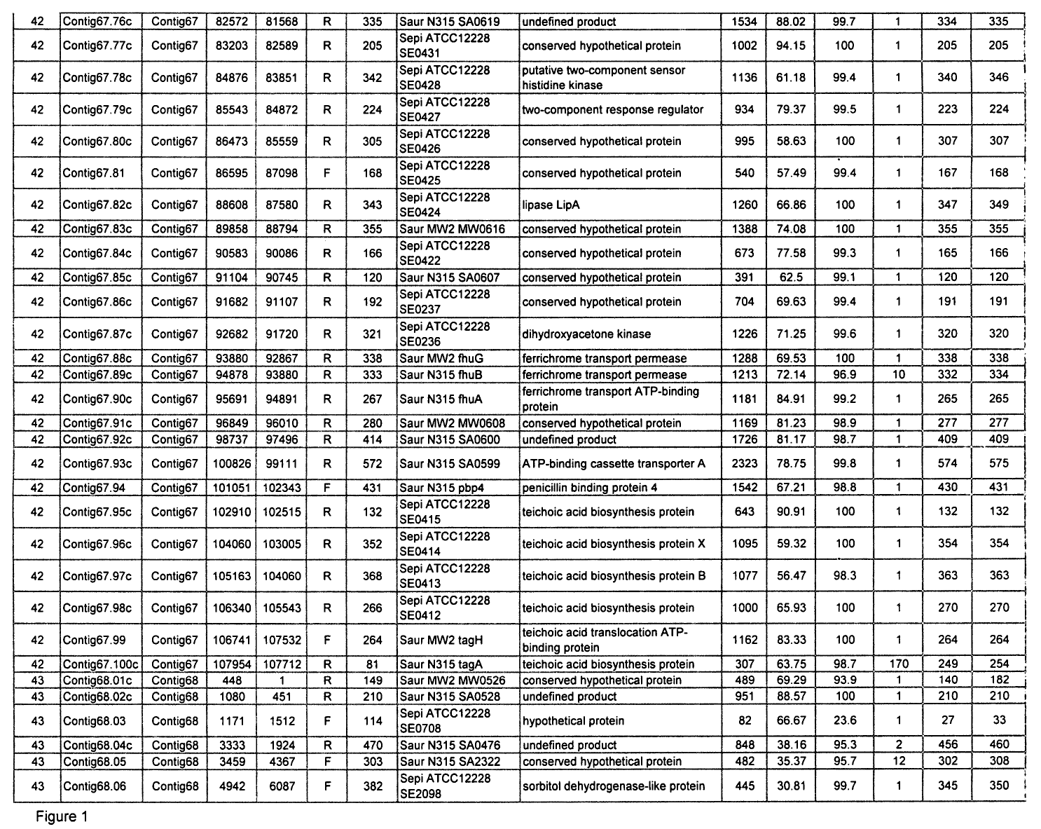

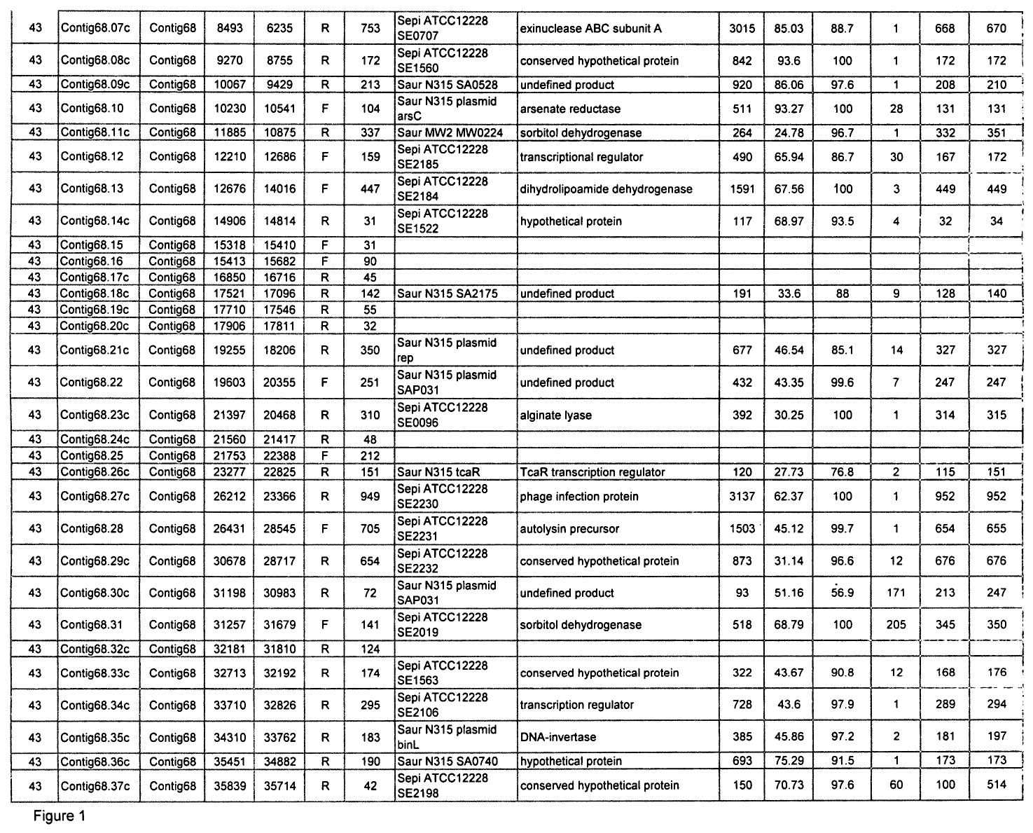

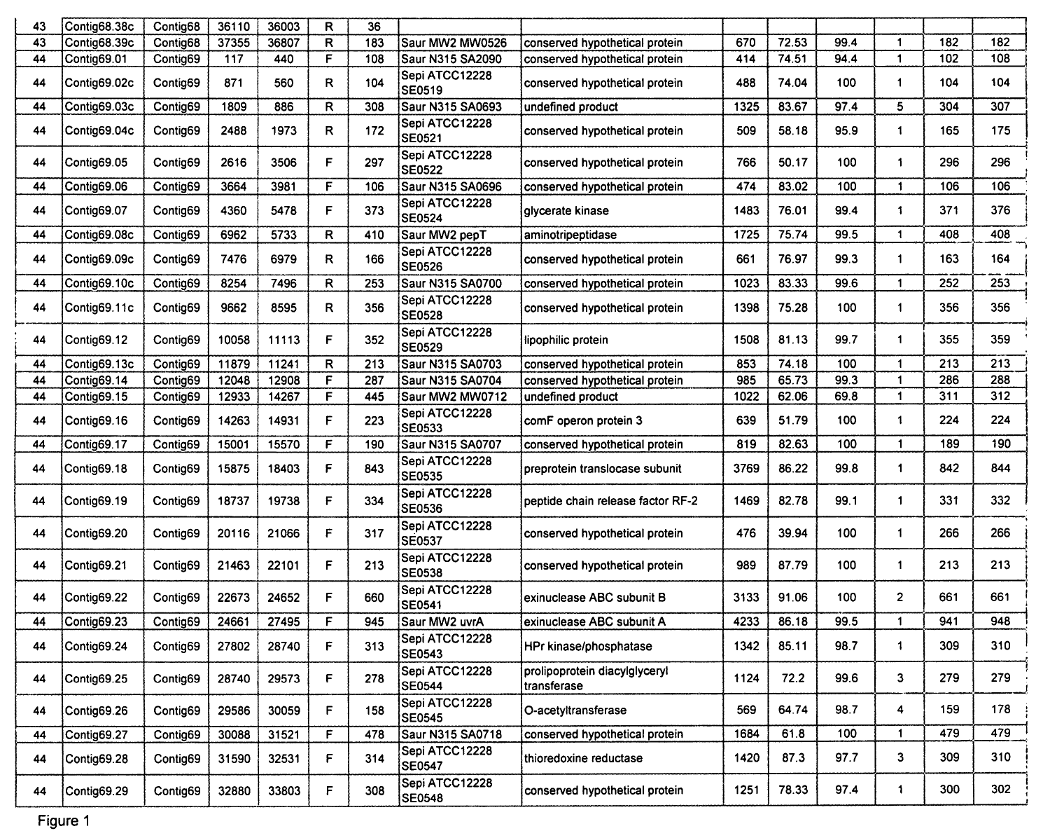

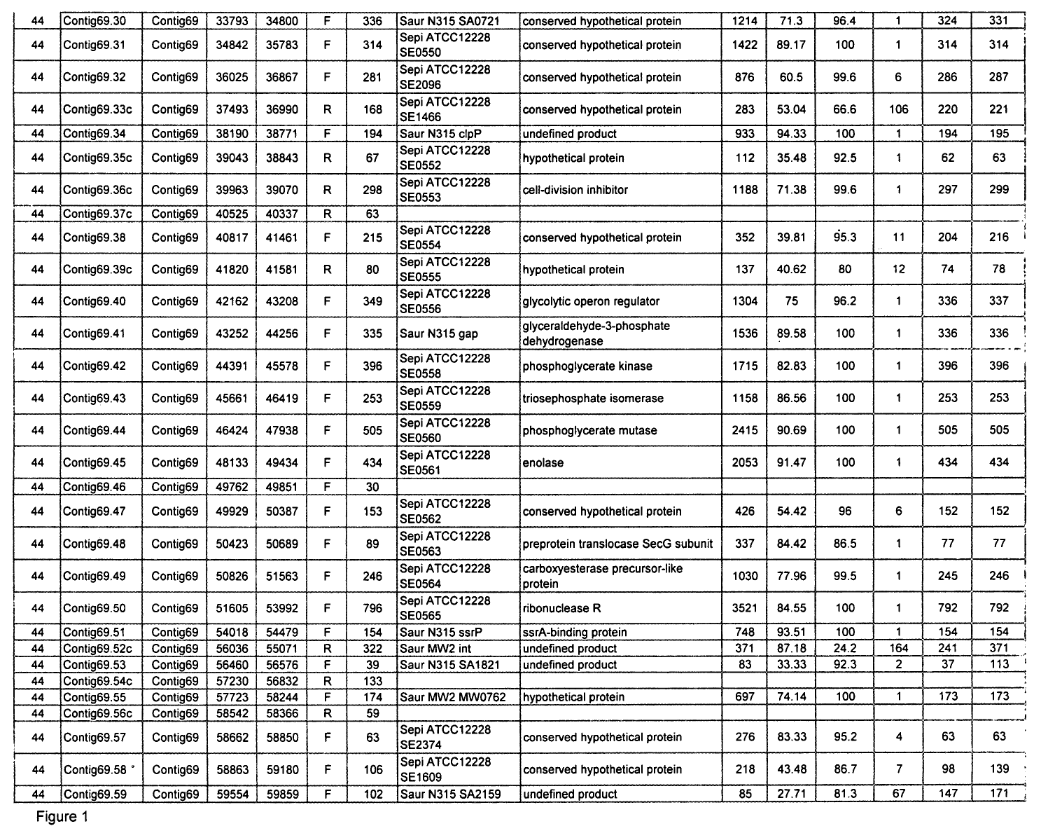

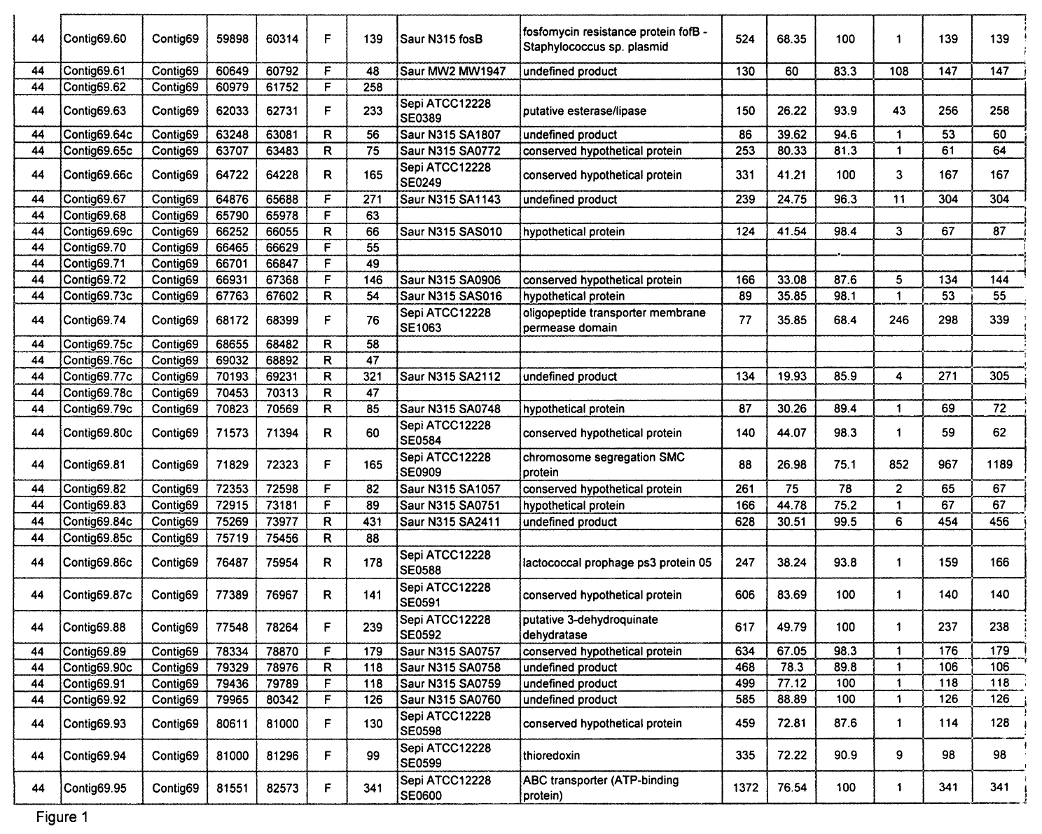

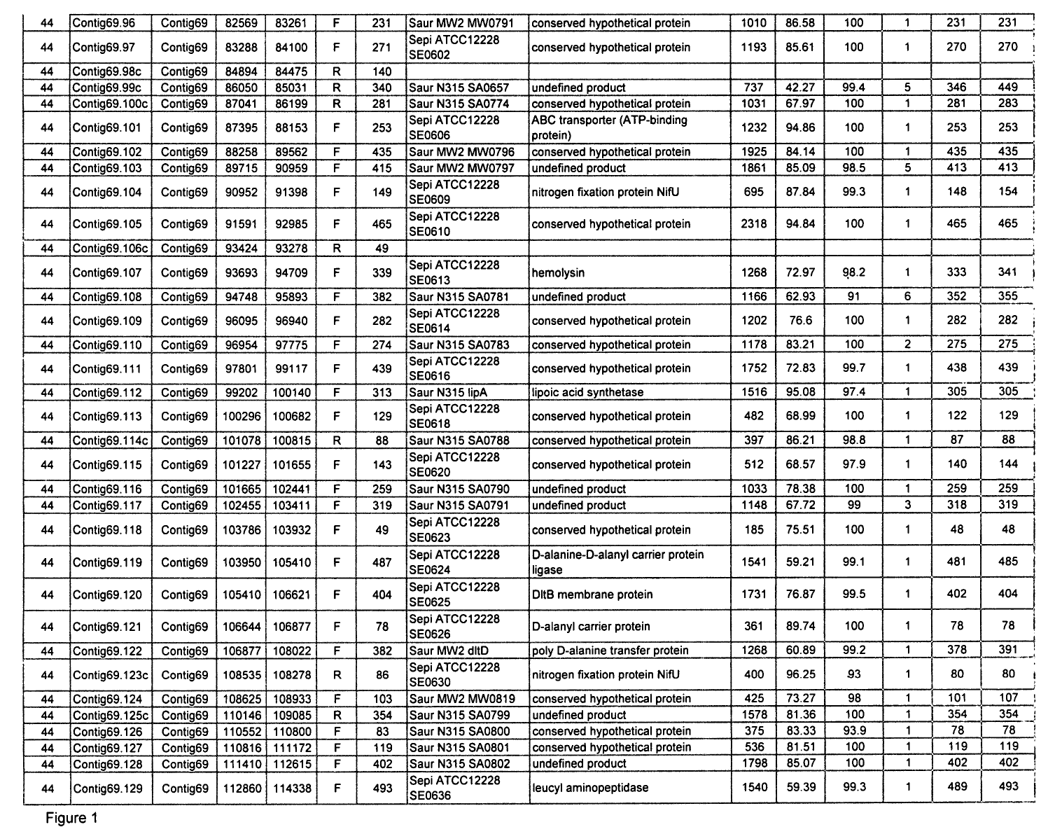

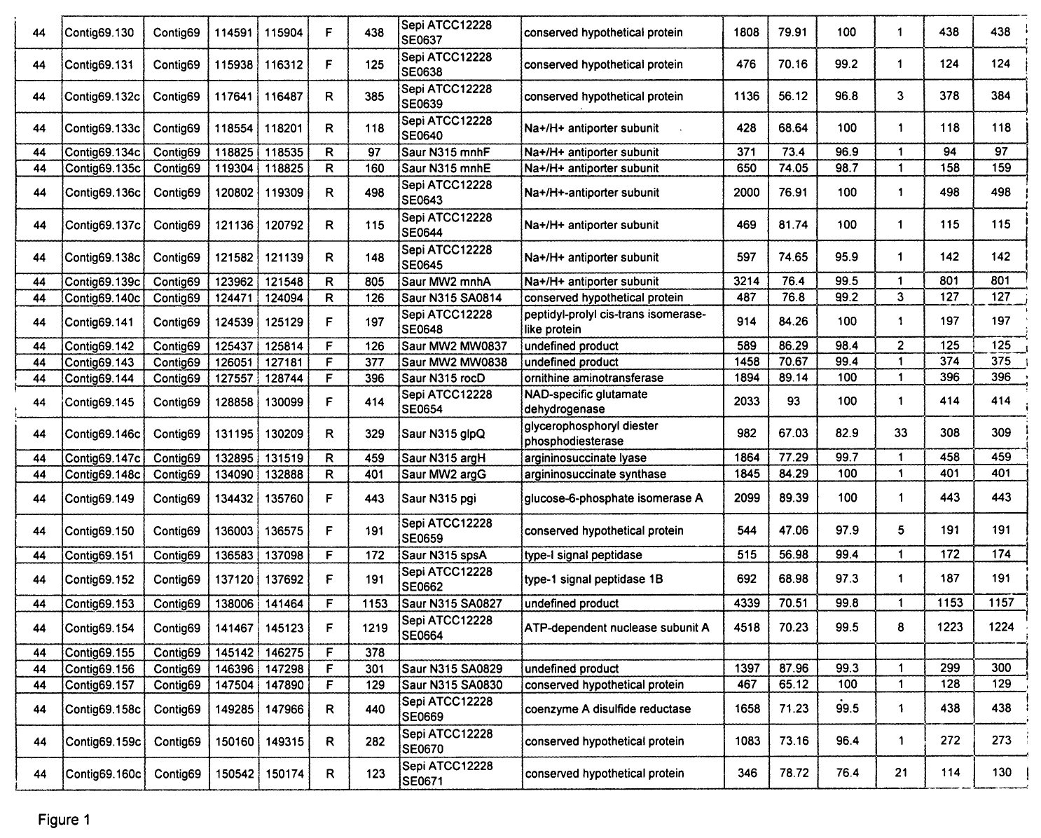

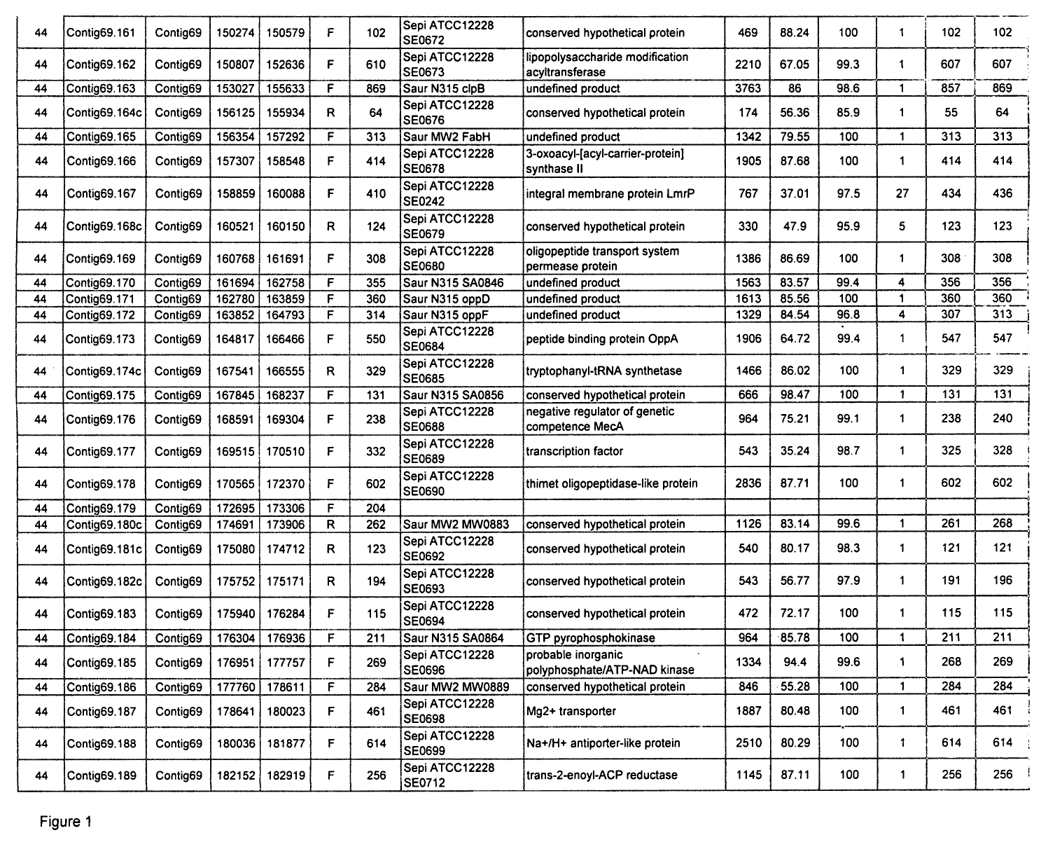

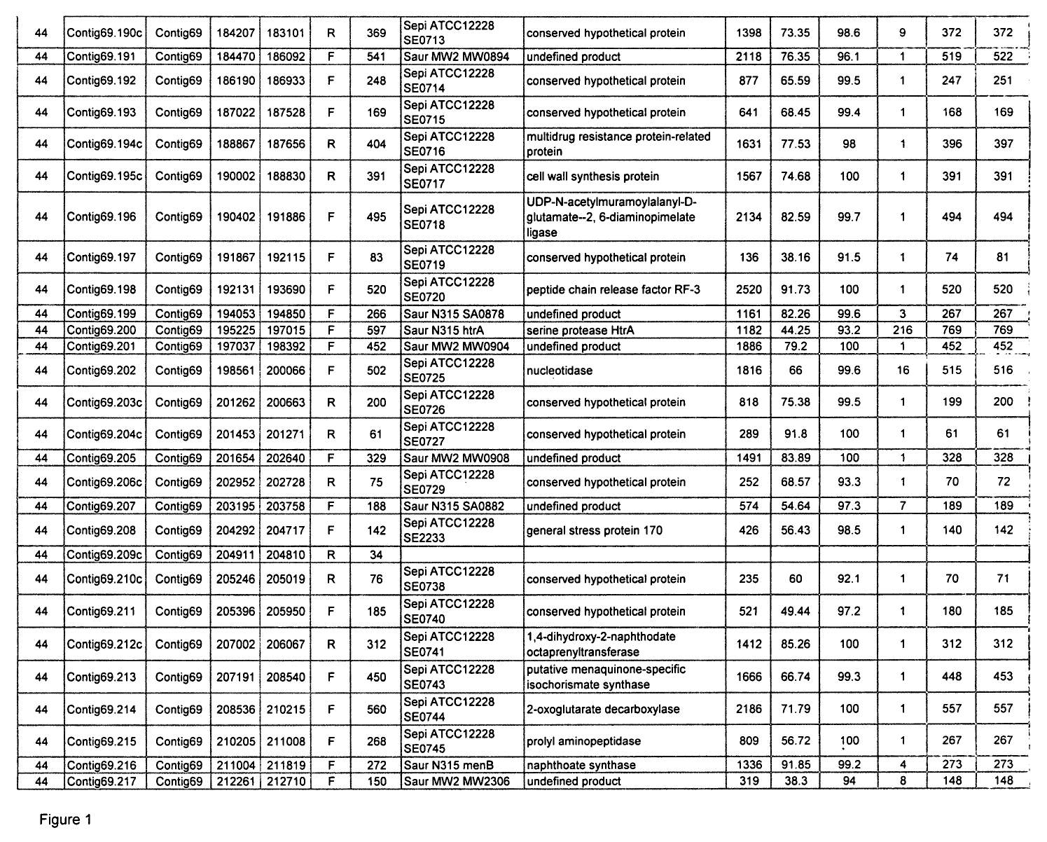

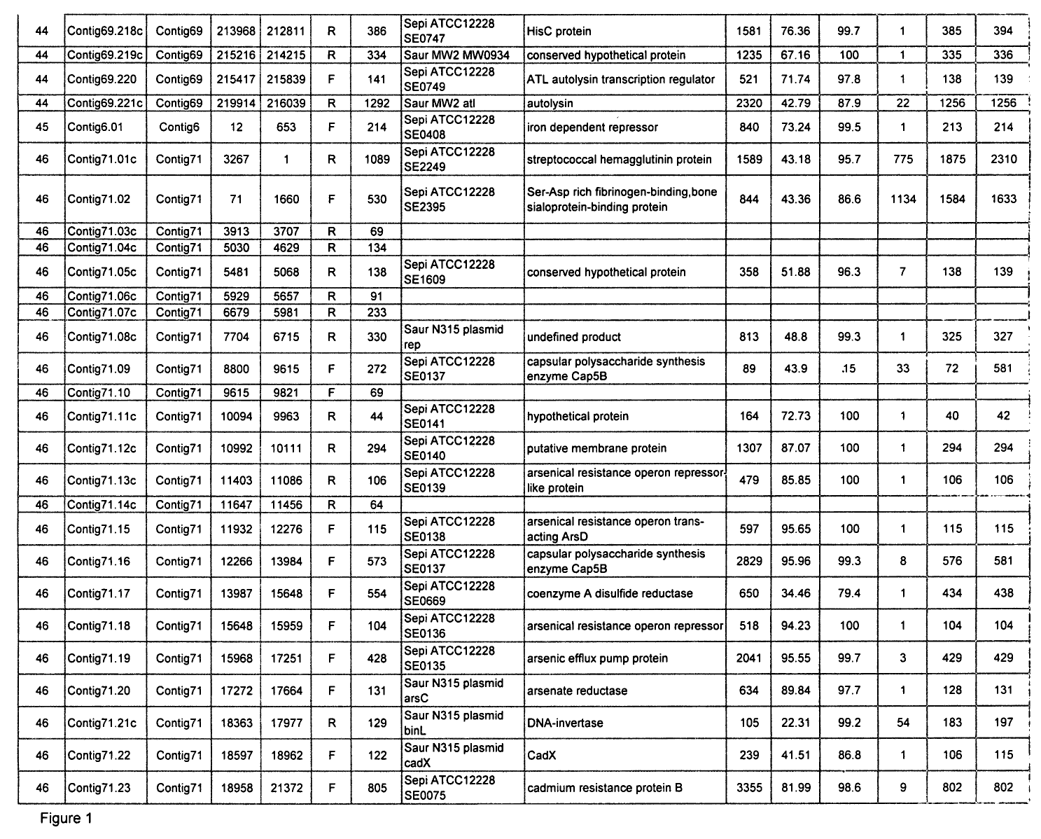

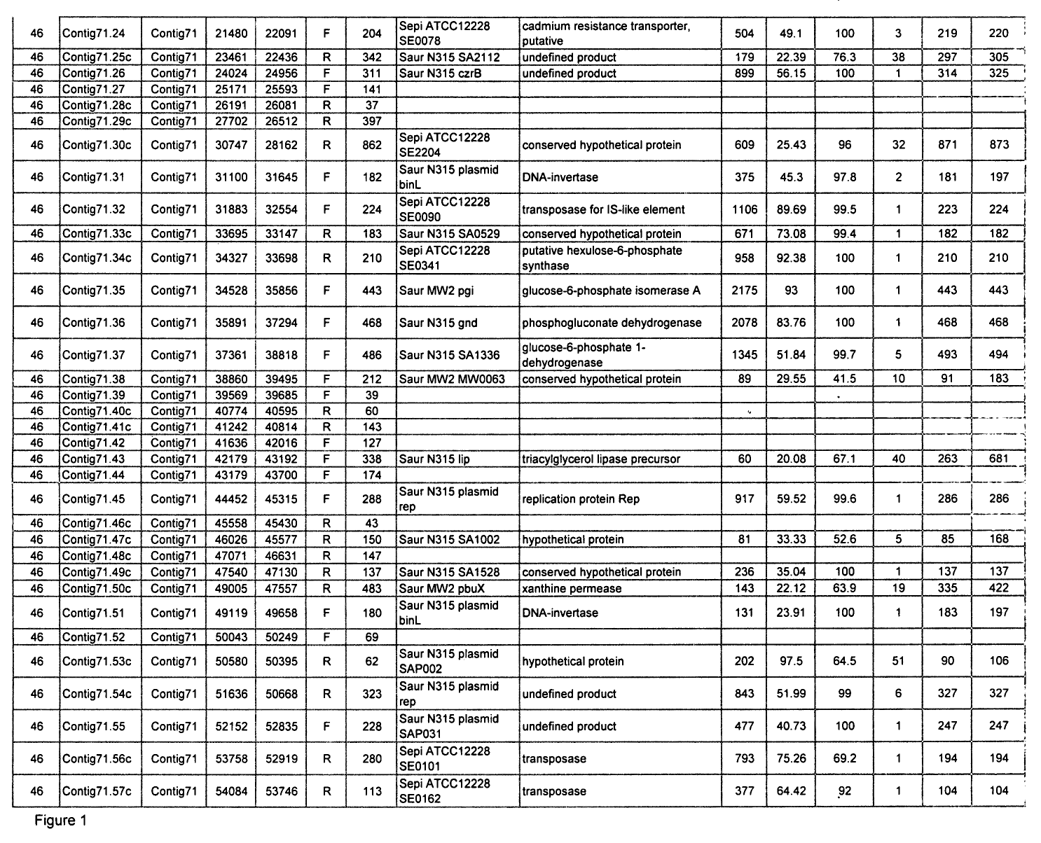

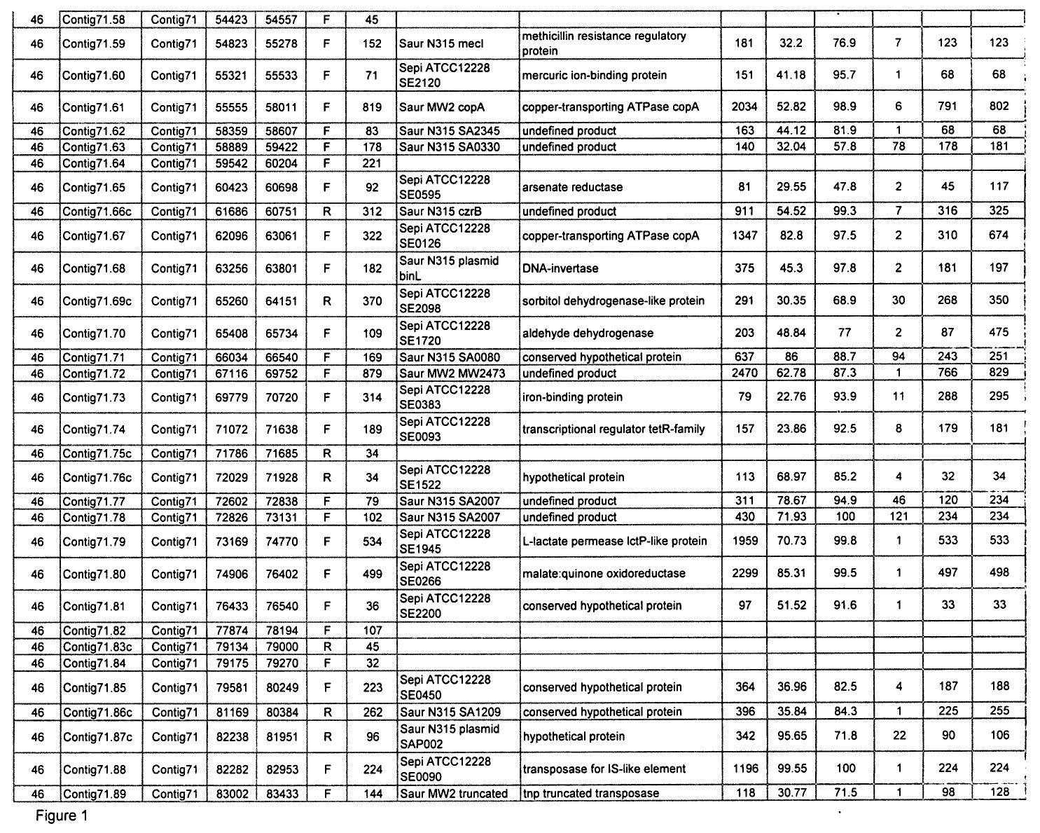

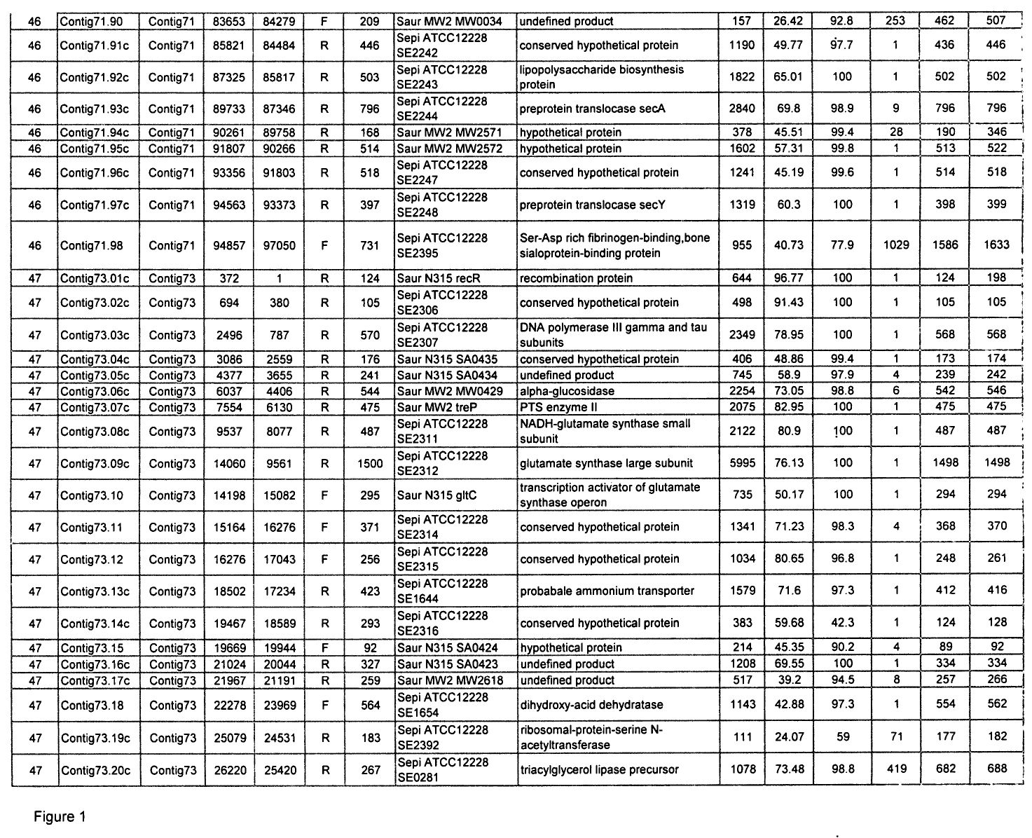

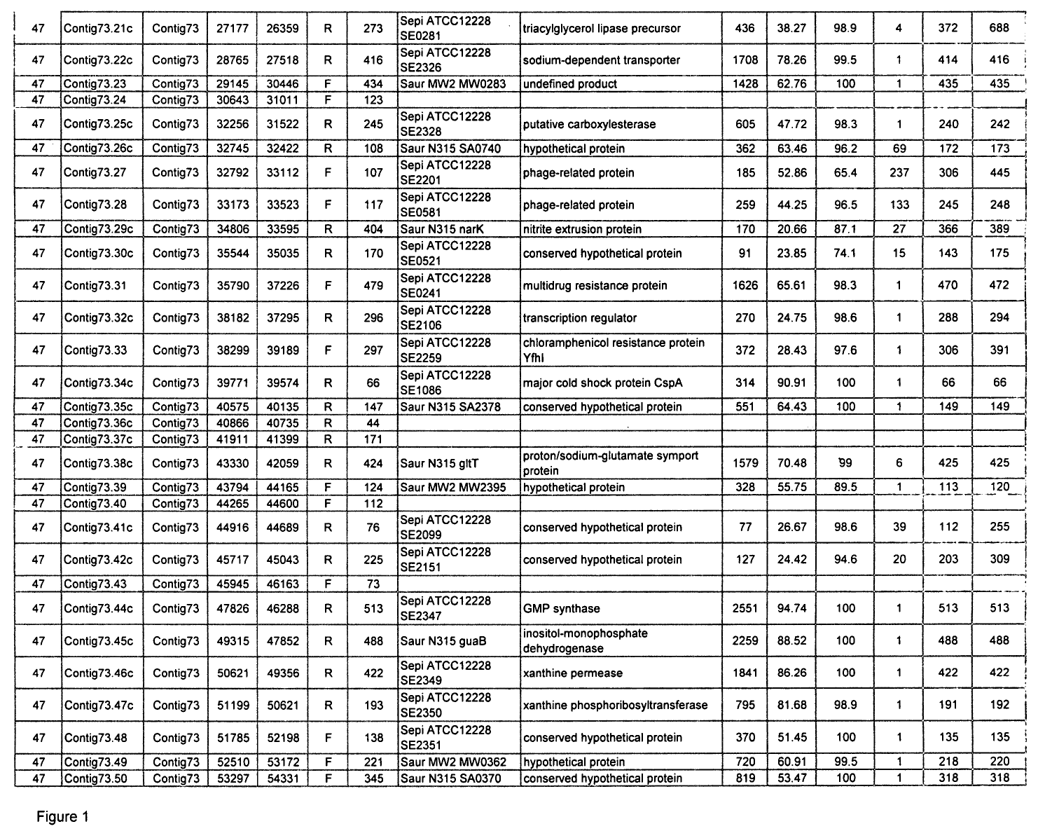

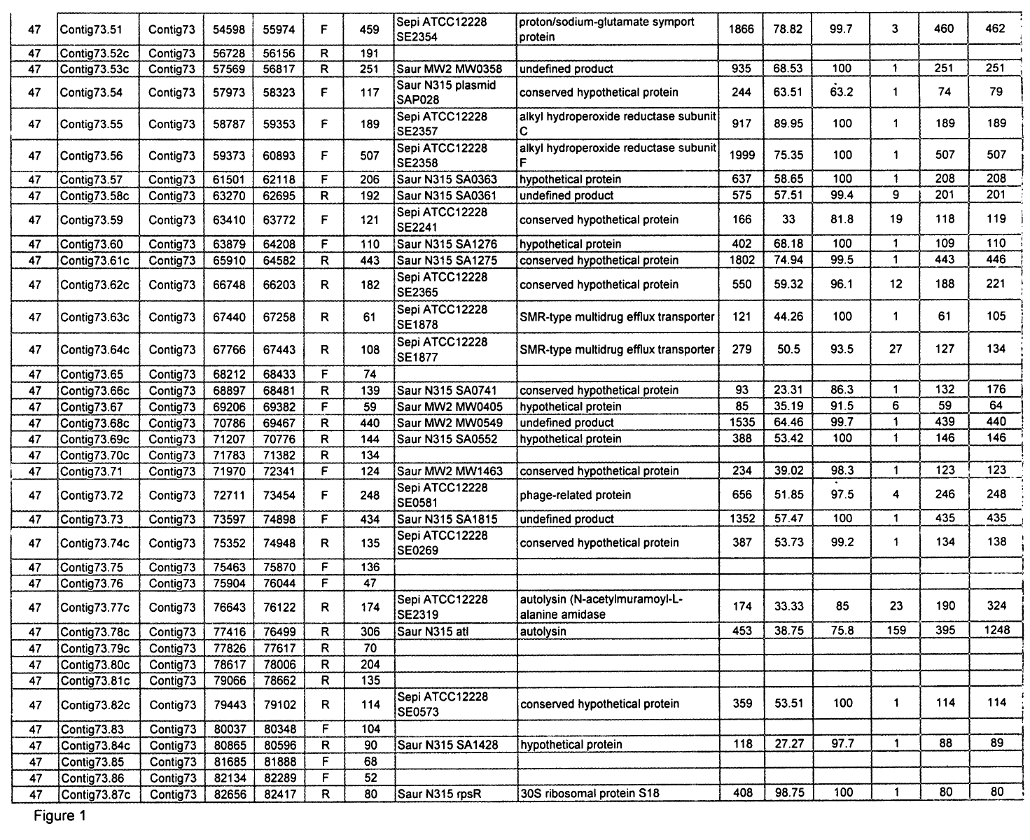

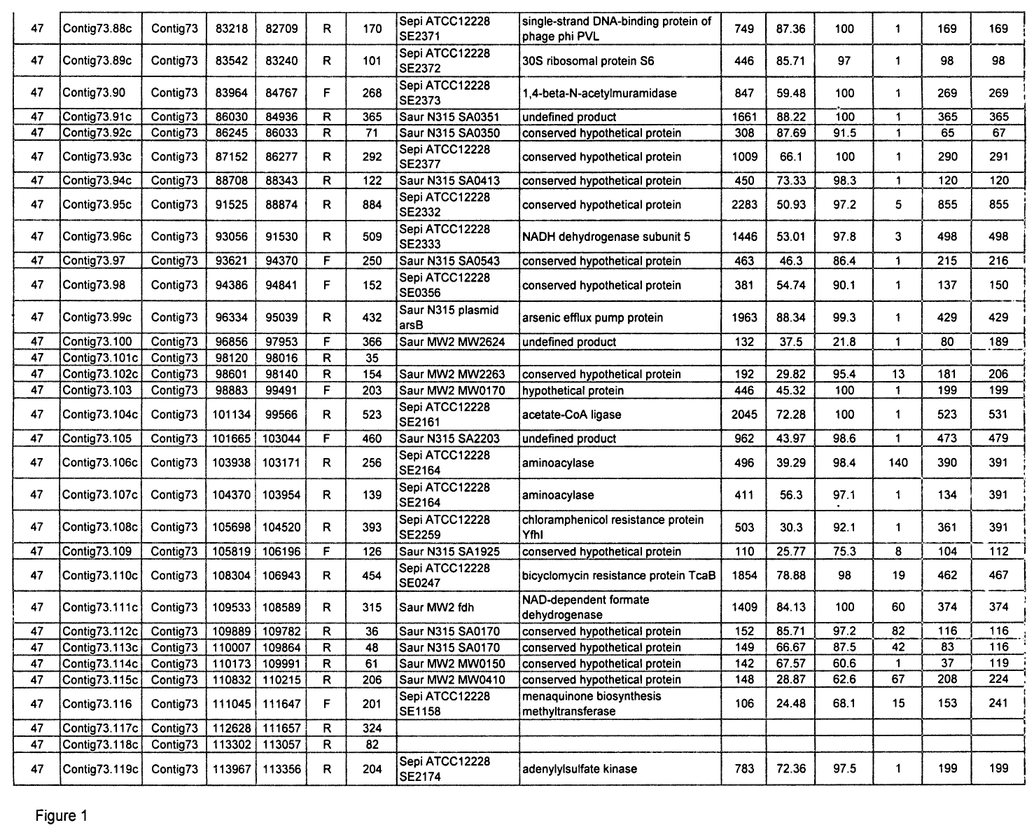

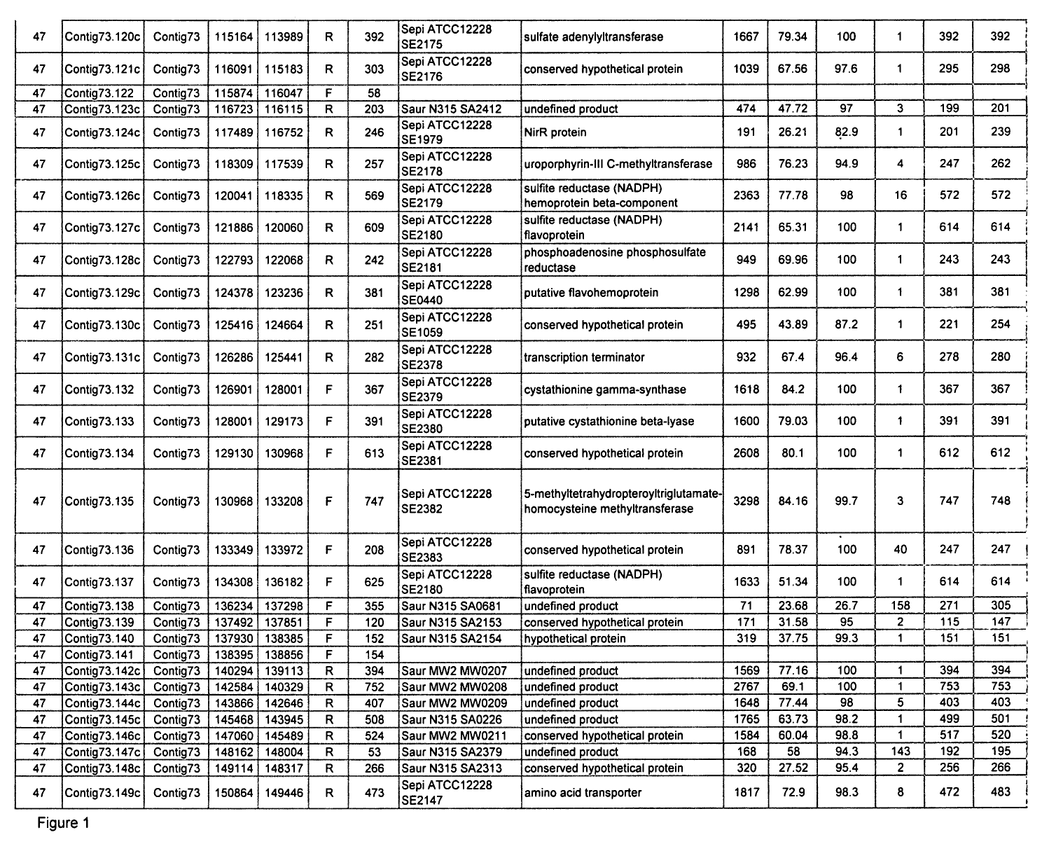

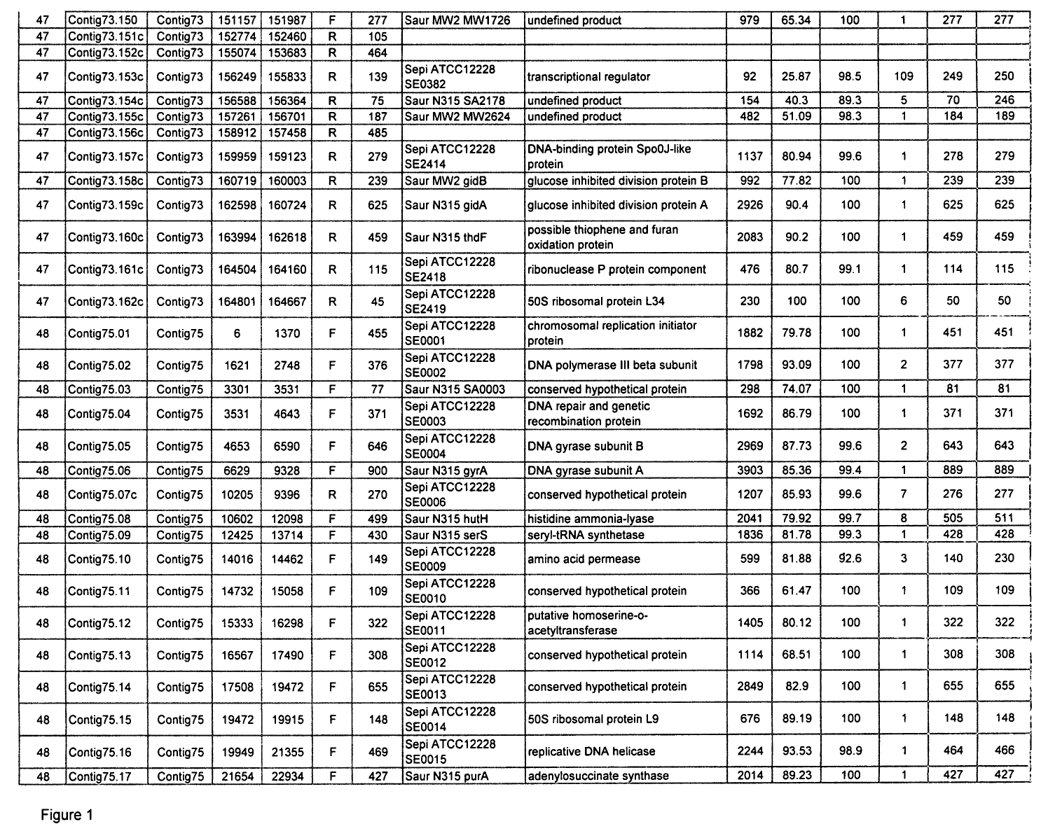

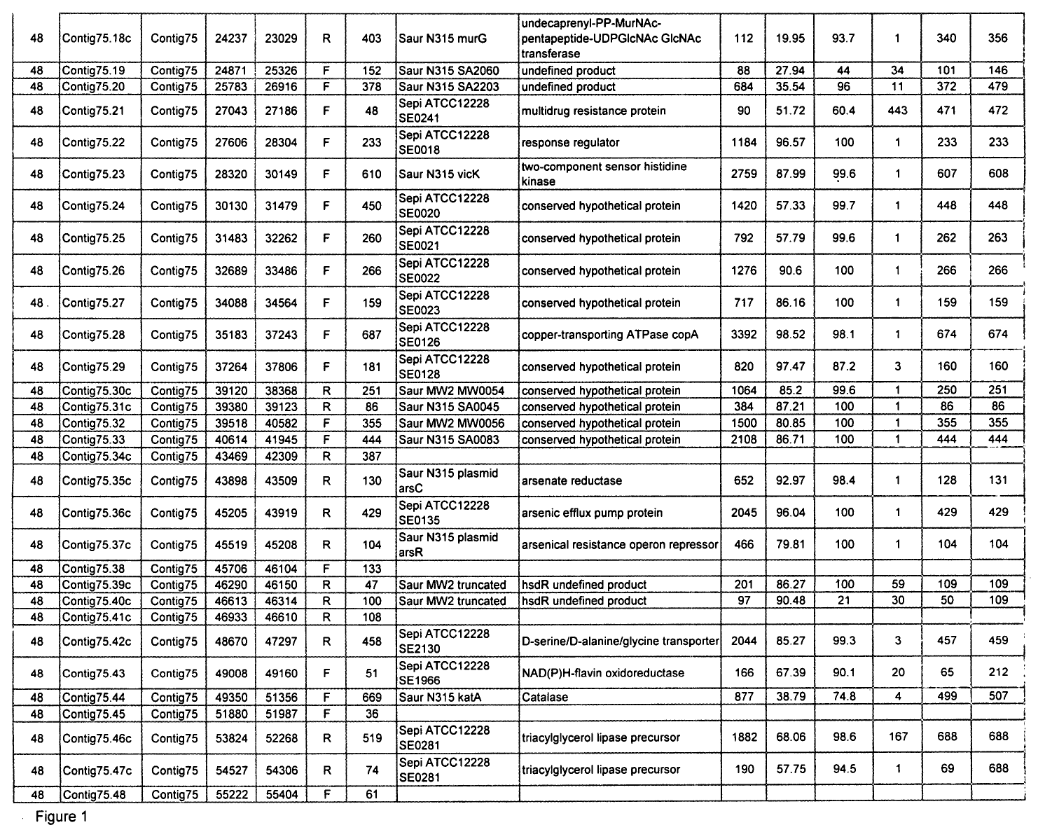

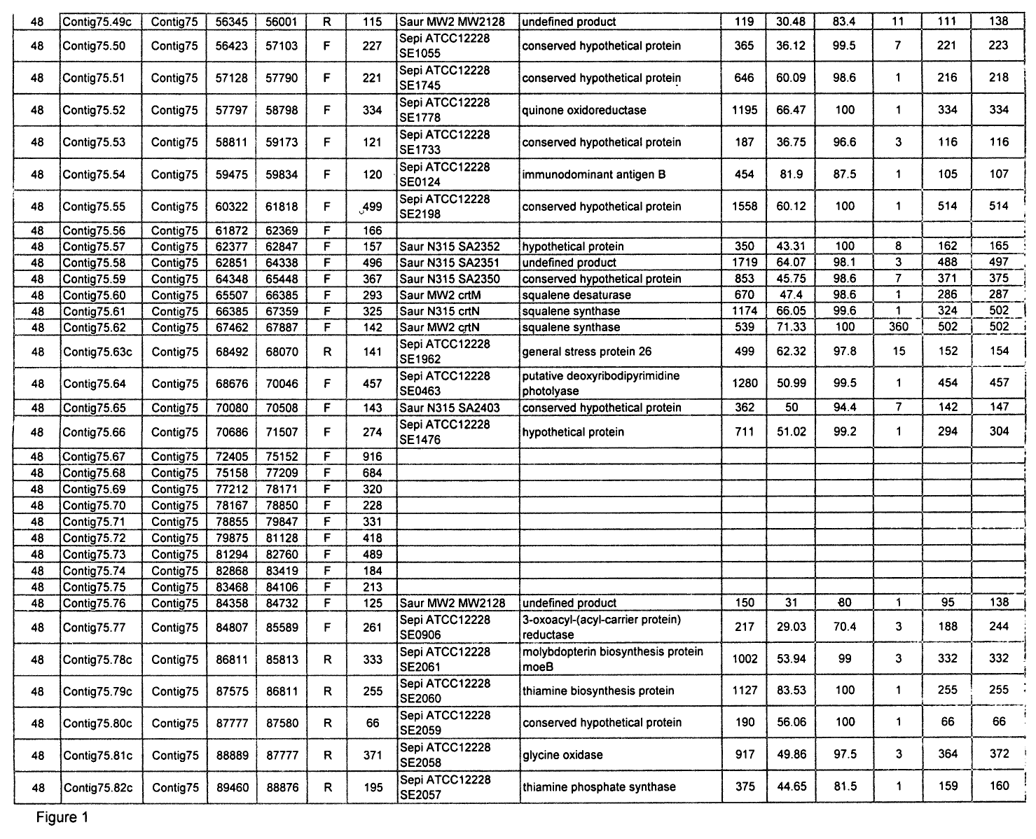

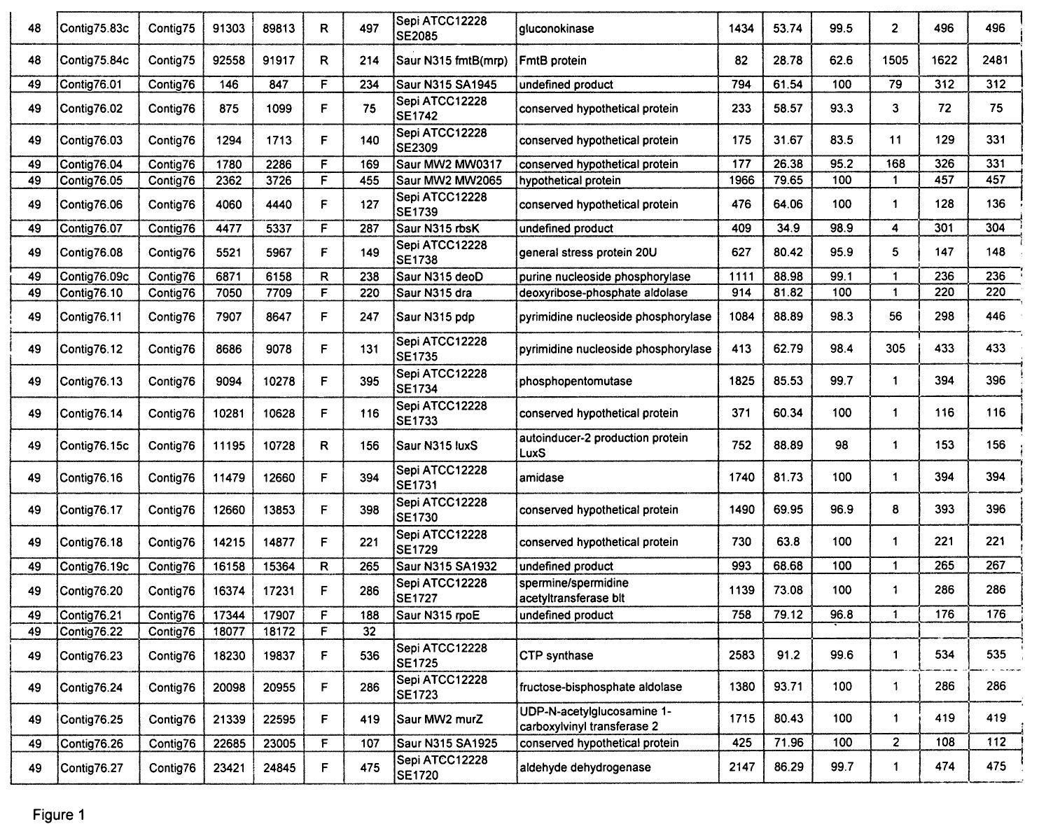

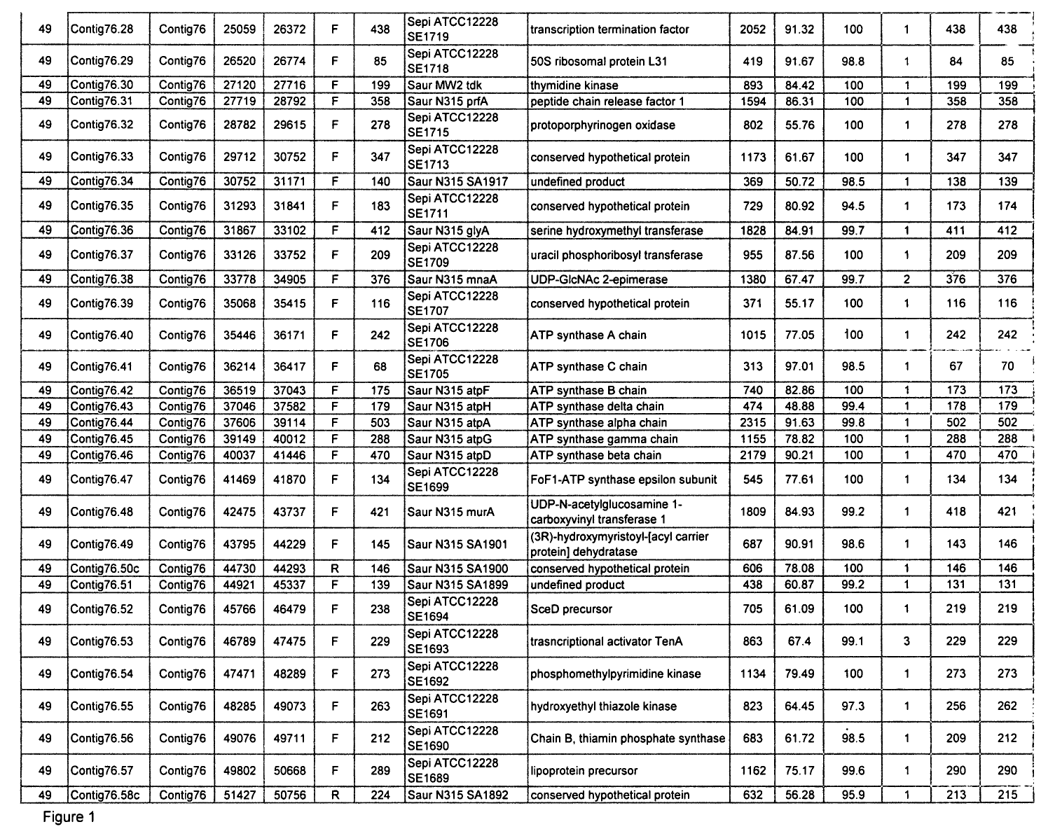

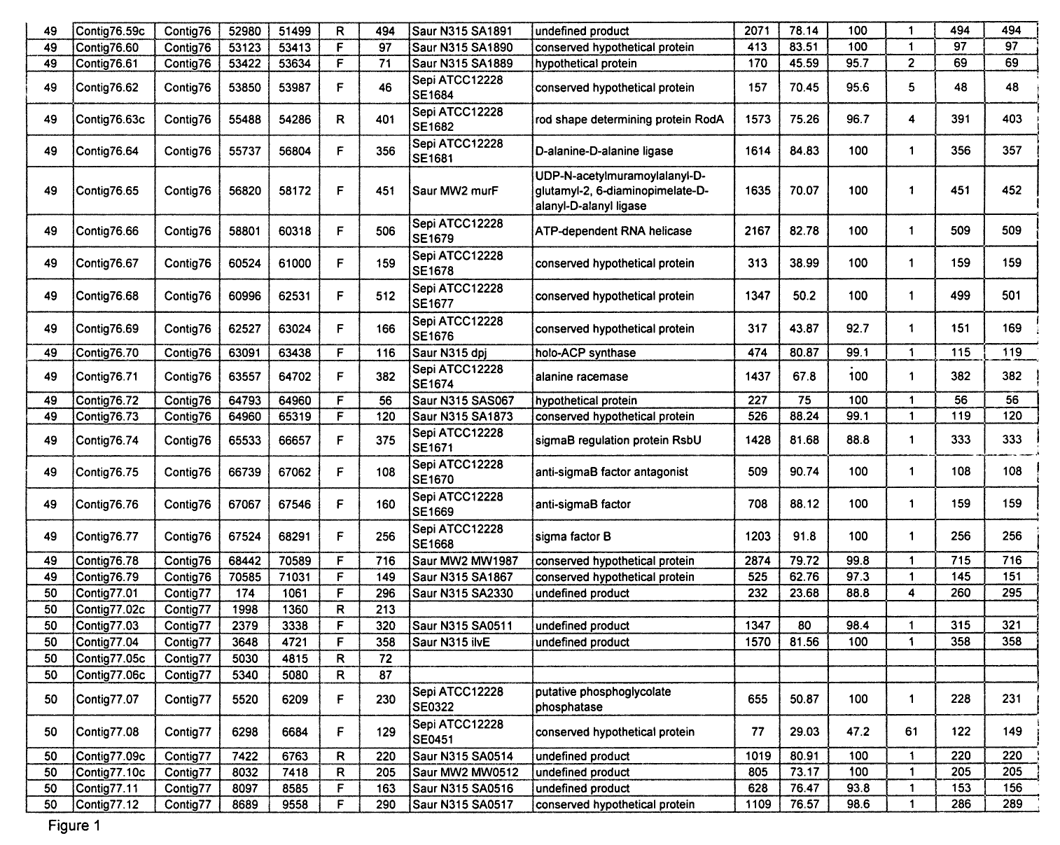

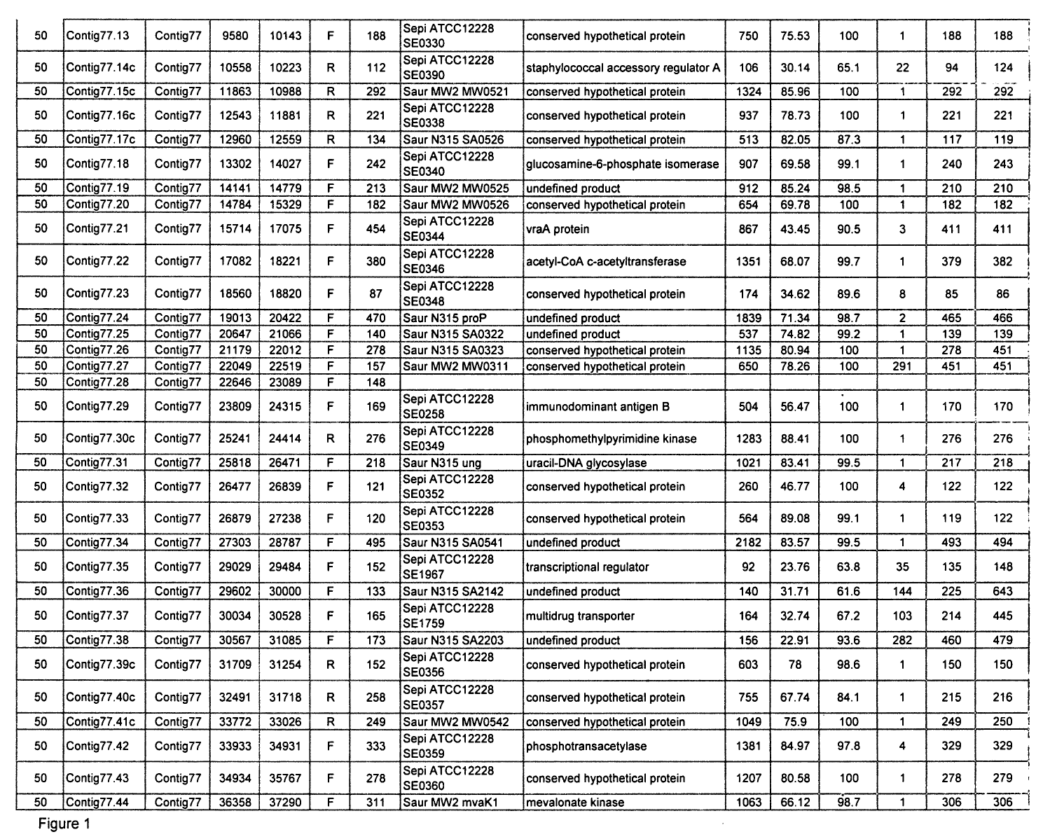

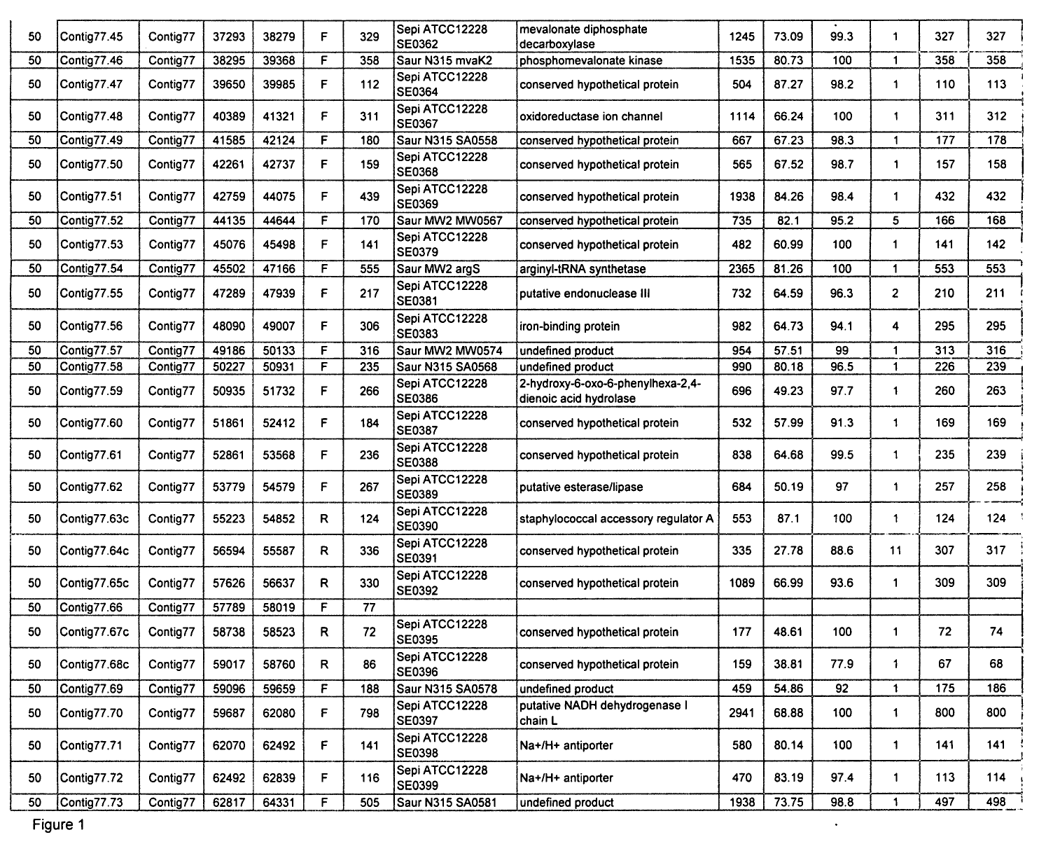

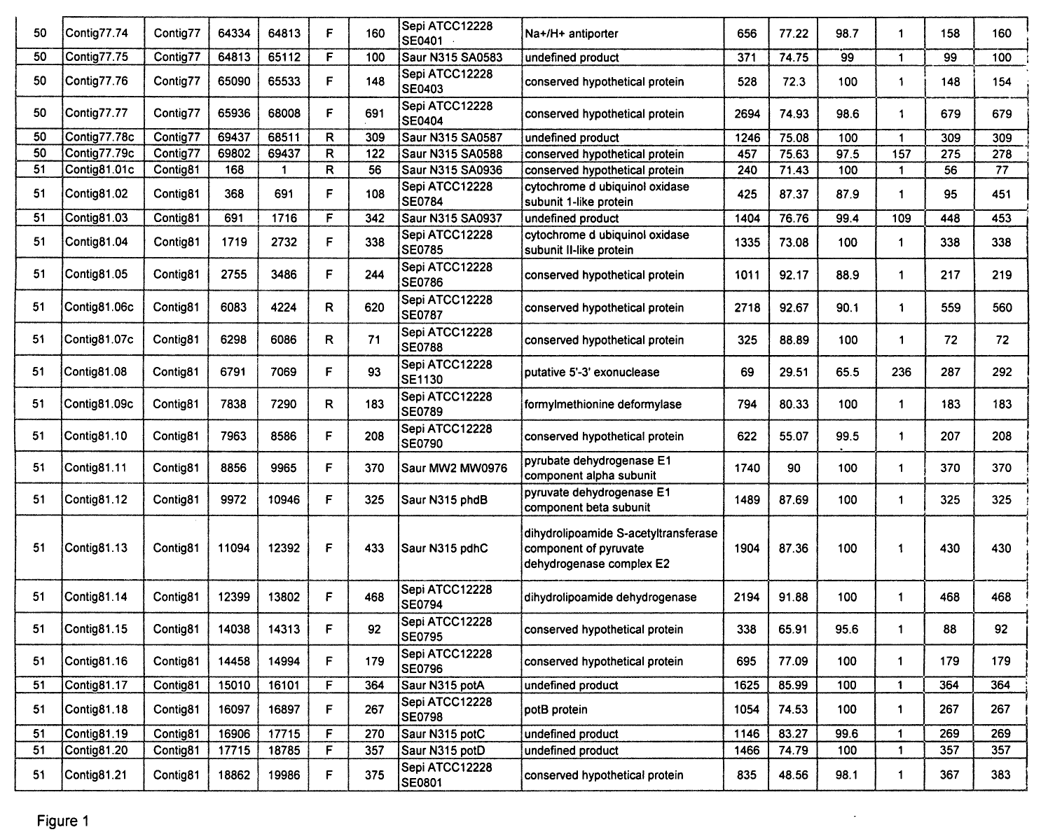

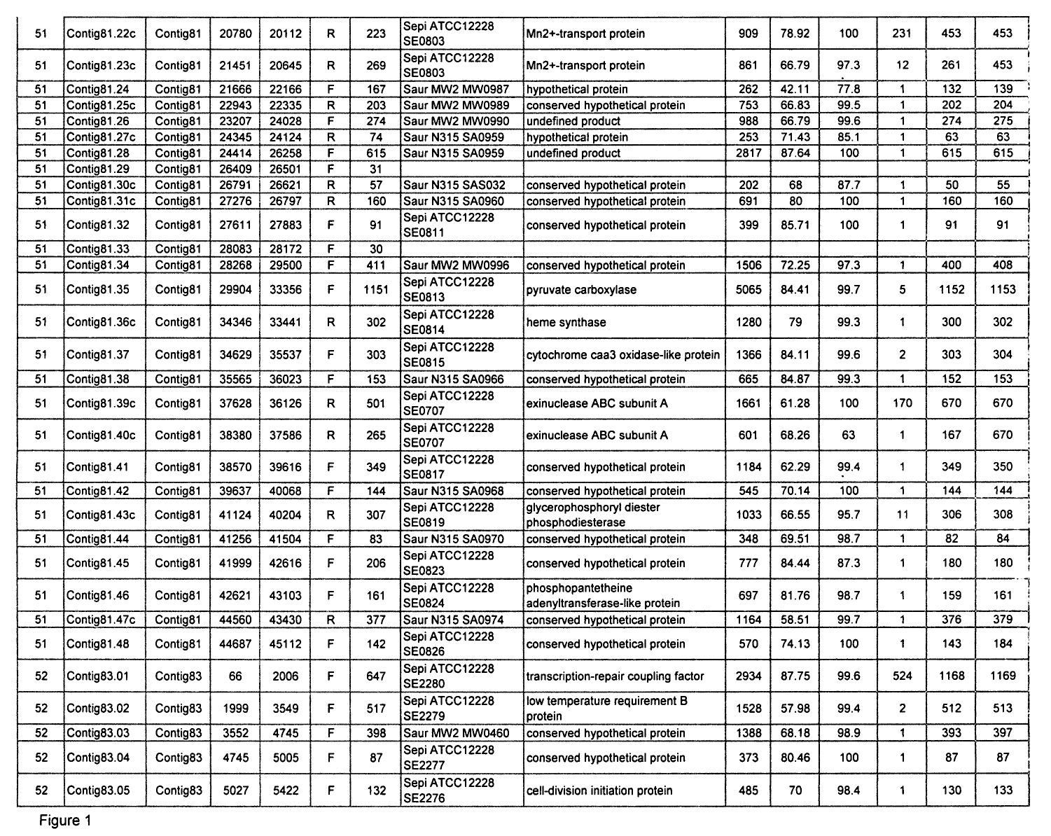

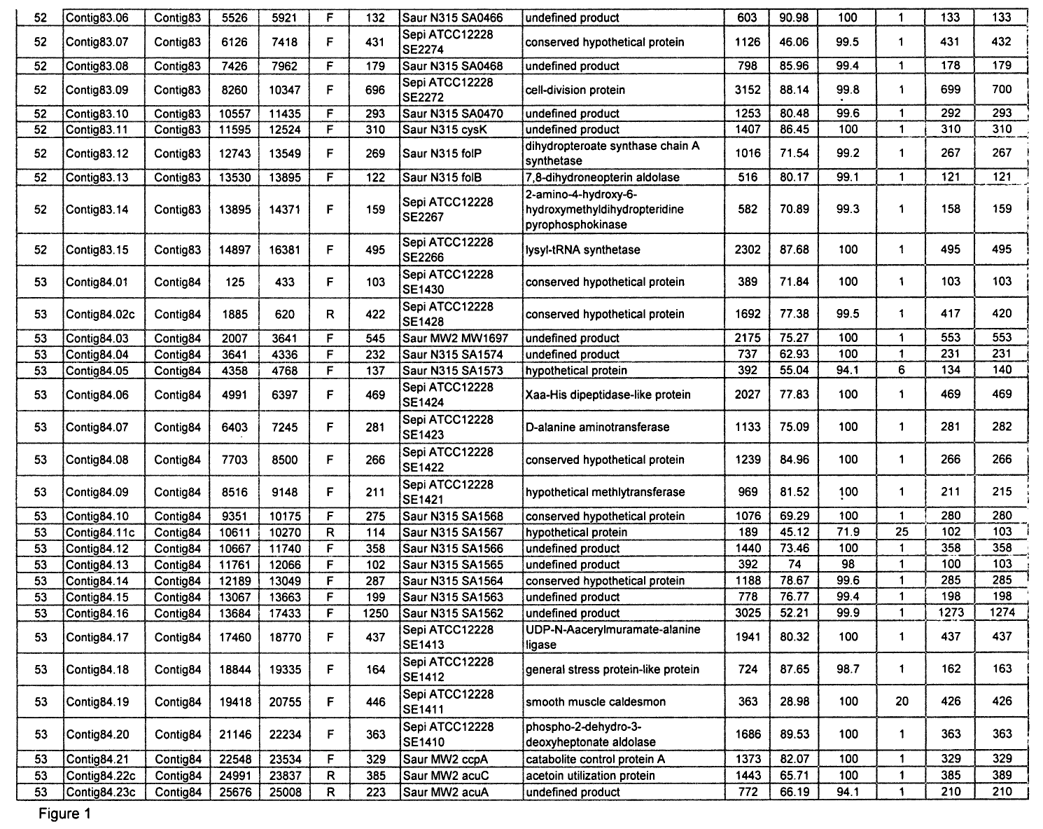

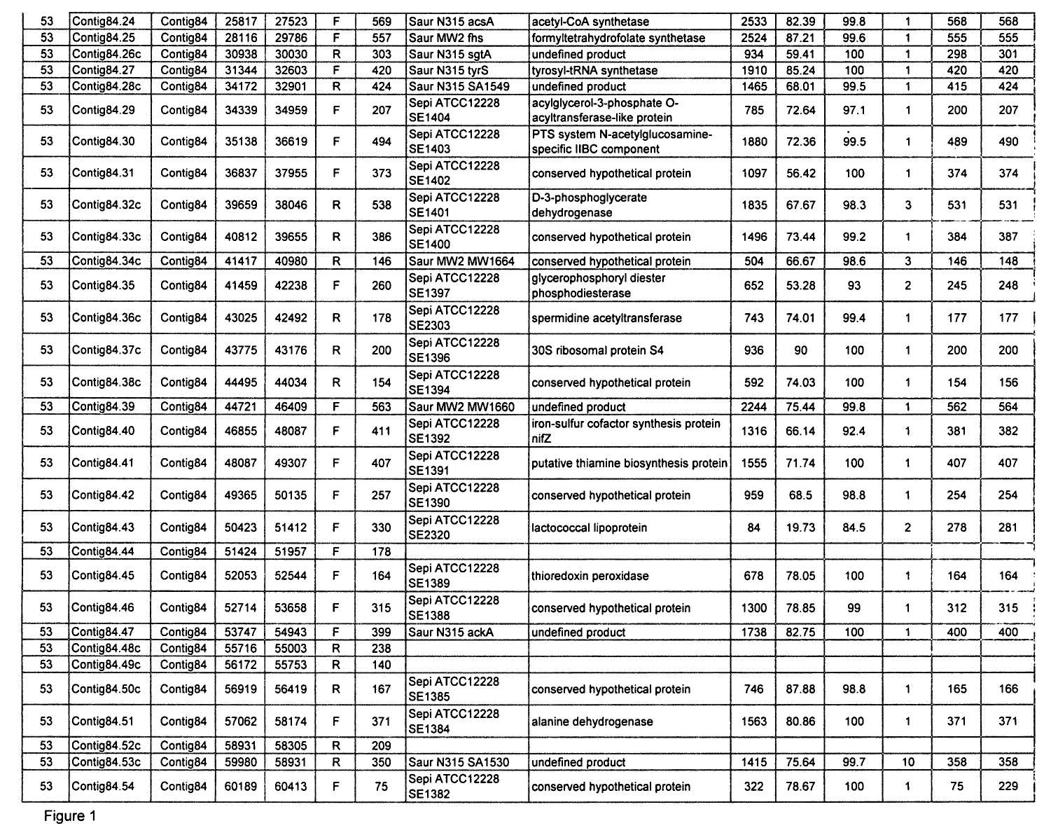

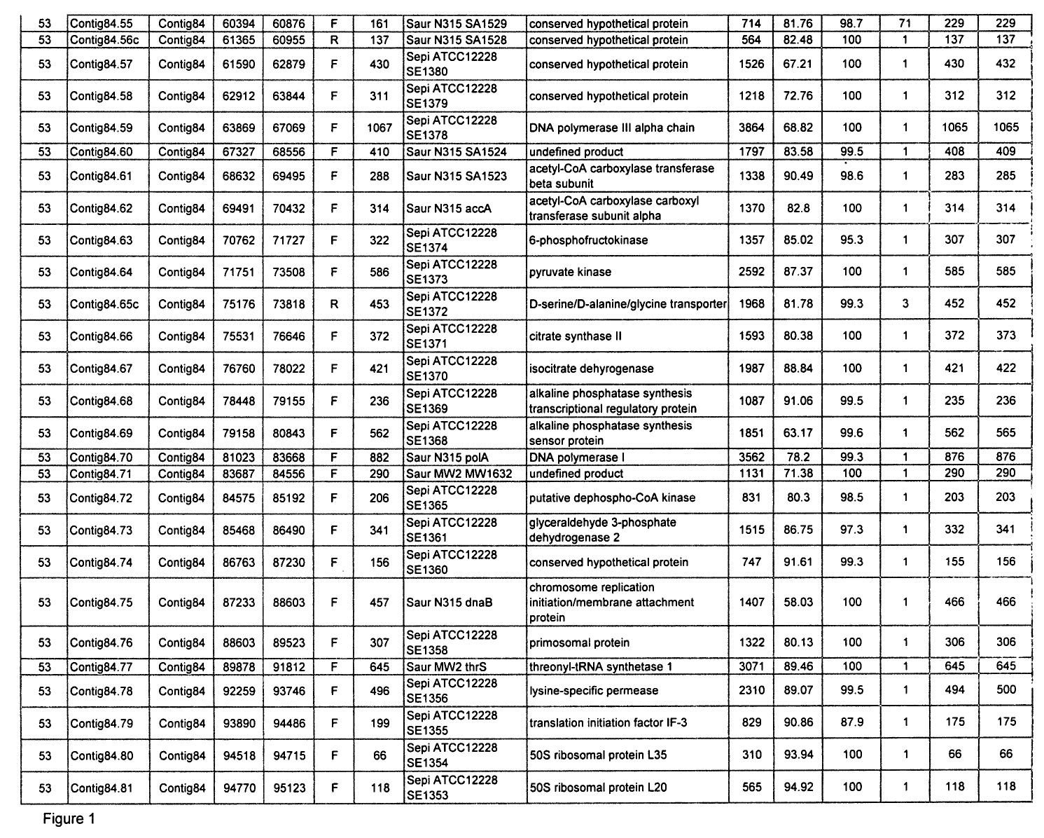

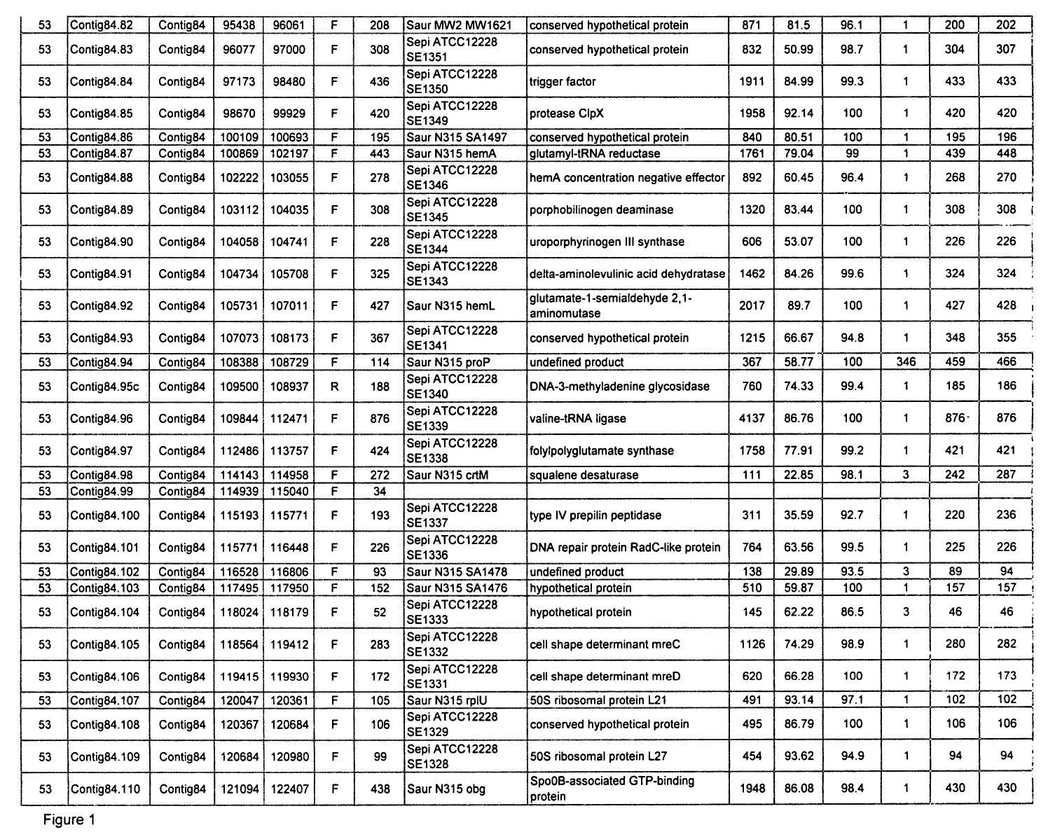

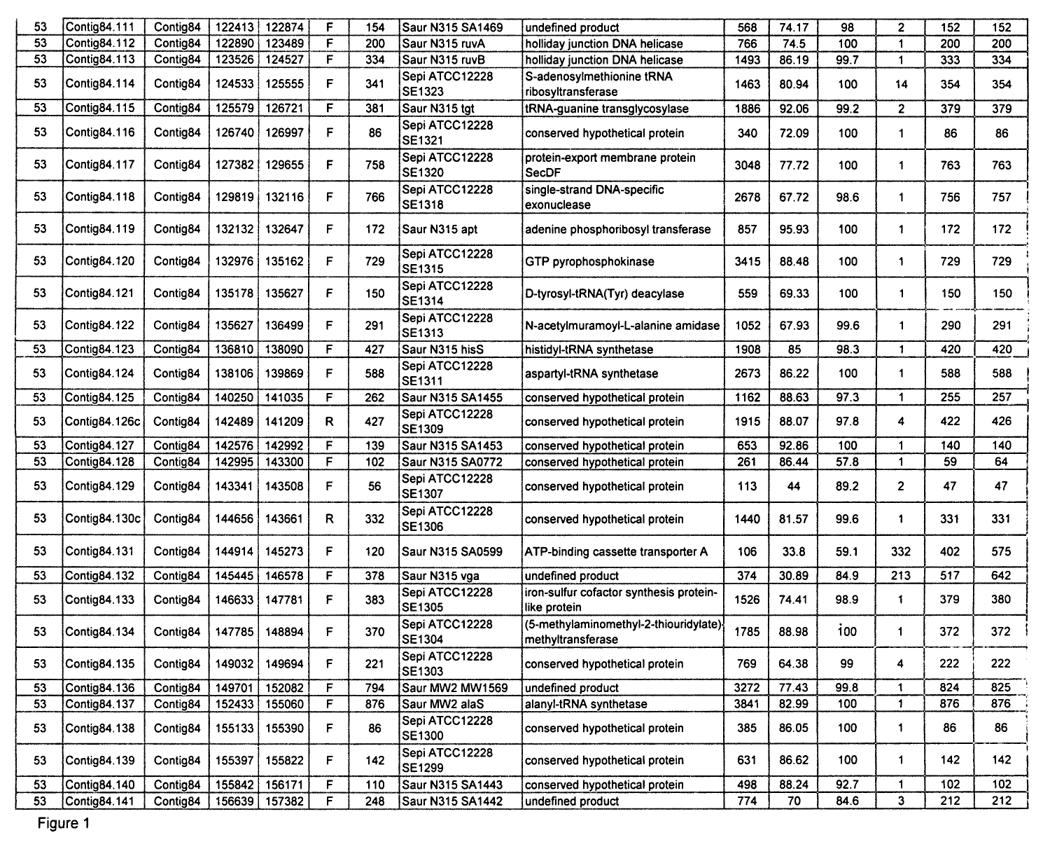

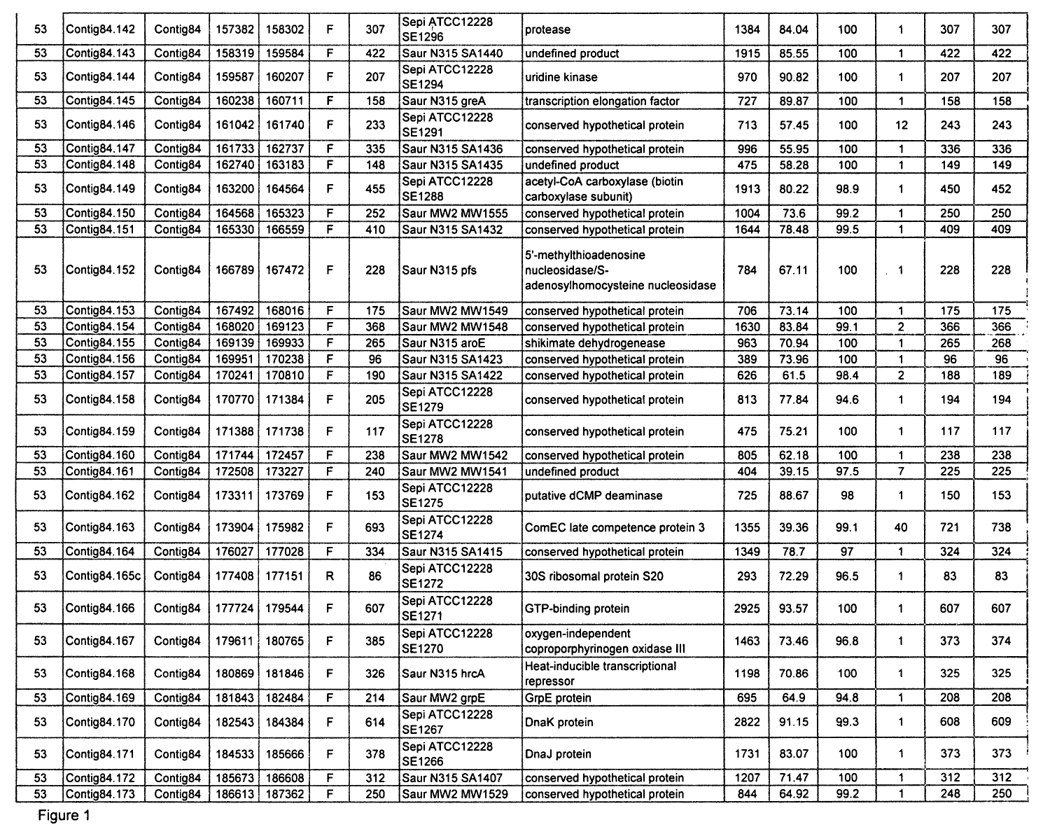

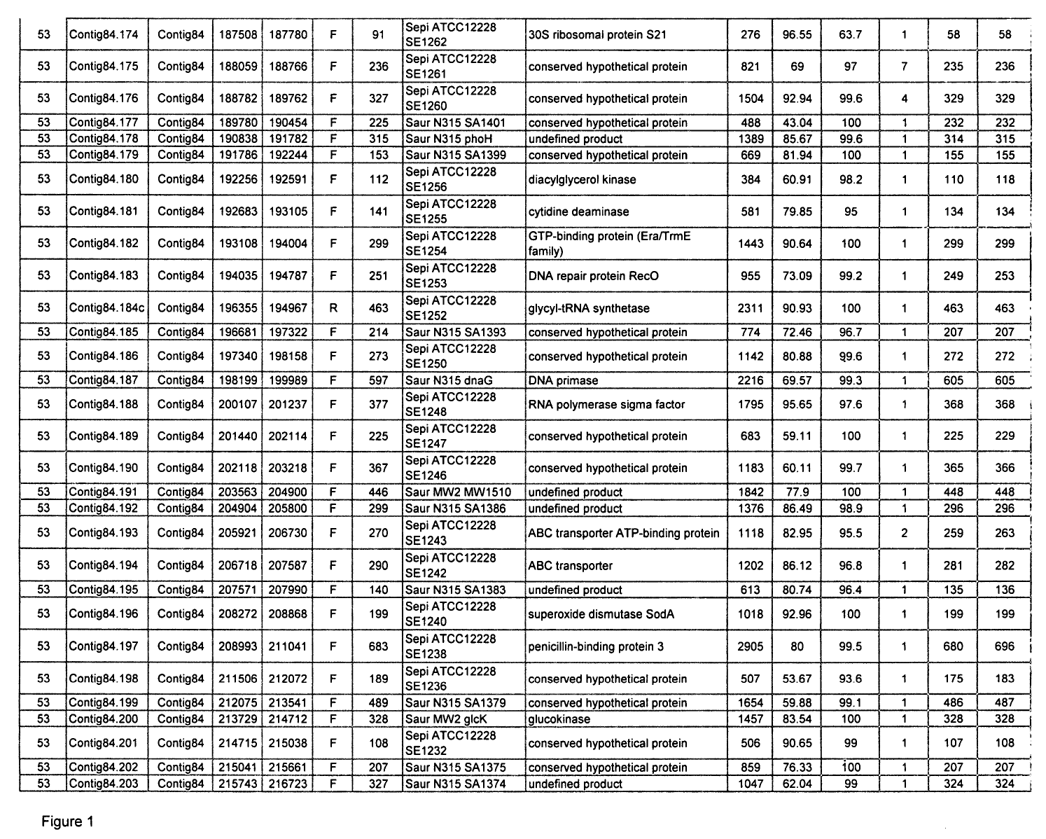

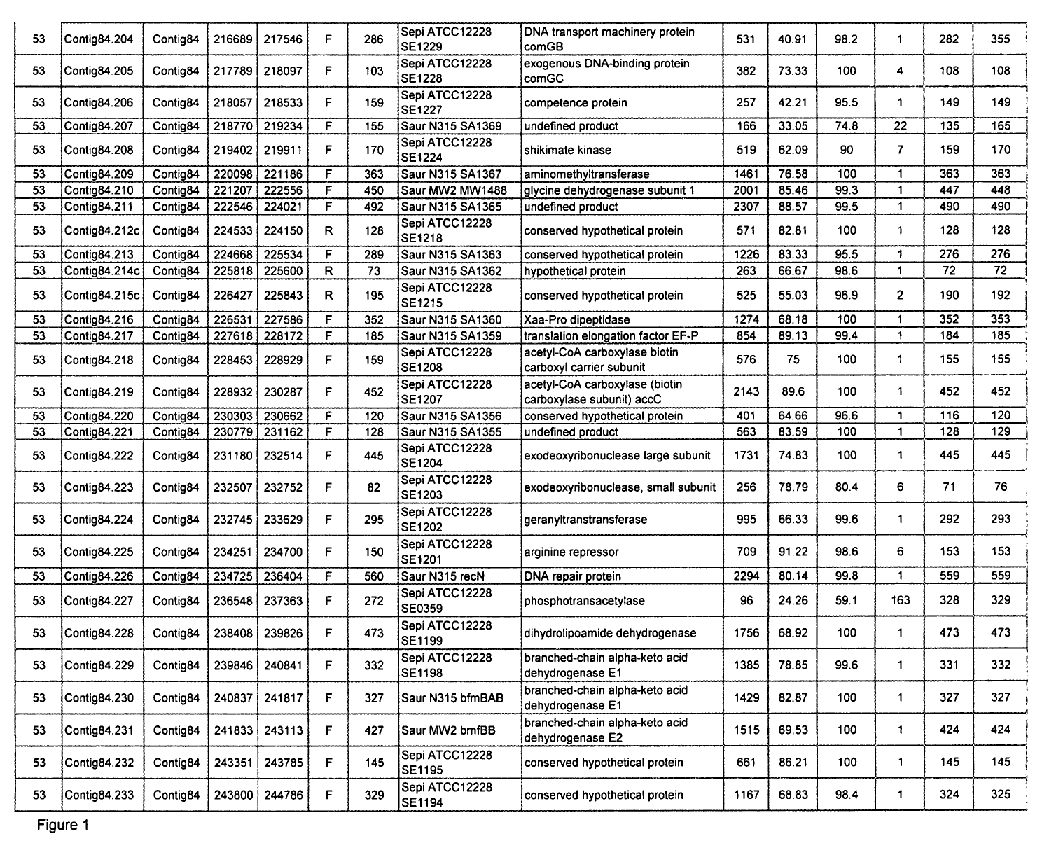

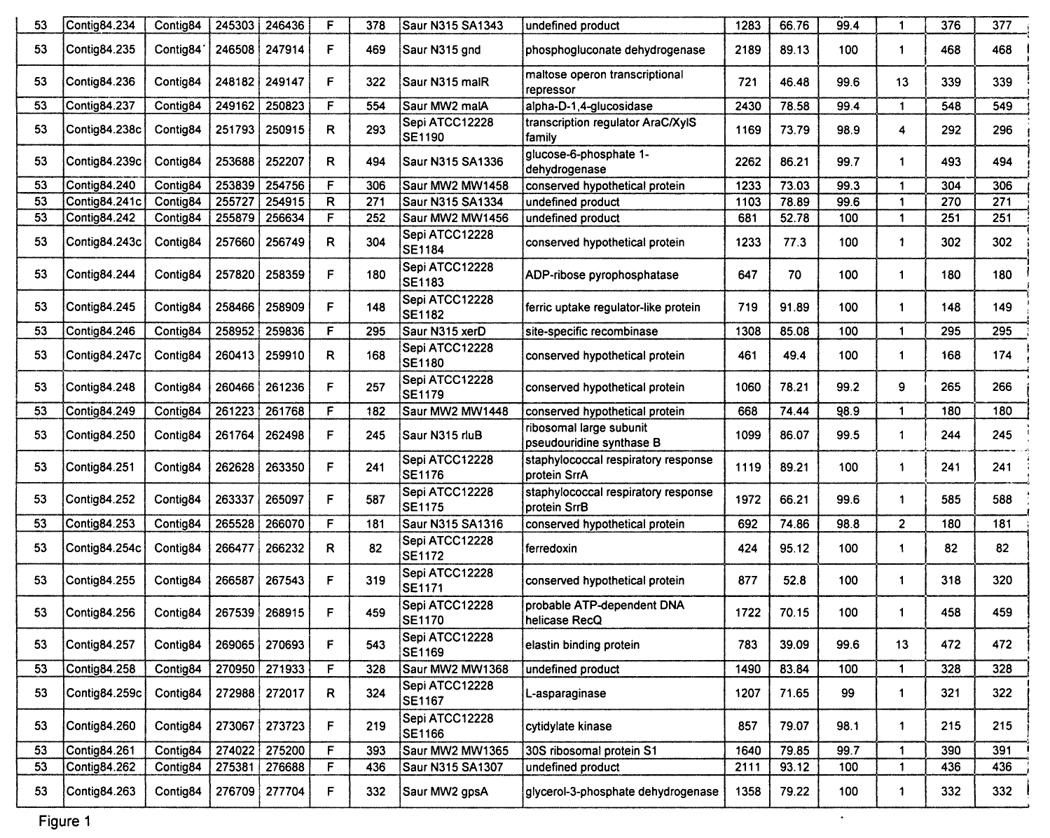

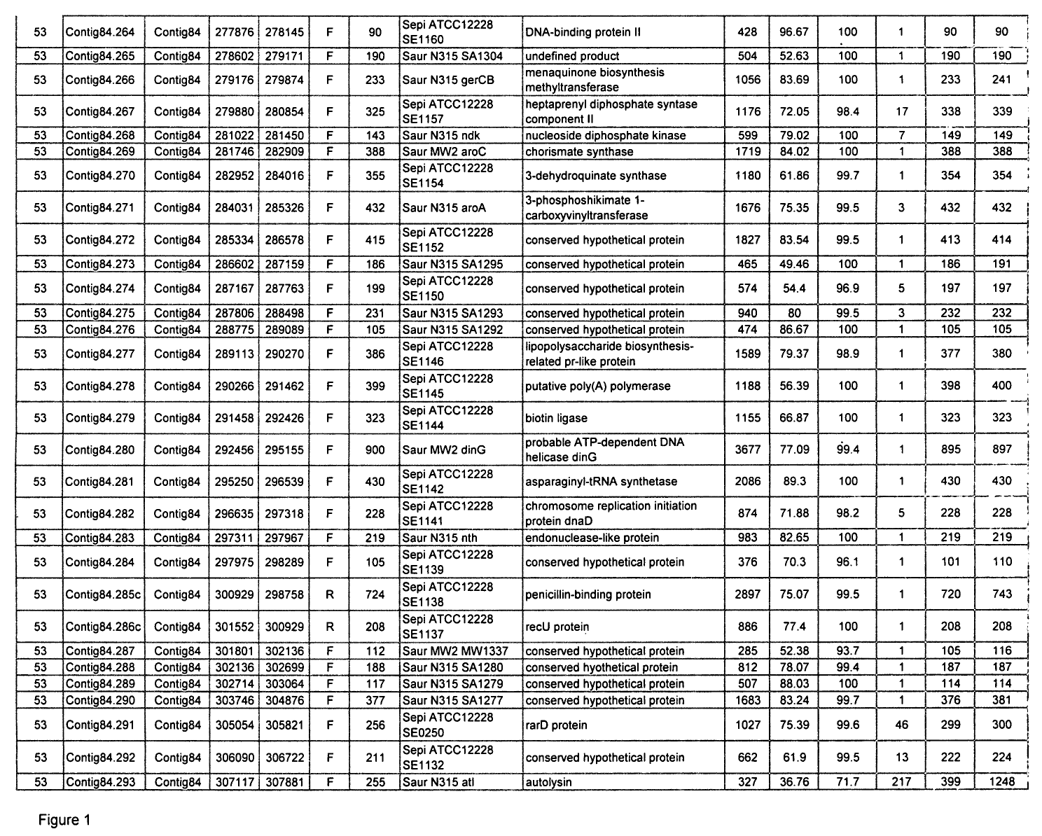

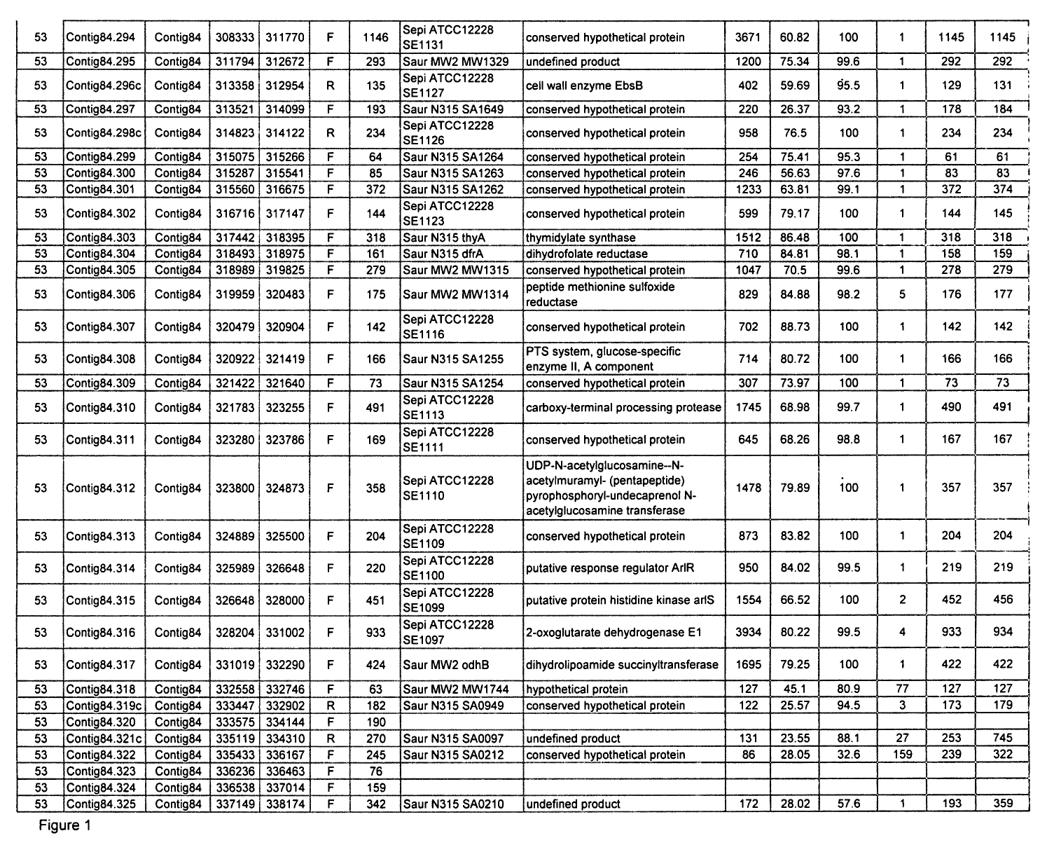

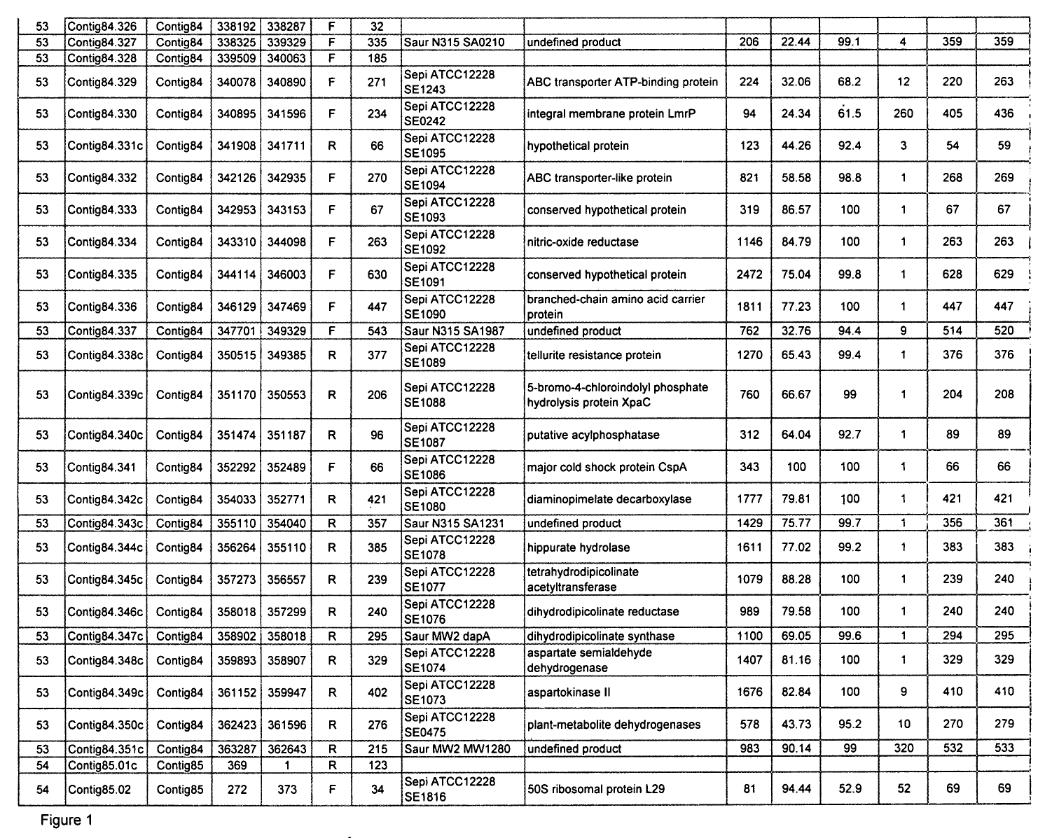

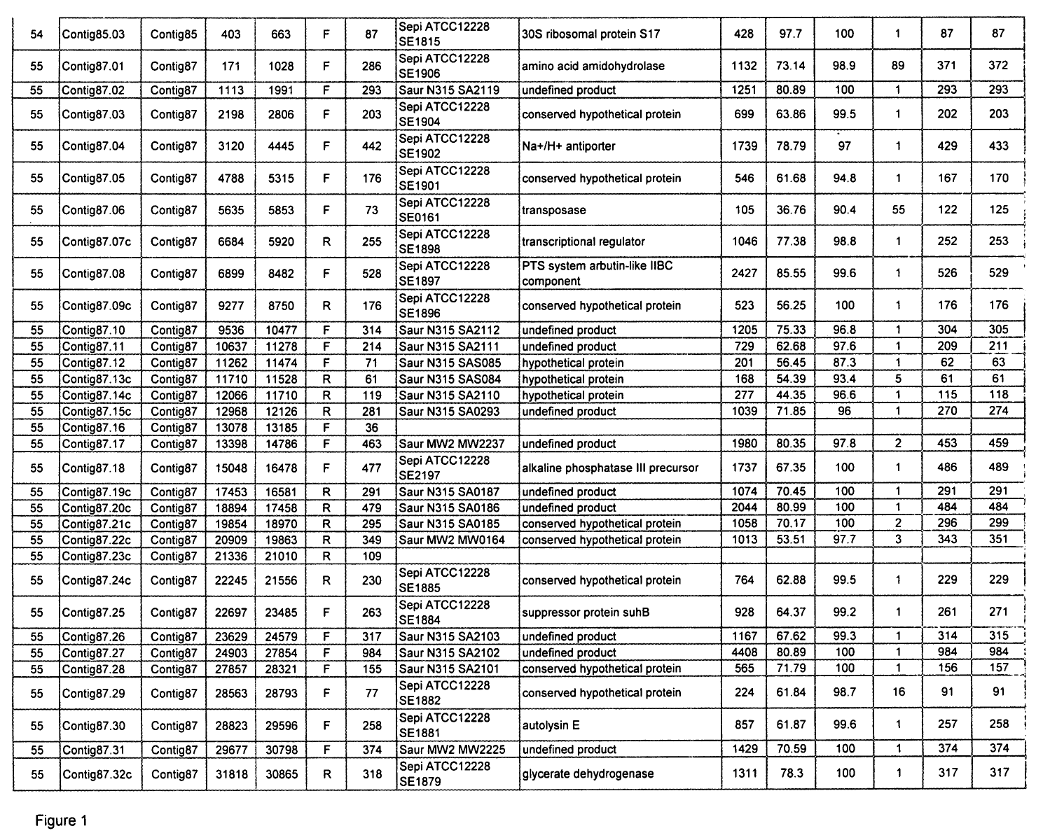

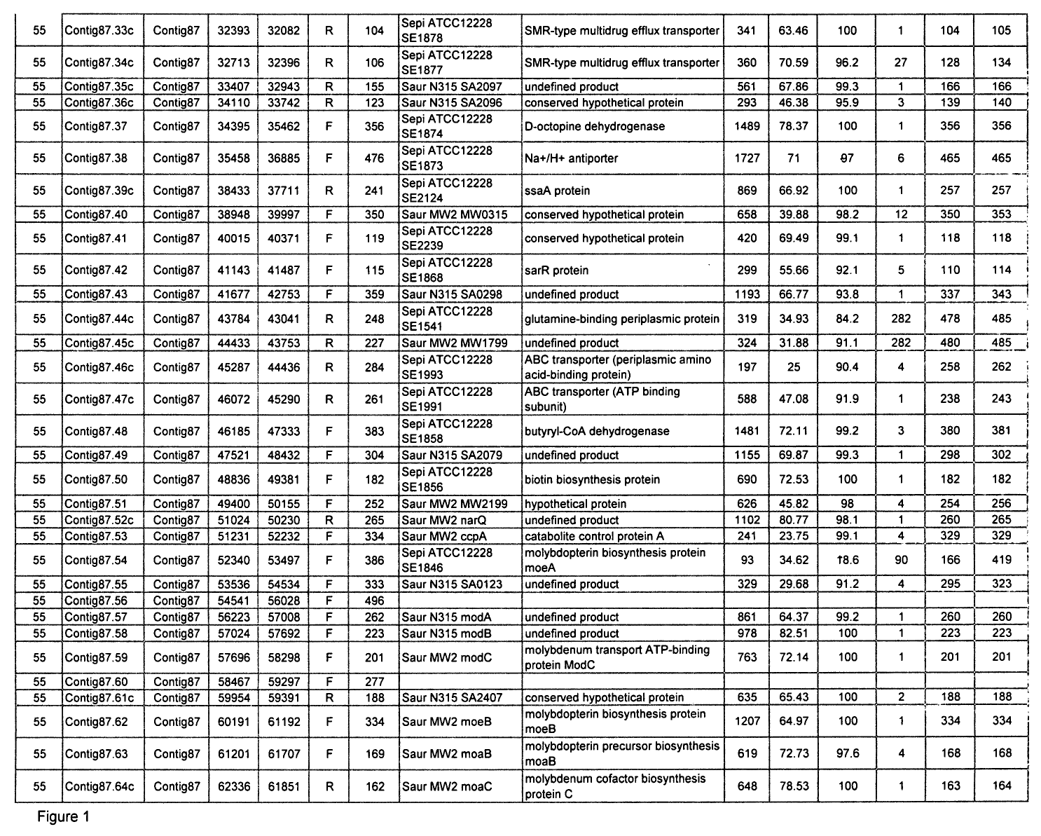

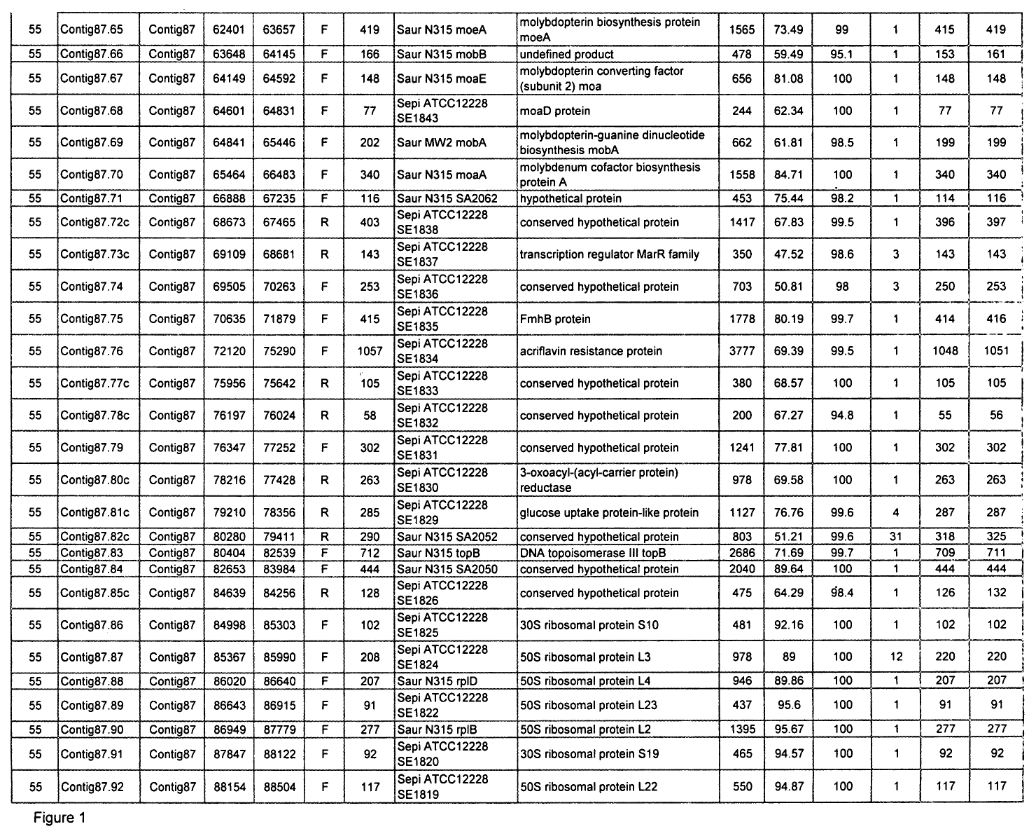

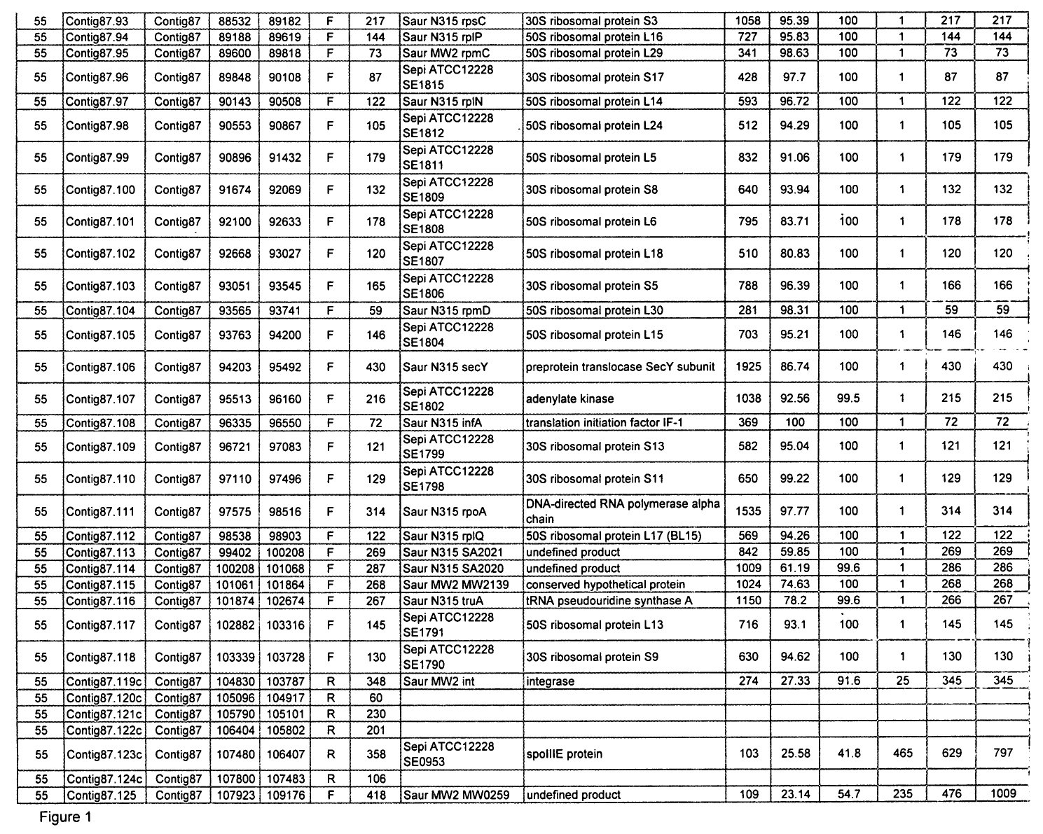

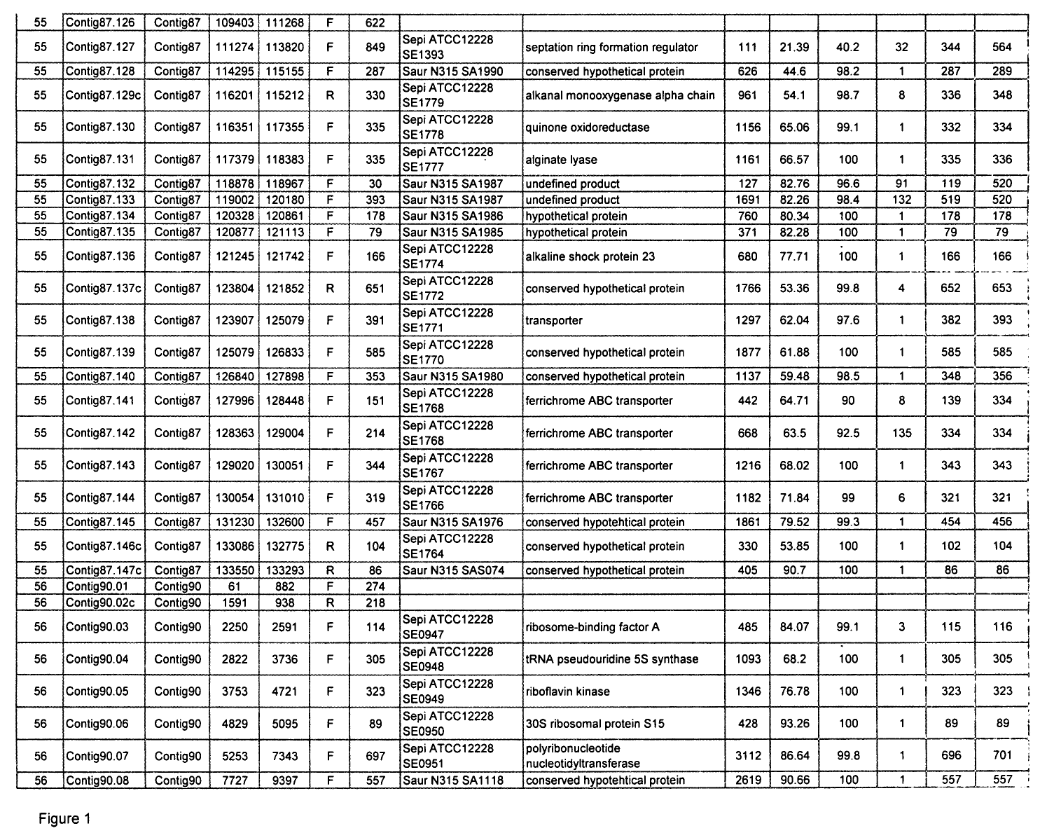

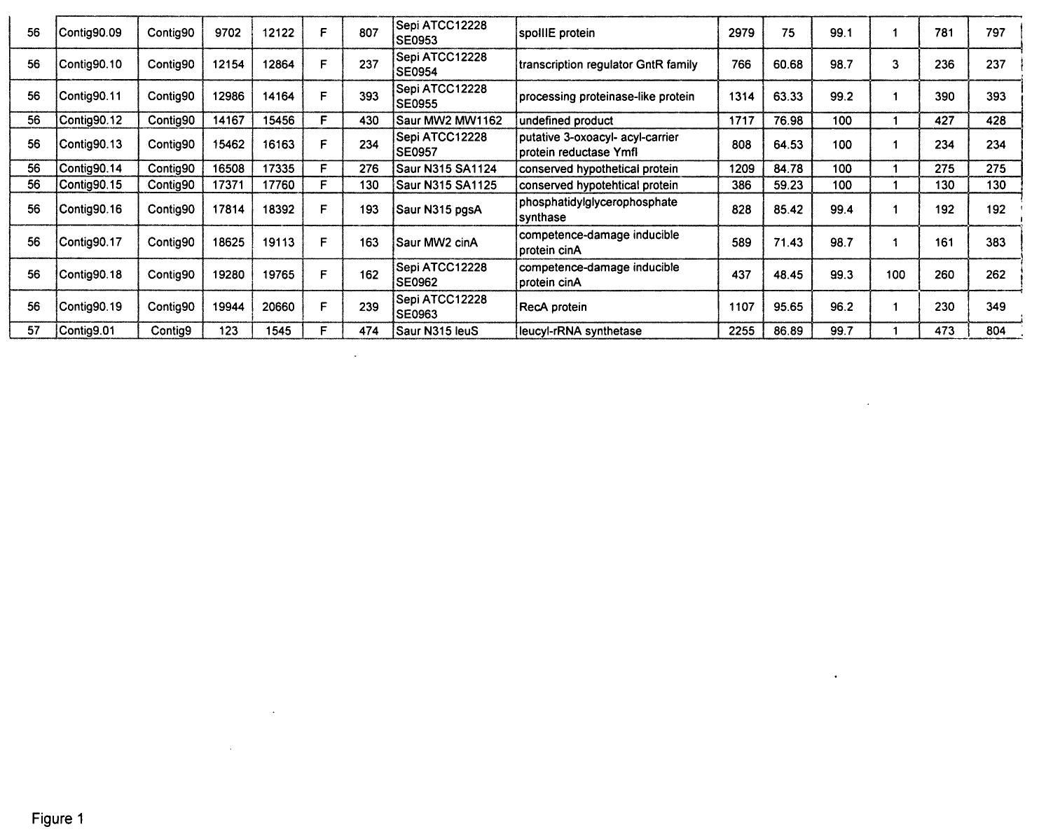

The present invention is based upon the sequencing and analysis of the genome of Staphylococcus saprophyticus. The invention features isolated nucleic acid molecules including nucleotide sequences within SEQ ID NO:1-57 as described in FIG. 1, which encode particular polypeptides. FIG. 1 details the nucleic acid sequences of interest, for example, the invention includes nucleotides 835-1164 of SEQ ID NO:1 which encodes a conserved hypothetical protein and nucleotides 1205-1535 of SEQ ID NO:1 which encodes a O-succinylbenzoic acid-CoA ligase. The start and stop nucleotides corresponding to the first and last nucleotide base pairs of the coding sequence are also set forth in FIG. 1.

Accordingly, the invention provides nucleic acid and polypeptide molecules from S. saprophyticus, a facultative anaerobic, gram positive bacteria which has been identified as a major cause of urinary tract infections, in addition to other diseases and disorders. One aspect of the invention features nucleic acid molecules which are useful e.g., for production of recombinant proteins useful e.g., for screening for inhibitors, e.g., small molecules, which are useful as prophylactic or therapeutic agents to treat or prevent S. saprophyticus-associated diseases or disorders, e.g., a urinary tract infection. The polypeptides of the invention can be formulated for use in compositions e.g., vaccines, to induce immune responses to treat or prevent S. saprophyticus-associated diseases or disorders. Moreover, the nucleic acid or polypeptide molecules of the invention may also be used as diagnostic agents to identify S. saprophyticus, e.g., in a subject, to thereby diagnose whether the subject has or is at risk for an S. saprophyticus-associated disease or disorder, e.g., a urinary tract infection. The present invention also includes probes and primers, which may be used, for example, to identify S. saprophyticus or to identify related bacterial species. In addition, the invention includes antisense molecules based on the nucleic acid molecules of the invention. The present invention also includes antibodies, or fragments thereof, against polypeptides of the invention, or fragments thereof.

The polypeptides encoded by the nucleic acid sequences within SEQ ID NO:1-57 can be easily derived from FIG. 1 and can be readily determined by one skilled in the art. As noted above, the start and stop nucleotides corresponding to the first and last nucleotide base pairs of the coding sequence are set forth in FIG. 1 and one skilled in the art using the genetic code could predict the corresponding amino acid sequence.

Thus, using FIG. 1 one skilled in the art could determine the amino acid sequence of each of the polypeptides encoded by the sequences presented in FIG. 1. For example, the nucleic acid sequence having nucleotides 4653-6590 of SEQ ID NO:48 encodes a DNA gyrase subunit B polypeptide having the amino acid sequence of SEQ ID NO: 58. Similarly, from FIG. 1, the nucleic acid sequence having nucleotides 6629-9328 of SEQ ID NO:48 encodes a predicted DNA gyrase subunit A polypeptide having the amino acid sequence of SEQ ID NO: 59; the nucleic acid sequence of 17460-18770 of SEQ ID NO:53 encodes a predicted UDP-N-Aacerylmuramate-alanine ligase having the amino acid sequence of SEQ ID NO: 60; the nucleic acid sequence of 20792-2151 1 of SEQ ID NO:31 encodes a predicted polypeptide of uridylate kinase having the amino acid sequence of SEQ ID NO:61; the nucleic acid sequence of 5432-4182 of SEQ ID NO:26 encodes a predicted polypeptide of UDP-N-acetylglucosamine pyrophophorylase having the amino acid sequence of SEQ ID NO: 62; and the nucleic acid sequence of 219402-219911 of SEQ ID NO:53 encodes a predicted shikimate kinase having the amino acid sequence of SEQ ID NO: 62.

For each S. saprophyticus polypeptide of the invention, an identification number for a polypeptide from another Staphylococcus species, e.g., an S. epidermidis strain or an S. aurelius strain, which has the highest percent identity to the polypeptide of the invention is also listed in FIG. 1. The S. epidermidis strain from which polypeptides were identified for FIG. 1 is strain ATCC 12228 (Genbank Accession No. NC_{—}004461). The S. aurelius strain from which polypeptides were identified for FIG. 1 is strain N315 (Genbank Accession No. NC_{—}002745), and the S. aureliusNW2 strain from which polypeptides were identified for FIG. 1 is Genbank Accession No. NC_{—}003923.

The precent identity to a particular polypeptide of the invention to a known protein can be readily determined using FIG. 1. For example, a D-serine/D-alanine/glycine transporter was identified in SEQ ID NO:7 (Contig 19.02c). This polypeptide was identified to have 67.41% identity to S. epidermidis ID number SE1372.

The nucleotide and amino acid sequences of the S. epidermidis and S. aurelius genes and proteins listed in FIG. 1 are publicly available at, for example, the National Center for Biotechnology Information, PubMed website using the identification numbers listed in FIG. 1.

S. epidermidis and S. aurelius are facultative anaerobic, gram positive bacteria which are closely related to S. saprophyticus. Based on the similarity between S. epidermidis, S. aurelius and S. saprophyticus, the function of the polypeptides of the invention may be determined based on homology to closely related S. epidermidis and S. aurelius polypeptides.

An “S. saprophyticus-associated disease or disorder,” as used herein, includes any disease or disorder caused by or related to infection with S. saprophyticus. For example, an S. saprophyticus disease or disorder includes, but is not limited to, urinary tract infection (UTI; also referred to as cystitis), or symptoms associated therewith. Urinary tract infection is a common infection which is usually caused when bacteria, e.g., S. saprophyticus, enter the urethra and bladder and cause inflammation and infection. Kidney infection (pyelonephritis) may result from urinary tract infection.

An “open reading frame”, also referred to herein as ORF, is a region of nucleic acid which encodes a polypeptide. This region may represent a portion of a coding sequence or a total sequence and can be determined from a stop to stop codon or from a start to stop codon.

As used herein, a “coding sequence” is a nucleic acid which is transcribed into messenger RNA and/or translated into a polypeptide when placed under the control of appropriate regulatory sequences. The boundaries of the coding sequence are determined by a translation start codon at the five prime terminus and a translation stop code at the three prime terminus. A coding sequence can include but is not limited to RNA, e.g., messenger RNA, synthetic DNA, and recombinant nucleic acid sequences.

A “complement” of a nucleic acid as used herein refers to an anti-parallel or antisense sequence that participates in Watson-Crick base-pairing with the original sequence.

A “gene product” is a protein or structural RNA which is specifically encoded by a gene.

As used herein, the term “probe” refers to a nucleic acid, peptide or other chemical entity which specifically binds to a molecule of interest. Probes are often associated with or capable of associating with a label. A label is a chemical moiety capable of detection. Typical labels comprise dyes, radioisotopes, luminescent and chemiluminescent moieties, fluorophores, enzymes, precipitating agents, amplification sequences, and the like. Similarly, a nucleic acid, peptide or other chemical entity which specifically binds to a molecule of interest and immobilizes such molecule is referred herein as a “capture ligand”. Capture ligands are typically associated with or capable of associating with a support such as nitro-cellulose, glass, nylon membranes, beads, particles and the like. The specificity of hybridization is dependent on conditions such as the base pair composition of the nucleotides, and the temperature and salt concentration of the reaction. These conditions are readily discernable to one of ordinary skill in the art using routine experimentation.

The term “homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

The terms peptides, proteins, and polypeptides are used interchangeably herein.

A polypeptide has S. saprophyticus biological activity if it has one, two and preferably more of the following properties: (1) any activity related to or associated with the initiation or progression of infection or disease, e.g., in a subject; (2) if when expressed in the course of an S. saprophyticus infection, it can promote, or mediate the attachment of S. saprophyticus to a cell; (3) it has an enzymatic activity, structural or regulatory function characteristic of an S. saprophyticus protein; (4) the gene which encodes it can rescue a lethal mutation in an S. saprophyticus gene; (5) or it is immunogenic in a subject. A polypeptide has biological activity if it is an antagonist, agonist, or super-agonist of a polypeptide having one of the above-listed properties.

A biologically active fragment or polypeptide is one having an in vivo or in vitro activity which is characteristic of the S. saprophyticus polypeptides of the invention, or of other naturally occurring S. saprophyticus polypeptides, e.g., one or more of the biological activities described herein. Especially preferred are fragments which exist in vivo, e.g., fragments which arise from post transcriptional processing or which arise from translation of alternatively spliced RNA's. Fragments include those expressed in native or endogenous cells as well as those made in expression systems, e.g., in E. coli cells. Because peptides such as S. saprophyticus polypeptides often exhibit a range of physiological properties and because such properties may be attributable to different portions of the molecule, a useful S. saprophyticus fragment or S. saprophyticus analog is one which exhibits a biological activity in any biological assay for S. saprophyticus activity. Most preferably the fragment or analog possesses 10%, preferably 40%, more preferably 60%, 70%, 80% or 90% or greater of the activity of S. saprophyticus, in any in vivo or in vitro assay.

As used herein, the term “fragment”, as applied to an S. saprophyticus polypeptide, will ordinarily be at least about 10, 15, 20, 25, 30, 35, 40, 45, 50, 55, 60, 65, 70, 75, 80, 85, 90,95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, or more amino acids. Fragments of S. saprophyticus polypeptides can be generated by methods known to those skilled in the art. The ability of a candidate fragment to exhibit a biological activity of S. saprophyticus polypeptide can be assessed by methods known to those skilled in the art as described herein. Also included are S. saprophyticus polypeptides containing residues that are not required for biological activity of the peptide or that result from alternative mRNA splicing or alternative protein processing events.

An “immunogenic component” as used herein is a moiety, such as an S. saprophyticus polypeptide, analog or fragment thereof, that is capable of eliciting a humoral and/or cellular immune response in a host animal alone or in combination with an adjuvant or a live vaccine carrier strain.

An “antigenic component” as used herein is a moiety, such as an S. saprophyticus polypeptide, analog or fragment thereof, that is capable of binding to a specific antibody with sufficiently high affinity to form a detectable antigen-antibody complex.

As used herein, the term “transgene” means a nucleic acid (encoding, e.g., one or more polypeptides), which is partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the cell's genome in such a way as to alter the genome of the cell into which it is inserted (e.g., it is inserted at a location which differs from that of the natural gene or its insertion results in a knockout). A transgene can include one or more transcriptional regulatory sequences and any other nucleic acid, such as introns, that may be necessary for optimal expression of the selected nucleic acid, all operably linked to the selected nucleic acid, and may include an enhancer sequence.

As used herein, the term “transgenic cell” refers to a cell containing a transgene.

As used herein, a “transgenic animal” is any animal in which one or more, and preferably essentially all, of the cells of the animal includes a transgene. The transgene can be introduced into the cell, directly or indirectly by introduction into a precursor of the cell, by way of deliberate genetic manipulation, such as by a process of transformation of competent cells or by microinjection or by infection with a recombinant virus. This molecule may be integrated within a chromosome, or it may be extrachromosomally replicating DNA.

The term “antibody” as used herein is intended to include fragments thereof which are specifically reactive with S. saprophyticus polypeptides.

As used herein, the term “cell-specific promoter” means a DNA sequence that serves as a promoter, i.e., regulates expression of a selected DNA sequence operably linked to the promoter, and which effects expression of the selected DNA sequence in specific cells of a tissue. The term also covers so-called “leaky” promoters, which regulate expression of a selected DNA primarily under certain conditions. The term also covers promoters which regulate expression of a selected DNA, during specific phases of infection.

Misexpression, as used herein, refers to a non-wild type pattern of gene expression. It includes: expression at non-wild type levels, i.e., over or under expression; a pattern of expression that differs from wild type in terms of the time or stage at which the gene is expressed, e.g., increased or decreased expression (as compared with wild type) at a predetermined infectious period or stage; a pattern of expression that differs from wild type in terms of decreased expression (as compared with wild type) in a predetermined host cell type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene, e.g., a pattern of increased or decreased expression (as compared with wild type) in the presence of an increase or decrease in the strength of the stimulus.

As used herein, “host cells” and other such terms denoting microorganisms or higher eukaryotic cell lines cultured as unicellular entities refers to cells which can become or have been used as recipients for a recombinant vector or other transfer DNA, and include the progeny of the original cell which has been introduced. It is understood by individuals skilled in the art that the progeny of a single parental cell may not necessarily be completely identical in genomic or total DNA compliment to the original parent, due to accident or deliberate mutation.

As used herein, the term “control sequence” refers to a nucleic acid having a base sequence which is recognized by the host organism to effect the expression of encoded sequences to which they are ligated. The nature of such control sequences differs depending upon the host organism; in prokaryotes, such control sequences generally include a promoter, ribosomal binding site, terminators, and in some cases operators; in eukaryotes, generally such control sequences include promoters, terminators and in some instances, enhancers. The term control sequence is intended to include at a minimum, all components whose presence is necessary for expression, and may also include additional components whose presence is advantageous, for example, leader sequences.

As used herein, the term “operably linked” refers to sequences joined or ligated to function in their intended manner. For example, a control sequence is operably linked to coding sequence by ligation in such a way that expression of the coding sequence is achieved under conditions compatible with the control sequence and host cell.

The metabolism of a substance, as used herein, means any aspect of the, expression, function, action, or regulation of the substance. The metabolism of a substance includes modifications, e.g., covalent or non-covalent modifications of the substance. The metabolism of a substance includes modifications, e.g., covalent or non-covalent modification, the substance induces in other substances. The metabolism of a substance also includes changes in the distribution of the substance. The metabolism of a substance includes changes the substance induces in the distribution of other substances.

A “sample” as used herein refers to a biological sample, such as, for example, tissue or fluid isolated from an individual (including without limitation plasma, serum, cerebrospinal fluid, lymph, tears, saliva, cells, urine, and tissue sections) or from in vitro cell culture constituents, as well as samples from the environment.

As used herein, the term “treatment” or “treating” is defined as the application or administration of a therapeutic agent to a patient, or application or administration of a therapeutic agent to an isolated tissue or cell line from a patient, who has a disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease. In one embodiment, the disease or disorder is treated by the inhibition or elimination of infection, e.g., a S. saprophyticus infection by an agent with inhibits S. saprophyticus nucleic acid expression or polypeptide activity, inhibits S. saprophyticus growth, or kills S. saprophyticus bacterial cells, e.g., in a subject.

The practice of the invention will employ, unless otherwise indicated, conventional techniques of chemistry, molecular biology, microbiology, recombinant DNA, and immunology, which are within the skill of the art. Such techniques are explained fully in the literature. See e.g., Sambrook, Fritsch, and Maniatis, Molecular Cloning; Laboratory Manual 2nd ed. (1989); DNA Cloning, Volumes I and II (D. N Glover ed. 1985); Oligonucleotide Synthesis (M. J. Gait ed, 1984); Nucleic Acid Hybridization (B. D. Hames & S. J. Higgins eds. 1984); the series, Methods in Enzymoloqy (Academic Press, Inc.), particularly Vol. 154 and Vol. 155 (Wu and Grossman, eds.) and PCR-A Practical Approach (McPherson, Quirke, and Taylor, eds., 1991).

Aspects of the invention are further explicated below.

I. Isolated Nucleic Acid Molecules

One aspect of the invention pertains to isolated nucleic acid molecules that encode polypeptides of the invention or biologically active portions thereof, as well as nucleic acid fragments sufficient for use as hybridization probes or primers for the identification or amplification of the nucleic acids of the invention or related nucleic acids. As used herein, the term “nucleic acid molecule” is intended to include DNA molecules (e.g., cDNA or genomic DNA) and RNA molecules (e.g., mRNA) and analogs of the DNA or RNA generated using nucleotide analogs. This term also encompasses untranslated sequence located at both the 3′ and 5′ ends of the coding region of the gene: at least about 100 nucleotides of sequence upstream from the 5′ end of the coding region and at least about 20 nucleotides of sequence downstream from the 3′ end of the coding region of the gene. The nucleic acid molecule can be single-stranded or double-stranded, but preferably is double-stranded DNA. An “isolated” nucleic acid molecule is one which is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived (e.g, an S. saprophyticus cell). Moreover, an “isolated” nucleic acid molecule, such as a DNA molecule, can be substantially free of other cellular material, or culture medium when produced by recombinant techniques, or chemical precursors or other chemicals when chemically synthesized.

A nucleic acid molecule of the present invention, e.g., a nucleic acid molecule having a nucleotide sequence as described in FIG. 1, or a portion thereof, can be isolated using standard molecular biology techniques and the sequence information provided herein. For example, an S. saprophyticus DNA can be isolated from an S. saprophyticus library using all or portion of one of the sequences of FIG. 1 as a hybridization probe and standard hybridization techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989). Moreover, a nucleic acid molecule encompassing all or a portion of one of the sequences of FIG. 1 can be isolated by the polymerase chain reaction using oligonucleotide primers designed based upon this sequence (e.g., a nucleic acid molecule encompassing all or a portion of one of the sequences of FIG. 1 can be isolated by the polymerase chain reaction using oligonucleotide primers designed based upon this same sequence of FIG. 1). For example, mRNA can be isolated from normal bacterial cells (e.g., by the guanidinium-thiocyanate extraction procedure of Chirgwin et al. (1979) Biochemistry 18: 5294-5299) and DNA can be prepared using reverse transcriptase (e.g., Moloney MLV reverse transcriptase, available from Gibco/BRL, Bethesda, MD; or AMV reverse transcriptase, available from Seikagaku America, Inc., St. Petersburg, Fla.). Synthetic oligonucleotide primers for polymerase chain reaction amplification can be designed based upon one of the nucleotide sequences shown in FIG. 1. A nucleic acid of the invention can be amplified using cDNA or, alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to a nucleotide sequence can be prepared by standard synthetic techniques, e.g., using an automated DNA synthesizer.

In a preferred embodiment, an isolated nucleic acid molecule of the invention comprises one of the nucleotide sequences shown in FIG. 1. The sequences of FIG. 1 correspond to the S. saphrophyticus DNAs of the invention. This DNA comprises sequences encoding proteins (i.e., the “coding region”, indicated in FIG. 1 for each encoded polypeptide), as well as 5′ untranslated sequences and 3′ untranslated sequences. Alternatively, the nucleic acid molecule can comprise only the coding region of any of the sequences in FIG. 1.

In another preferred embodiment, an isolated nucleic acid molecule of the invention comprises a nucleic acid molecule which is a complement of one of the nucleotide sequences shown in FIG. 1, or a portion thereof. A nucleic acid molecule which is complementary to one of the nucleotide sequences shown in FIG. 1 is one which is sufficiently complementary to one of the nucleotide sequences shown in FIG. 1 such that it can hybridize to one of the nucleotide sequences shown in FIG. 1, thereby forming a stable duplex.

In still another preferred embodiment, an isolated nucleic acid molecule of the invention comprises a nucleotide sequence which is at least about 50%, 51%, 52%, 53%, 54%, 55%, 56%, 57%, 58%, 59%, or 60%, preferably at least about 61%, 62%, 63%, 64%, 65%, 66%, 67%, 68%, 69%, or 70%%, more preferably at least about 71%, 72%, 73%, 74%, 75%, 76%, 77%, 78%, 79%, or 80%, 81%, 82%, 83%, 84%, 85%, 86%, 87%, 88%, 89%, or 90%, or 91%, 92%, 93%, 94%, and even more preferably at least about 95%, 96%, 97%, 98%, 99% or more homologous to a nucleotide sequence shown in FIG. 1, or a portion thereof.

As used herein, ranges and identity values intermediate to the above-recited ranges, (e.g., 70-90% identical or 80-95% identical) are also intended to be encompassed by the present invention. For example, ranges of identity values using a combination of any of the above values recited as upper and/or lower limits are intended to be included. In an additional preferred embodiment, an isolated nucleic acid molecule of the invention comprises a nucleotide sequence which hybridizes, e.g., hybridizes under stringent conditions, to one of the nucleotide sequences shown in FIG. 1, or a fragment thereof.

Moreover, the nucleic acid molecule of the invention can comprise only a portion of the coding region or the non-coding region of one of the sequences in FIG. 1, for example a fragment which can be used as a probe or primer or a fragment encoding a biologically active portion of a protein of the invention.

Nucleic acids isolated or synthesized in accordance with features of the present invention are useful, by way of example, without limitation, as probes, primers, capture ligands, antisense genes and for developing expression systems for the synthesis of proteins and peptides corresponding to such sequences. As probes, primers, capture ligands and antisense agents, the nucleic acid normally consists of all or part of the nucleic acids of the invention contained in FIG. 1. Probes and primers may be used, for example, identifying and/or cloning homologues in other cell types and organisms, as well as homologues from other Staphylococcus or related species.

A probe/primer typically comprises substantially purified oligonucleotide. The oligonucleotide typically comprises a region of nucleotide sequence that hybridizes under stringent conditions to at least about 12, 15, 16, 17, 18, 19, 20, 21, 22, 23, 24, preferably about 25, 26, 27, 28, 29, 30, 31, 32, 33, 34, 35, 36, 37, 38, 39, more preferably about 40, 50 or 75 consecutive nucleotides of a sense strand of one of the sequences set forth in FIG. 1, an anti-sense sequence of one of the sequences set forth in FIG. 1, or naturally occurring mutants thereof. Primers based on a nucleotide sequence of FIG. 1 can be used in PCR reactions to clone homologues. Probes based on the nucleotide sequences can be used to detect transcripts or genomic sequences encoding the same or homologous proteins. In preferred embodiments, the probe further comprises a label group attached thereto, e.g. the label group can be a radioisotope, a fluorescent compound, an enzyme, or an enzyme co-factor. Such probes can be used as a part of a diagnostic test kit for identifying cells which misexpress a protein, such as by measuring a level of a Staphylococcus polypeptide encoding nucleic acid in a sample of cells from a subject.

In one embodiment, the nucleic acid molecule of the invention encodes a protein or portion thereof which includes an amino acid sequence which is sufficiently homologous to an amino acid sequence encoded by the nucleic acid sequences of FIG. 1 such that the protein or portion thereof maintains activity. As used herein, the language “sufficiently homologous” refers to proteins or portions thereof which have amino acid sequences which include a minimum number of identical or equivalent amino acid residues to an amino acid sequence encoded by the nucleic acid sequences of FIG. 1 such that the protein or portion thereof is able to maintain an S. saprophyticus polypeptide activity. Examples of such activities are also described herein. Thus, “S. saprophyticus polypeptide activity” includes activities as set forth in FIG. 1 or which has one, two and preferably more of the following properties: ((1) any activity related to or associated with the initiation or progression of infection or disease, e.g., in a subject; (2) if when expressed in the course of an S. saprophyticus infection, it can promote, or mediate the attachment of S. saprophyticus to a cell; (3) it has an enzymatic activity, structural or regulatory function characteristic of an S. saprophyticus protein; (4) the gene which encodes it can rescue a lethal mutation in an S. saprophyticus gene; (5) or it is immunogenic in a subject. A polypeptide has biological activity if it is an antagonist, agonist, or super-agonist of a polypeptide having one of the above-listed properties.

In another embodiment, the protein is at least about 50%, 55, 60%, 65%, 70%, 75%, 80%, 85%, 90%, 91%, 92%, 93%, 94%, 95%, 96%, 97%, 98%, 99% or more homologous to an entire amino acid sequence encoded by the nucleic acid sequences of FIG. 1.

Portions of proteins encoded by the nucleic acid molecules of the invention are preferably biologically active portions of one of the proteins. As used herein, the term “biologically active portion of a protein” is intended to include a portion, e.g., a domain/motif, of a protein that retains an S. saprophyticus biological activity or has an activity as set forth in FIG. 1. To determine whether a protein or a biologically active portion thereof can retain an S. saprophyticus biological an assay for the specific biological activity may be performed. Additional nucleic acid fragments encoding biologically active portions of a protein can be prepared by isolating a portion of one of the sequences in FIG. 1, expressing the encoded portion of the protein or peptide (e.g., by recombinant expression in vitro) and assessing the activity of the encoded portion of the protein or peptide.

The invention further encompasses nucleic acid molecules that differ from one of the nucleotide sequences shown in FIG. 1 (and fragments thereof) due to degeneracy of the genetic code and thus encode the same protein as that encoded by the nucleotide sequences shown in FIG. 1. In another embodiment, an isolated nucleic acid molecule of the invention has a nucleotide sequence encoding a protein having an amino acid sequence shown in FIG. 1. In a still further embodiment, the nucleic acid molecule of the invention encodes a full length S. saprophyticus protein which is substantially homologous to an amino acid sequence encoded by the nucleic acid sequences of FIG. 1 (encoded by an open reading frame of a nucleotide sequence shown in FIG. 1).

It will be understood by one of ordinary skill in the art that in one embodiment the sequences of the invention are not meant to include the sequences of the prior art. In one embodiment, the invention includes nucleotide and amino acid sequences having a percent identity to a nucleotide or amino acid sequence of the invention which is greater than that of a sequence of the prior art. One of ordinary skill in the art would be able to calculate the lower threshold of percent identity for any given sequence of the invention by examining the GAP-calculated percent identity scores set forth in FIG. 1 for a hit for the given sequence, and by subtracting the highest GAP-calculated percent identity from 100 percent. One of ordinary skill in the art will also appreciate that nucleic acid and amino acid sequences having percent identities greater than the lower threshold so calculated (e.g., at least 50%, 51%, 52%, 53%, 54%, 55%, 56%, 57%, 58%, 59%, or 60%, preferably at least about 61%, 62%, 63%, 64%, 65%, 66%, 67%, 68%, 69%, or 70%, more preferably at least about 71%, 72%, 73%, 74%, 75%, 76%, 77%, 78%, 79%, or 80%, 81%, 82%, 83%, 84%, 85%, 86%, 87%, 88%, 89%, or 90%, or 91%, 92%, 93%, 94%, and even more preferably at least about 95%, 96%, 97%, 98%, 99% or more identical) are also encompassed by the invention.

In addition to the S. saprophyticus nucleotide sequences shown in FIG. 1, it will be appreciated by one of ordinary skill in the art that DNA sequence polymorphisms that lead to changes in the amino acid sequences of proteins may exist within a population (e.g., the S. saprophyticus population). Such genetic polymorphism in the gene may exist among a strain within a population due to natural variation. As used herein, the terms “gene” and “recombinant gene” refer to nucleic acid molecules comprising an open reading frame encoding a protein, preferably an S. saprophyticus protein. Such natural variations can typically result in 1-10% variance in the nucleotide sequence of the gene. Any and all such nucleotide variations and resulting amino acid polymorphisms in that are the result of natural variation and that do not alter the functional activity of proteins are intended to be within the scope of the invention.

Nucleic acid molecules corresponding to natural variants and non-S. saprophyticus homologues of the S. saprophyticus DNA of the invention can be isolated based on their homology to the S. saprophyticus nucleic acid disclosed herein using the S. saprophyticus DNA, or a fragment thereof, as a hybridization probe according to standard hybridization techniques under stringent hybridization conditions. Accordingly, in another embodiment, an isolated nucleic acid molecule of the invention is at least 15, 20, 25, 30 ,35, 40 ,45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250 or more nucleotides in length and hybridizes under stringent conditions to the nucleic acid molecule comprising a nucleotide sequence of FIG. 1. In other embodiments, the nucleic acid is at least 30, 40, 50, 60, 70, 80, 90, 100, 110, 120, 130, 140, 150, 160, 170, 180, 190, 200, 210, 220, 230, 240, 250 or more nucleotides in length.

As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4 and 6. Additional stringent conditions can be found in Molecular Cloning: A Laboratory Manual, Sambrook et al., Cold Spring Harbor Press, Cold Spring Harbor, N.Y. (1989), chapters 7, 9 and 11. A preferred, non-limiting example of stringent hybridization conditions includes hybridization in 4× sodium chloride/sodium citrate (SSC), at about 65-70° C. (or hybridization in 4× SSC plus 50% formamide at about 42-50° C.) followed by one or more washes in 1× SSC, at about 65-70° C. A preferred, non-limiting example of highly stringent hybridization conditions includes hybridization in 1× SSC, at about 65-70° C. (or hybridization in 1× SSC plus 50% formamide at about 42-50° C.) followed by one or more washes in 0.3× SSC, at about 65-70° C. A preferred, non-limiting example of reduced stringency hybridization conditions includes hybridization in 4× SSC, at about 50-60° C. (or alternatively hybridization in 6× SSC plus 50% formamide at about 40-45° C.) followed by one or more washes in 2× SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25mM EDTA, pH 7.4) can be substituted for SSC (1×SSC is 0.15M NaCl and 15mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(°C.)=2(# of A+T bases)+4(# of G+C bases). For hybrids between 18 and 49 base pairs in length, Tₘ(°C.)=81.5 +16.6(log₁₀[Na⁺])+0.41(%G+C)−(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C., see e.g., Church and Gilbert (1984) Proc. Natl. Acad. Sci. USA 81:1991-1995, (or alternatively 0.2× SSC, 1% SDS).

In addition to naturally-occurring variants of the sequence that may exist in the population, one of ordinary skill in the art will further appreciate that changes can be introduced by mutation into a nucleotide sequence of FIG. 1, thereby leading to changes in the amino acid sequence of the encoded protein, without altering the functional ability of the protein. For example, nucleotide substitutions leading to amino acid substitutions at “non-essential” amino acid residues can be made in a sequence of FIG. 1. A “non-essential” amino acid residue is a residue that can be altered from the wild-type sequence of one of the proteins (FIG. 1) without altering the activity of said protein, whereas an “essential” amino acid residue is required for protein activity. Other amino acid residues, however, (e.g., those that are not conserved or only semi-conserved in the domain having activity) may not be essential for activity and thus are likely to be amenable to alteration without altering activity.

Accordingly, another aspect of the invention pertains to nucleic acid molecules encoding proteins that contain changes in amino acid residues that are not essential for activity. Such proteins differ in amino acid sequence from a sequence contained in FIG. 1 yet retain at least one of the activities described herein. In one embodiment, the isolated nucleic acid molecule comprises a nucleotide sequence encoding a protein of the invention, wherein the protein comprises an amino acid sequence at least about 50% homologous to an amino acid sequence encoded by the nucleic acid sequences of FIG. 1 and is capable of retaining an S. saprophyticus biological activity, or has one or more activities set forth in FIG. 1. Preferably, the protein encoded by the nucleic acid molecule is at least about 50,%, 51 %, 52%, 53%, 54%, 55%, 56%, 57%, 58%, 59%, 60% homologous to one of the sequences in FIG. 1, more preferably at least about 60%, 61%, 62%, 63%, 64%, 65%, 66%, 67%, 68%, 69%, 70% homologous to one of the sequences in FIG. 1, even more preferably at least about 70%, 75%, 80%, 85%, 90%, 95% homologous to one of the sequences in FIG. 1, and most preferably at least about 96%, 97%, 98%, or 99% homologous to one of the sequences in FIG. 1.

In another embodiment, the present invention includes S. saprophyticus polypeptides which have been mutated, e.g., by a change in the nucleotide sequence encoding the polypeptide, which leads to change in the activity of the polypeptide or a modulation of drug resistance by the bacteria, e.g., wherein the change renders the bacteria partially or completely resistant to treatment with a particular compound.

To determine the percent homology of two amino acid sequences (e.g., one of the sequences of FIG. 1 and a mutant form thereof) or of two nucleic acids, the sequences are aligned for optimal comparison purposes (e.g., gaps can be introduced in the sequence of one protein or nucleic acid for optimal alignment with the other protein or nucleic acid). The amino acid residues or nucleotides at corresponding amino acid positions or nucleotide positions are then compared. When a position in one sequence (e.g., one of the sequences of FIG. 1) is occupied by the same amino acid residue or nucleotide as the corresponding position in the other sequence (e.g., a mutant form of the sequence selected from FIG. 1), then the molecules are homologous at that position (i.e., as used herein amino acid or nucleic acid “homology” is equivalent to amino acid or nucleic acid “identity”). The percent homology between the two sequences is a function of the number of identical positions shared by the sequences (i.e., % homology=# of identical positions/total # of positions×100).

An isolated nucleic acid molecule encoding a protein homologous to a protein sequence of FIG. 1 can be created by introducing one or more nucleotide substitutions, additions or deletions into a nucleotide sequence of FIG. 1 such that one or more amino acid substitutions, additions or deletions are introduced into the encoded protein. Mutations can be introduced into one of the sequences of FIG. 1 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a protein is preferably replaced with another amino acid residue from the same side chain family. Alternatively, in another embodiment, mutations can be introduced randomly along all or part of a coding sequence, such as by saturation mutagenesis, and the resultant mutants can be screened for an activity described herein to identify mutants that retain activity. Following mutagenesis of one of the sequences of FIG. 1, the encoded protein can be expressed recombinantly and the activity of the protein can be determined using, for example, assays described herein.

In addition to the nucleic acid molecules encoding proteins described above, another aspect of the invention pertains to isolated nucleic acid molecules which are antisense thereto. Nucleic acid or nucleic acid-hybridizing derivatives isolated or synthesized in accordance with the sequences described herein have utility as antisense agents to prevent the expression of S. saprophyticus genes. These sequences also have utility as antisense agents to prevent expression of genes of other Staphylococcus species.

An “antisense” nucleic acid comprises a nucleotide sequence which is complementary to a “sense” nucleic acid encoding a protein, e.g., complementary to the coding strand of a double-stranded DNA molecule or complementary to an mRNA sequence. Accordingly, an antisense nucleic acid can hydrogen bond to a sense nucleic acid. The antisense nucleic acid can be complementary to an entire coding strand, or to only a portion thereof. In one embodiment, an antisense nucleic acid molecule is antisense to a coding region of the coding strand of a nucleotide sequence encoding a protein. In another embodiment, the antisense nucleic acid molecule is antisense to a “noncoding region” of the coding strand of a nucleotide sequence encoding. The term “noncoding region” refers to 5′ and 3′ sequences which flank the coding region that are not translated into amino acids (i.e., also referred to as 5′ and 3′ untranslated regions).

Given the coding strand sequences encoding polypeptides disclosed herein (e.g., the sequences set forth in FIG. 1), antisense nucleic acids of the invention can be designed according to the rules of Watson and Crick base pairing. The antisense nucleic acid molecule can be complementary to the entire coding region of mRNA, but more preferably is an oligonucleotide which is antisense to only a portion of the coding or noncoding region of mRNA. For example, the antisense oligonucleotide can be complementary to the region surrounding the translation start site of mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a cell or generated in situ such that they hybridize with or bind to cellular mRNA and/or genomic DNA encoding a protein of the invention to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. The antisense molecule can be modified such that it specifically binds to a receptor or an antigen expressed on a selected cell surface, e.g., by linking the antisense nucleic acid molecule to a peptide or an antibody which binds to a cell surface receptor or antigen. The antisense nucleic acid molecule can also be delivered to cells using the vectors described herein. To achieve sufficient intracellular concentrations of the antisense molecules, vector constructs in which the antisense nucleic acid molecule is placed under the control of a strong prokaryotic, viral, or eukaryotic promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave mRNA transcripts to thereby inhibit translation of mRNA. A ribozyme having specificity for an -encoding nucleic acid can be designed based upon the nucleotide sequence of aDNA disclosed herein. For example, a derivative of a Tetrahymena L-19 IVS RNA can be constructed in which the nucleotide sequence of the active site is complementary to the nucleotide sequence to be cleaved in an -encoding mRNA. See, e.g., Cech et al. U.S. Pat. No. 4,987,071 and Cech et al. U.S. Pat. No. 5,116,742. Alternatively, mRNA can be used to select a catalytic RNA having a specific ribonuclease activity from a pool of RNA molecules. See, e.g., Bartel, D. and Szostak, J. W. (1993) Science 261:1411-1418.

Alternatively, gene expression can be inhibited by targeting nucleotide sequences complementary to the regulatory region of a nucleotide sequence (e.g., an promoter and/or enhancers) to form triple helical structures that prevent transcription of an gene in target cells. See generally, Helene, C. (1991) Anticancer Drug Des. 6(6):569-84; Helene, C. et al. (1992) Ann. N.Y Acad. Sci. 660:27-36; and Maher, L. J. (1992) Bioassays 14(12):807-15.

II. Recombinant Expression Vectors and Host Cells

Another aspect of the invention pertains to vectors, preferably expression vectors, containing a nucleic acid encoding a protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., bacterial suicide vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as bacteriophages or viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, repressor binding sites, activator binding sites, enhancers and other expression control elements (e.g., terminators, polyadenylation signals, or other elements of mRNA secondary structure). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells. Preferred regulatory sequences are, for example, promoters such as cos-, tac-, trp-, tet-, trp-tet-, lpp-, lac-, lpp-lac-, lacI^{q}-, T7-, T5-, T3-, gal-, trc-, ara-, SP6-, arny, SPO2, λ-P_{R}- or λ P_{L}, which are used preferably in bacteria. Additional regulatory sequences are, for example, promoters from yeasts and fungi, such as ADC1, MFα, AC, P-60, CYC1, GAPDH, TEF, rp28, ADH, promoters from plants such as CaMV/35S, SSU, OCS, lib4, usp, STLS1, B33, nos or ubiquitin- or phaseolin-promoters. It is also possible to use artificial promoters. It will be appreciated by one of ordinary skill in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., proteins, mutant forms of proteins, fusion proteins, etc.).

The recombinant expression vectors of the invention can be designed for expression of proteins in prokaryotic or eukaryotic cells. For example, genes can be expressed in bacterial cells such as S. saprophyticus or E. coli, insect cells (using baculovirus expression vectors), yeast and other fungal cells (see Romanos, M. A. et al. (1992) “Foreign gene expression in yeast: a review”, Yeast 8: 423-488; van den Hondel, C. A. M. J. J. et al. (1991) “Heterologous gene expression in filamentous fungi” in: More Gene Manipulations in Fungi, J. W. Bennet & L. L. Lasure, eds., p. 396-428: Academic Press: San Diego; and van den Hondel, C. A. M. J. J. & Punt, P. J. (1991) “Gene transfer systems and vector development for filamentous fungi, in: Applied Molecular Genetics of Fungi, Peberdy, J. F. et al., eds., p. 1-28, Cambridge University Press: Cambridge), algae and multicellular plant cells (see Schmidt, R. and Willmitzer, L. (1988) High efficiency Agrobacterium tumefaciens -mediated transformation of Arabidopsis thaliana leaf and cotyledon explants” Plant Cell Rep.: 583-586), or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out with vectors containing constitutive or inducible promoters directing the expression of either fusion or non-fusion proteins. Fusion vectors add a number of amino acids to a protein encoded therein, usually to the amino terminus of the recombinant protein but also to the C-terminus or fused within suitable regions in the proteins. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase.

Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. In one embodiment, the coding sequence of the protein is cloned into a pGEX expression vector to create a vector encoding a fusion protein comprising, from the N-terminus to the C-terminus, GST-thrombin cleavage site-X protein. The fusion protein can be purified by affinity chromatography using glutathione-agarose resin. Recombinant protein unfused to GST can be recovered by cleavage of the fusion protein with thrombin.

Examples of suitable inducible non-fusion E. coli expression vectors include pTrc (Amann et al., (1988) Gene 69:301-315) pLG338, pACYC184, pBR322, pUC18, pUC19, pKC30, pRep4, pHS1, pHS2, pPLc236, pMBL24, pLG200, pUR290, pIN-III113-B1, λgt11, pBdCl, and pET11d (Studier et al., Gene Expression Technology: Methods in Enzymology 185, Academic Press, San Diego, Calif. (1990) 60-89; and Pouwels et al., eds. (1985) Cloning Vectors. Elsevier: New York IBSN 0 444 904018). Target gene expression from the pTrc vector relies on host RNA polymerase transcription from a hybrid trp-lac fusion promoter. Target gene expression from the pET11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident λ prophage harboring a T7 gn1 gene under the transcriptional control of the lacUV 5 promoter. For transformation of other varieties of bacteria, appropriate vectors may be selected.

One strategy to maximize recombinant protein expression is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in the bacterium chosen for expression, such as S. saprophyticus orE. coli (Wada et al. (1992) Nucleic Acids Res. 20:2111-2118). Such alteration of nucleic acid sequences of the invention can be carried out by standard DNA synthesis techniques.

In another embodiment, the protein expression vector is a yeast expression vector. Examples of vectors for expression in yeast S. cerevisiae include pYepSec 1 (Baldari, et al., (1987) Embo J. 6:229-234), 2 μ, pAG-1, Yep6, Yep13, pEMBLYe23, pMFa (Kurjan and Herskowitz, (1982) Cell 30:933-943), pJRY88 (Schultz et al., (1987) Gene 54:113-123), and pYES2 (Invitrogen Corporation, San Diego, Calif.). Vectors and methods for the construction of vectors appropriate for use in other fungi, such as the filamentous fungi, include those detailed in: van den Hondel, C. A. M. J. J. & Punt, P.J. (1991) “Gene transfer systems and vector development for filamentous fungi, in: Applied Molecular Genetics of Fungi, J. F. Peberdy, et al., eds., p. 1-28, Cambridge University Press: Cambridge, and Pouwels et al., eds. (1985) Cloning Vectors. Elsevier: New York (IBSN 0 444 904018).

Alternatively, the proteins of the invention can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In another embodiment, the proteins of the invention may be expressed in unicellular plant cells (such as algae) or in plant cells from higher plants (e.g., the spermatophytes, such as crop plants). Examples of plant expression vectors include those detailed in: Becker, D., Kemper, E., Schell, J. and Masterson, R. (1992) “New plant binary vectors with selectable markers located proximal to the left border”, Plant Mol. Biol. 20: 1195-1197; and Bevan, M. W. (1984) “Binary Agrobacterium vectors for plant transformation”, Nucl. Acid. Res. 12: 8711-8721, and include pLGV23, pGHlac+, pBIN19, pAK2004, and pDH51 (Pouwels et al., eds. (1985) Cloning Vectors. Elsevier: New York IBSN 0 444 904018).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific, e.g., bacterial cell specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a protein of the invention can be expressed in bacterial cells such as Staphylococcus or Escherichia cells, e.g., S. saprophyticus or E. coli cells, insect cells, yeast or mammalian cells (such as Chinese hamster ovary cells (CHO) or COS cells). Other suitable host cells are known to those of ordinary skill in the art.

Vector DNA can be introduced into prokaryotic or eukaryotic cells via conventional transformation or transfection techniques. As used herein, the terms “transformation” and “transfection”, “conjugation” and “transduction” are intended to refer to a variety of art-recognized techniques for introducing foreign nucleic acid (e.g., linear DNA or RNA (e.g., a linearized vector or a gene construct alone without a vector) or nucleic acid in the form of a vector (e.g., a plasmid, phage, phasmid, phagemid, transposon or other DNA) into a host cell, including calcium phosphate or calcium chloride co-precipitation, DEAE-dextran-mediated transfection, lipofection, natural competence, chemical-mediated transfer, conjugation, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding a protein or can be introduced on a separate vector. Cells stably transfected with the introduced nucleic acid can be identified by drug selection (e.g., cells that have incorporated the selectable marker gene will survive, while the other cells die).

To create a homologous recombinant microorganism, a vector is prepared which contains at least a portion of a gene of the invention into which a deletion, addition or substitution has been introduced to thereby alter, e.g., functionally disrupting the gene. Preferably, this gene is an S. saprophyticus gene, but it can be a homologue from a related bacterium or even from a mammalian, yeast, or insect source. In a preferred embodiment, the vector is designed such that, upon homologous recombination, the endogenous gene is functionally disrupted (i.e., no longer encodes a functional protein; also referred to as a “knock out” vector). Alternatively, the vector can be designed such that, upon homologous recombination, the endogenous gene is mutated or otherwise altered but still encodes functional protein (e.g., the upstream regulatory region can be altered to thereby alter the expression of the endogenous protein). In the homologous recombination vector, the altered portion of the gene is flanked at its 5′ and 3′ ends by additional nucleic acid of the gene to allow for homologous recombination to occur between the exogenous gene carried by the vector and an endogenous gene in a microorganism. The additional flanking nucleic acid is of sufficient length for successful homologous recombination with the endogenous gene. Typically, several hundred basepairs of flanking DNA are included in the vector (see e.g., Thomas, K. R., and Capecchi, M. R. (1987) Cell 51: 503 for a description of homologous recombination vectors). The vector is introduced into a microorganism (e.g., by electroporation) and cells in which the introduced gene has homologously recombined with the endogenous gene are selected, using art-known techniques.

In another embodiment, recombinant microorganisms can be produced which contain selected systems which allow for regulated expression of the introduced gene. For example, inclusion of a gene of the invention on a vector placing it under control of the lac operon permits expression of the gene only in the presence of an inducer such as, for example, IPTG. Such regulatory systems are well known in the art.

In another embodiment, an endogenous gene in a host cell is disrupted (e.g., by homologous recombination or other genetic means known in the art) such that expression of its protein product does not occur. In another embodiment, an endogenous or introduced gene in a host cell has been altered by one or more point mutations, deletions, or inversions, but still encodes a functional protein. In still another embodiment, one or more of the regulatory regions (e.g., a promoter, repressor, or inducer) of an gene in a microorganism has been altered (e.g., by deletion, insertion, truncation, inversion, or point mutation) such that the expression of the gene is modulated. One of ordinary skill in the art will appreciate that host cells containing more than one of the described gene and protein modifications may be readily produced using the methods of the invention, and are meant to be included in the present invention.

A host cell of the invention, such as a prokaryotic or eukaryotic host cell in culture, can be used to produce (i.e., express) a protein. Accordingly, the invention further provides methods for producing proteins using the host cells of the invention. In one embodiment, the method comprises culturing the host cell of invention (into which a recombinant expression vector encoding a protein has been introduced, or into which genome has been introduced a gene encoding a wild-type or altered protein) in a suitable medium until protein is produced. In another embodiment, the method further comprises isolating proteins from the medium or the host cell.

III. Isolated Polypeptides

Another aspect of the invention pertains to isolated proteins, and biologically active portions thereof. An “isolated” or “purified” protein or biologically active portion thereof is substantially free of cellular material when produced by recombinant DNA techniques, or chemical precursors or other chemicals when chemically synthesized. The language “substantially free of cellular material” includes preparations of protein in which the protein is separated from cellular components of the cells in which it is naturally or recombinantly produced. In one embodiment, the language “substantially free of cellular material” includes preparations of protein having less than about 30% (by dry weight) of non-protein (also referred to herein as a “contaminating protein” ), more preferably less than about 20% of non-protein, still more preferably less than about 10% of non-protein, and most preferably less than about 5% non-protein. When the protein or biologically active portion thereof is recombinantly produced, it is also preferably substantially free of culture medium, i.e., culture medium represents less than about 20%, more preferably less than about 10%, and most preferably less than about 5% of the volume of the protein preparation. The language “substantially free of chemical precursors or other chemicals” includes preparations of protein in which the protein is separated from chemical precursors or other chemicals which are involved in the synthesis of the protein. In one embodiment, the language “substantially free of chemical precursors or other chemicals” includes preparations of protein having less than about 30% (by dry weight) of chemical precursors or non-chemicals, more preferably less than about 20% chemical precursors or non-chemicals, still more preferably less than about 10% chemical precursors or non-chemicals, and most preferably less than about 5% chemical precursors or non-chemicals. In preferred embodiments, isolated proteins or biologically active portions thereof lack contaminating proteins from the same organism from which the protein is derived. Typically, such proteins are produced by recombinant expression of, for example, a S. saprophyticus protein of the invention in a microorganism such as S. saprophyticus.

An isolated protein or a portion thereof of the invention can retain an S. saprophyticus biological activity or has one or more of the activities set forth in FIG. 1. In preferred embodiments, the protein or portion thereof comprises an amino acid sequence which is sufficiently homologous to an amino acid sequence encoded by the nucleic acid sequences of FIG. 1 such that the protein or portion thereof maintains the ability to retain an S. saprophyticus biological activity in an amino acid sequence or has one or more of the activities set forth in FIG. 1. The portion of the protein is preferably a biologically active portion as described herein. In another preferred embodiment, a protein of the invention has an amino acid sequence shown in FIG. 1. In yet another preferred embodiment, the protein has an amino acid sequence which is encoded by a nucleotide sequence which hybridizes, e.g., hybridizes under stringent conditions, to a nucleotide sequence of FIG. 1. In still another preferred embodiment, the protein has an amino acid sequence which is encoded by a nucleotide sequence that is at least about 50%, 51%, 52%, 53%, 54%, 55%, 56%, 57%, 58%, 59%, or 60%, preferably at least about 61%, 62%, 63%, 64%, 65%, 66%, 67%, 68%, 69%, or 70%, more preferably at least about 71%, 72%, 73%, 74%, 75%, 76%, 77%, 78%, 79%, or 80%, 81%, 82%, 83%, 84%, 85%, 86%, 87%, 88%, 89%, or 90%, or 91%, 92%, 93%, 94%, and even more preferably at least about 95%, 96%, 97%, 98%, 99% or more homologous to one of the nucleic acid sequences of FIG. 1, or a portion thereof. Ranges and identity values intermediate to the above-recited values, (e.g., 70-90% identical or 80-95% identical) are also intended to be encompassed by the present invention. For example, ranges of identity values using a combination of any of the above values recited as upper and/or lower limits are intended to be included. The preferred proteins of the present invention also preferably possess at least one of the activities described herein. For example, a preferred protein of the present invention includes an amino acid sequence encoded by a nucleotide sequence which hybridizes, e.g., hybridizes under stringent conditions, to a nucleotide sequence of FIG. 1, and which retains an S. saprophyticus biological activity or which has one or more of the activities set forth in FIG. 1.

In other embodiments, the protein is substantially homologous to an amino acid sequence encoded by the nucleic acid sequence of FIG. 1 and retains the functional activity of the protein of one of the sequences of FIG. 1 yet differs in amino acid sequence due to natural variation or mutagenesis, as described in detail above. Accordingly, in another embodiment, the protein is a protein which comprises an amino acid sequence which is at least about 50%, 51%, 52%, 53%, 54%, 55%, 56%, 57%, 58%, 59%, or 60%, preferably at least about 61%, 62%, 63%, 64%, 65%, 66%, 67%, 68%, 69%, or 70%, more preferably at least about 71%, 72%, 73%, 74%, 75%, 76%, 77%, 78%, 79%, or 80%, 81%, 82%, 83%, 84%, 85%, 86%, 87%, 88%, 89%, or 90%, or 91%, 92%, 93%, 94%, and even more preferably at least about 95%, 96%, 97%, 98%, 99% or more homologous to an entire amino acid sequence encoded by the nucleic acid sequence of FIG. 1 and which has at least one of the activities described herein. Ranges and identity values intermediate to the above-recited values, (e.g., 70-90% identical or 80-95% identical) are also intended to be encompassed by the present invention. For example, ranges of identity values using a combination of any of the above values recited as upper and/or lower limits are intended to be included. In another embodiment, the invention pertains to a full length S. saprophyticus protein which is substantially homologous to an entire amino acid sequence encoded by a nucleic acid sequence of FIG. 1.

Biologically active portions of a protein include peptides comprising amino acid sequences derived from the amino acid sequence of a protein, e.g., the amino acid sequence encoded by the nucleic acid sequence shown in FIG. 1 or the amino acid sequence of a protein homologous to a protein, which include fewer amino acids than a full length protein or the full length protein which is homologous to a protein, and exhibit at least one activity of a protein. Typically, biologically active portions (peptides, e.g., peptides which are, for example, 5, 10, 15, 20, 30, 35, 40, 50, 60, 70, 80, 90, 100, 120, 130, 140, 150, 160, 170, 180, 190, 200, 225, 250, 275, 300, 325, 350, 375, or more amino acids in length) comprise a domain or motif with at least one activity of a protein. Moreover, other biologically active portions, in which other regions of the protein are deleted, can be prepared by recombinant techniques and evaluated for one or more of the activities described herein. Preferably, the biologically active portions of a protein of the invention include one or more selected domains/motifs or portions thereof having biological activity.

Proteins of the invention are preferably produced by recombinant DNA techniques. For example, a nucleic acid molecule encoding the protein is cloned into an expression vector (as described above), the expression vector is introduced into a host cell (as described above) and the protein is expressed in the host cell. The protein can then be isolated from the cells by an appropriate purification scheme using standard protein purification techniques. Alternative to recombinant expression, a protein, polypeptide, or peptide can be synthesized chemically using standard peptide synthesis techniques. Moreover, native protein can be isolated from cells (e.g., bacterial cells), for example using an anti-antibody, which can be produced by standard techniques utilizing a protein or fragment thereof of this invention.

The invention also provides chimeric or fusion proteins. As used herein, an “chimeric protein” or “fusion protein” comprises a polypeptide operatively linked to a non-polypeptide. An “polypeptide” refers to a polypeptide having an amino acid sequence corresponding to, whereas a “non-polypeptide” refers to a polypeptide having an amino acid sequence corresponding to a protein which is not substantially homologous to the protein, e.g., a protein which is different from the protein and which is derived from the same or a different organism. Within the fusion protein, the term “operatively linked” is intended to indicate that the polypeptide and the non-polypeptide are fused in-frame to each other. The non-polypeptide can be fused to the N-terminus or C-terminus of the polypeptide. For example, in one embodiment the fusion protein is a GST-fusion protein in which the sequences are fused to the C-terminus of the GST sequences. Such fusion proteins can facilitate the purification of recombinant proteins. In another embodiment, the fusion protein is a protein containing a heterologous signal sequence at its N-terminus. In certain host cells (e.g., mammalian host cells), expression and/or secretion of a protein can be increased through use of a heterologous signal sequence.

Preferably, a chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). An -encoding nucleic acid can be cloned into such an expression vector such that the fusion moiety is linked in-frame to the protein.

Homologues of the protein can be generated by mutagenesis, e.g., discrete point mutation or truncation of the protein. As used herein, the term “homologue” refers to a variant form of the protein which acts as an agonist or antagonist of the activity of the protein. An agonist of the protein can retain substantially the same, or a subset, of the biological activities of the protein. An antagonist of the protein can inhibit one or more of the activities of the naturally occurring form of the protein, by, for example, competitively binding to a downstream or upstream member of the cascade or pathway, e.g., metabolic pathway, which includes the protein.

In an alternative embodiment, homologues of the protein can be identified by screening combinatorial libraries of mutants, e.g., truncation mutants, of the protein for protein agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential sequences is expressible as individual polypeptides, or alternatively, as a set of larger fusion proteins (e.g., for phage display) containing the set of sequences therein. There are a variety of methods which can be used to produce libraries of potential homologues from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of the protein coding can be used to generate a variegated population of fragments for screening and subsequent selection of homologues of a protein. In one embodiment, a library of coding sequence fragments can be generated by treating a double stranded PCR fragment of a coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of homologues. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify homologues (Arkin and Yourvan (1992) PNAS 89:7811-7815; Delgrave et al. (1993) Protein Engineering 6(3):327-331).

In another embodiment, cell based assays can be exploited to analyze a variegated library, using methods well known in the art.

IV. Uses and Methods of the Invention

The nucleic acid molecules, proteins, protein homologues, fusion proteins, primers, vectors, and host cells described herein can be used in a variety of methods including, for example, one or more of the following methods: identification of S. saprophyticus and related organisms, diagnosis of an S. saprophyticus-associated disease or disorder, e.g., a urinary tract infection in a subject; mapping of genomes of organisms related to S. saprophyticus; identification and localization of S. saprophyticus sequences of interest; evolutionary studies; determination of protein regions required for function; modulation of an S. saprophyticus protein activity. Furthermore, the nucleic acid and polypeptide sequences of the invention are be useful for the identification of anti-bacterial compounds, e.g., small molecule inhibitors. In addition, the nucleic acid and polypeptide sequences of the invention can be utilized to elucidate anti-bacterial mechanisms. Moreover, the nucleic acid and polypeptide molecules of the invention may be utilized for epidemiological studies. For example, to better understand the spread of disease, e.g., the global spread of disease.

The nucleic acid molecules of the invention have a variety of uses. First, they may be used to identify an organism as being S. saprophyticus or a close relative thereof. Also, they may be used to identify the presence of S. saprophyticus or a relative thereof in a mixed population of microorganisms. The invention provides the nucleic acid sequences of a number of S. saprophyticus genes; by probing the extracted genomic DNA of a culture of a unique or mixed population of microorganisms under stringent conditions with a probe spanning a region of an S. saprophyticus gene which is unique to this organism, one can ascertain whether this organism is present.

V. Diagnostics

In one embodiment, the invention provides a method of identifying the presence or activity of S. saprophyticus in a subject. This method includes detection of one or more of the nucleic acid or amino acid sequences of the invention (e.g., the sequences set forth in FIG. 1 or FIG. 1) in a subject, thereby detecting the presence or activity of S. saprophyticus in the subject.

An exemplary method for detecting the presence or absence of a polypeptide of the invention in a subject involves obtaining a biological sample from a test subject and contacting the biological sample with a compound or an agent capable of detecting S. saprophyticus polypeptide or nucleic acid (e.g., mRNA, or genomic DNA) that encodes S. saprophyticus polypeptide such that the presence of S. saprophyticus polypeptide or nucleic acid is detected in the biological sample. In another aspect, the present invention provides a method for detecting the presence of S. saprophyticus activity in a biological sample by contacting the biological sample with an agent capable of detecting an indicator of S. saprophyticus biological activity such that the presence of S. saprophyticus biological activity is detected in the biological sample. A preferred agent for detecting S. saprophyticus mRNA or genomic DNA is a labeled nucleic acid probe capable of hybridizing to S. saprophyticus mRNA or genomic DNA. The nucleic acid probe can be, for example, the S. saprophyticus nucleic acid set forth in FIG. 1, or a portion thereof, such as an oligonucleotide of at least 15, 30, 50, 100, 250 or 500 nucleotides in length and sufficient to specifically hybridize under stringent conditions to S. saprophyticus mRNA or genomic DNA. Other suitable probes for use in the diagnostic assays of the invention are described herein.

A preferred agent for detecting an S. saprophyticus polypeptide is an antibody capable of binding to S. saprophyticus polypeptide, preferably an antibody with a detectable label. Antibodies can be polyclonal, or more preferably, monoclonal. An intact antibody, or a fragment thereof (e.g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, e.g., urine, as well as tissues, cells and fluids present within a subject. That is, the detection method of the invention can be used to detect S. saprophyticus mRNA, polypeptide, or genomic DNA in a biological sample in vitro as well as in vivo. For example, in vitro techniques for detection of S. saprophyticus mRNA include Northern hybridizations and in situ hybridizations as well as PCR based assays, e.g., using the probes and primers identified herein. In vitro techniques for detection of S. saprophyticus polypeptide include enzyme linked immunosorbent assays (ELISAs), Western blots, immunoprecipitations and immunofluorescence. In vitro techniques for detection of S. saprophyticus genomic DNA include Southern hybridizations. Furthermore, in vivo techniques for detection of S. saprophyticus polypeptide include introducing into a subject a labeled anti-S. saprophyticus antibody. For example, the antibody can be labeled with a radioactive marker whose presence and location in a subject can be detected by standard imaging techniques.

The present invention also provides diagnostic assays for identifying the presence or absence of a genetic alteration characterized by at least one of (i) aberrant modification or mutation of a gene encoding an S. saprophyticus polypeptide; (ii) aberrant expression of a gene encoding an S. saprophyticus polypeptide; (iii) mis-regulation of a gene of the present invention; and (iii) aberrant post-translational modification of an S. saprophyticus polypeptide, wherein a wild-type form of the gene encodes a polypeptide with an S. saprophyticus biological activity. “Misexpression or aberrant expression”, as used herein, refers to a non-wild type pattern of gene expression, at the RNA or protein level. It includes, but is not limited to, expression at non-wild type levels (e.g., over or under expression); a pattern of expression that differs from wild type in terms of the time or stage at which the gene is expressed (e.g., increased or decreased expression (as compared with wild type) at a predetermined developmental period or stage); a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene (e.g., a pattern of increased or decreased expression (as compared with wild type) in the presence of an increase or decrease in the strength of the stimulus).

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a control subject, contacting the control sample with a compound or agent capable of detecting S. saprophyticus polypeptide, mRNA, or genomic DNA, such that the presence of S. saprophyticus polypeptide, mRNA or genomic DNA is detected in the biological sample, and comparing the presence of S. saprophyticus polypeptide, MRNA or genomic DNA in the control sample with the presence of S. saprophyticus polypeptide, mRNA or genomic DNA in the test sample.

The invention also encompasses kits for detecting the presence of S. saprophyticus in a biological sample. For example, the kit can comprise a labeled compound or agent capable of detecting S. saprophyticus polypeptide or mRNA in a biological sample; means for determining the amount of S. saprophyticus in the sample; and means for comparing the amount of S. saprophyticus in the sample with a standard. The compound or agent can be packaged in a suitable container. The kit can further comprise instructions for using the kit to detect S. saprophyticus polypeptide or nucleic acid.

The nucleic acid and protein molecules of the invention may also serve as markers for specific regions of the genome. This has utility not only in the mapping of the genome, but also for functional studies of S. saprophyticus proteins. For example, to identify the region of the genome to which a particular S. saprophyticus DNA-binding protein binds, the S. saprophyticus genome could be digested, and the fragments incubated with the DNA-binding protein. Those which bind the protein may be additionally probed with the nucleic acid molecules of the invention, preferably with readily detectable labels; binding of such a nucleic acid molecule to the genome fragment enables the localization of the fragment to the genome map of S. saprophyticus, and, when performed multiple times with different enzymes, facilitates a rapid determination of the nucleic acid sequence to which the protein binds. Further, the nucleic acid molecules of the invention may be sufficiently homologous to the sequences of related species such that these nucleic acid molecules may serve as markers for the construction of a genomic map in related bacteria, such as e.g., a Staphyloccocus bacteria.

The nucleic acid and polypeptide molecules of the invention are also useful for evolutionary and protein structural studies. The metabolic processes in which the molecules of the invention participate are utilized by a wide variety of prokaryotic and eukaryotic cells; by comparing the sequences of the nucleic acid and polypeptide molecules of the present invention to those encoding similar enzymes from other organisms, the evolutionary relatedness of the organisms can be assessed. Similarly, such a comparison permits an assessment of which regions of the sequence are conserved and which are not, which may aid in determining those regions of the protein which are essential for the functioning of the enzyme. This type of determination is of value for protein engineering studies and may give an indication of what the protein can tolerate in terms of mutagenesis without losing function.

Manipulation of the nucleic acid molecules of the invention may result in the production of proteins having functional differences from the wild-type proteins. These proteins may be improved in efficiency or activity, may be present in greater numbers in the cell than is usual, or may be decreased in efficiency or activity.

The invention also provides methods for screening molecules which modulate the activity of a S. saprophyticus protein, either by interacting with the protein itself or a substrate or binding partner of the S. saprophyticus protein, or by modulating the transcription or translation of a nucleic acid molecule of the invention. In such methods, a microorganism expressing one or more proteins of the invention is contacted with one or more test compounds, and the effect of each test compound on the activity or level of expression of the protein is assessed.

VI. Screening Assays

(A) Primary Methods for Screening

The invention provides a method (also referred to herein as a “screening assay”) for identifying modulators, i.e., candidate or test compounds or agents (e.g., peptides, peptidomimetics, small molecules or other drugs) which bind to S. saprophyticus polypeptides, have a modulatory, e.g., inhibitory effect on, for example, S. saprophyticus polynucleotide expression or S. saprophyticus polypeptide activity, or have a modulatory, e.g., inhibitory effect on, for example, the expression or activity of S. saprophyticus polypeptide substrate, e.g., an anti-bacterial agent. In one embodiment, the agent eliminates infection, e.g., a S. saprophyticus infection, e.g., in a host. In another embodiment, the agent inhibits S. saprophyticus growth.

In one embodiment, the invention provides assays for screening candidate or test compounds which are substrates of an S. Saprophyticus polynucleotide or polypeptide or biologically active portion thereof. In another embodiment, the invention provides assays for screening candidate or test compounds which bind to or modulate the expression or activity of an S. Saprophyticus polynucleotide or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412421), or on beads (Lam (1991) Nature 354:82-84), chips (Fodor (1993) Nature 364:555-556), bacteria (Ladner U.S. Pat. No. 5,223,409), spores (Ladner USP'409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In one embodiment, an assay is a cell-based assay in which a cell which expresses an S. Saprophyticus polypeptide or biologically active portion thereof is contacted with a test compound and the ability of the test compound to modulate S. saprophyticus polypeptide activity is determined.

The ability of the test compound to modulate S. saprophyticus polypeptide binding to a substrate or to bind to an S. saprophyticus polypeptide can also be determined. Determining the ability of the test compound to modulate S. saprophyticus polypeptide binding to a substrate can be accomplished, for example, by coupling the S. saprophyticus polypeptide substrate with a radioisotope or enzymatic label such that binding of the S. saprophyticus polypeptide to S. saprophyticus polypeptide can be determined by detecting the labeled S. saprophyticus polypeptide substrate in a complex. Alternatively, an S. saprophyticus polypeptide could be coupled with a radioisotope or enzymatic label to monitor the ability of a test compound to modulate S. saprophyticus polypeptide binding to an S. saprophyticus polypeptide substrate in a complex. Determining the ability of the test compound to bind to an S. saprophyticus polypeptide can be accomplished, for example, by coupling the compound with a radioisotope or enzymatic label such that binding of the compound to an S. saprophyticus polypeptide can be determined by detecting the labeled S. saprophyticus compound in a complex. For example, compounds (e.g., S. saprophyticus polypeptide substrate or small molecule modulators, e.g., inhibitors) can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g., an S. saprophyticus polypeptide substrate) to interact with S. saprophyticus polypeptide without the labeling of any of the interactants. For example, a microphysiometer can be used to detect the interaction of a compound with S. saprophyticus polypeptide without the labeling of either the compound or the S. saprophyticus polypeptide. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound, e.g., a small molecule, and an S. saprophyticus polypeptide.

In another embodiment, an assay is a cell-based assay comprising contacting a cell expressing an S. saprophyticus polypeptide target molecule (e.g., an S. saprophyticus polypeptide substrate) with a test compound and determining the ability of the test compound to modulate (e.g., stimulate or inhibit) the activity of the S. saprophyticus polypeptide target molecule. Determining the ability of the test compound to modulate the activity of an S. saprophyticus polypeptide target molecule can be accomplished, for example, by determining the ability of the S. saprophyticus polypeptide to bind to or interact with the S. saprophyticus polypeptide target molecule.

Determining the ability of the S. saprophyticus polypeptide, or a biologically active fragment thereof, to bind to or interact with an S. saprophyticus polypeptide target molecule can be accomplished by one of the methods described above for determining direct binding. In a preferred embodiment, determining the ability of the S. saprophyticus polypeptide to bind to or interact with an S. saprophyticus polypeptide target molecule can be accomplished by determining the activity of the target molecule. For example, the activity of the target molecule can be determined by detecting induction of a cellular second messenger of the target (i.e., intra-cellular Ca²⁺, diacylglycerol, IP₃, and the like), detecting catalytic/enzymatic activity of the target using an appropriate substrate, detecting the induction of a reporter gene (comprising a target-responsive regulatory element operatively linked to a nucleic acid encoding a detectable marker, e.g., luciferase), or detecting a target-regulated cellular response.

In yet another embodiment, an assay of the present invention is a cell-free assay in which an S. saprophyticus polypeptide or biologically active portion thereof is contacted with a test compound, e.g., small molecule, and the ability of the test compound to bind to the S. saprophyticus polypeptide or biologically active portion thereof is determined. Preferred biologically active portions of the S. saprophyticus polypeptides to be used in assays of the present invention include fragments which have a role in the initiation or progression of infection. Binding of the test compound to the S. saprophyticus polypeptide can be determined either directly or indirectly as described above. In a preferred embodiment, the assay includes contacting the S. saprophyticus polypeptide or biologically active portion thereof with a known compound which binds S. saprophyticus polypeptide to form an assay mixture, contacting the assay mixture with a test compound, , e.g., a small molecule, and determining the ability of the test compound to interact with an S. saprophyticus polypeptide, wherein determining the ability of the test compound to interact with an S. saprophyticus polypeptide comprises determining the ability of the test compound to preferentially bind to S. saprophyticus polypeptide or biologically active portion thereof as compared to the known compound.

In another embodiment, the assay is a cell-free assay in which an S. saprophyticus polypeptide or biologically active portion thereof is contacted with a test compound and the ability of the test compound to modulate (e.g., stimulate or inhibit) the activity of the S. saprophyticus polypeptide or biologically active portion thereof is determined. Determining the ability of the test compound to modulate the activity of an S. saprophyticus polypeptide can be accomplished, for example, by determining the ability of the S. saprophyticus polypeptide to bind to an S. saprophyticus polypeptide target molecule by one of the methods described above for determining direct binding. Determining the ability of the S. saprophyticus polypeptide to bind to an S. saprophyticus polypeptide target molecule can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In an alternative embodiment, determining the ability of the test compound to modulate the activity of an S. saprophyticus polypeptide can be accomplished by determining the ability of the S. saprophyticus polypeptide to further modulate the activity of a downstream effector of an S. saprophyticus polypeptide target molecule. For example, the activity of the effector molecule on an appropriate S. saprophyticus target can be determined or the binding of the effector to an appropriate S. saprophyticus target can be determined as previously described.

In yet another embodiment, the cell-free assay involves contacting an S. saprophyticus polypeptide or biologically active portion thereof with a known compound which binds the S. saprophyticus polypeptide to form an assay mixture, contacting the assay mixture with a test compound, e.g., a small molecule, and determining the ability of the test compound to interact with the S. saprophyticus polypeptide, wherein determining the ability of the test compound to interact with the S. saprophyticus polypeptide comprises determining the ability of the S. saprophyticus polypeptide to preferentially bind to or modulate the activity of an S. saprophyticus polypeptide target molecule. In more than one embodiment of the above assay methods of the present invention, it may be desirable to immobilize either an S. saprophyticus polypeptide or its target molecule to facilitate separation of complexed from uncomplexed forms of one or both of the proteins, as well as to accommodate automation of the assay. Binding of a test compound to an S. saprophyticus polypeptide, or interaction of an S. saprophyticus polypeptide with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/S. saprophyticus fusion proteins or glutathione-S-transferase/target fusion proteins can be adsorbed onto glutathione sepharose beads (Sigma Chemical, St. Louis, Mo.) or glutathione derivatized microtiter plates, which are then combined with the test compound or the test compound and either the non-adsorbed target protein or S. saprophyticus polypeptide, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of an S. saprophyticus polypeptide binding or activity determined using standard techniques.

Other techniques for immobilizing proteins on matrices can also be used in the screening assays of the invention. For example, either an S. saprophyticus polypeptide or an S. saprophyticus polypeptide target molecule can be immobilized utilizing conjugation of biotin and streptavidin. Biotinylated S. saprophyticus polypeptide or target molecules can be prepared from biotin-NHS(N-hydroxy-succinimide)using techniques known in the art (e.g., biotinylation kit, Pierce Chemicals, Rockford, Ill.), and immobilized in the wells of streptavidin-coated 96 well plates (Pierce Chemical). Alternatively, antibodies reactive with S. saprophyticus polypeptide or target molecules but which do not interfere with binding of the S. saprophyticus polypeptide to its target molecule can be derivatized to the wells of the plate, and unbound target or S. saprophyticus polypeptide trapped in the wells by antibody conjugation. Methods for detecting such complexes, in addition to those described above for the GST-immobilized complexes, include immunodetection of complexes using antibodies reactive with the S. saprophyticus polypeptide or target molecule, as well as enzyme-linked assays which rely on detecting an enzymatic activity associated with the S. saprophyticus polypeptide or target molecule.

In another embodiment, modulators of S. saprophyticus polypeptide expression are identified in a method wherein a cell is contacted with a candidate compound and the expression of S. saprophyticus mRNA or polypeptide in the cell is determined. The level of expression of S. saprophyticus mRNA or polypeptide in the presence of the candidate compound is compared to the level of expression of S. saprophyticus mRNA or polypeptide in the absence of the candidate compound. The candidate compound can then be identified as a modulator of S. saprophyticus polypeptides expression based on this comparison. For example, when expression of S. saprophyticus mRNA or polypeptide is greater (statistically significantly greater) in the presence of the candidate compound than in its absence, the candidate compound is identified as a stimulator of S. saprophyticus mRNA or polypeptide expression. Alternatively, when expression of S. saprophyticus mRNA or polypeptide is less (statistically significantly less) in the presence of the candidate compound than in its absence, the candidate compound is identified as an inhibitor of S. saprophyticus mRNA or polypeptide expression. The level of S. saprophyticus mRNA or polypeptide expression in the cells can be determined by methods described herein for detecting S. saprophyticus mRNA or polypeptide.

In yet another aspect of the invention, the S. saprophyticus polypeptides can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with S. saprophyticus polypeptide (“S. saprophyticus polypeptide-binding proteins” or “S. saprophyticus polypeptide-bp”) and are involved in S. saprophyticus polypeptide activity. Such S. saprophyticus polypeptide-binding proteins are also likely to be involved in the propagation of signals by the S. saprophyticus polypeptides or S. saprophyticus polypeptide targets as, for example, downstream elements of an S. saprophyticus polypeptide-mediated signaling pathway. Alternatively, such S. saprophyticus polypeptide-binding proteins are likely to be S. saprophyticus polypeptide inhibitors.

The two-hybrid system is based on the modular nature of most transcription factors, which consist of separable DNA-binding and activation domains. Briefly, the assay utilizes two different DNA constructs. In one construct, the gene that codes for an S. saprophyticus polypeptide is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an S. saprophyticus polypeptide-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the S. saprophyticus polypeptide.

In another aspect, the invention pertains to a combination of two or more of the assays described herein. For example, a modulating agent can be identified using a cell-based or a cell free assay, and the ability of the agent to modulate the activity of an S. saprophyticus polypeptide can be confirmed in vivo, e.g., in an animal such as an animal model for a S. saprophyticus-associated disease or disorder, e.g., a urinary tract infection.

This invention further pertains to novel agents, e.g., small molecules, identified by the above-described screening assays. Accordingly, it is within the scope of this invention to further use an agent identified as described herein in an appropriate animal model. For example, an agent identified as described herein (e.g., an S. saprophyticus polypeptide modulating agent, an antisense S. saprophyticus nucleic acid molecule, an S. saprophyticus-specific antibody, or an S. saprophyticus polypeptide-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

Furthermore, the novel agents, e.g., small molecules, identified by the screening assays as described herein, but be further tested in a non-animal model assay, e.g., a DNA microarray, or a radioactive precursor incorporation assay.

(B) Secondary Screening of Polypeptides and Analogs

The high through-put assays described above can be followed by secondary screens in order to identify further biological activities which will, e.g., allow one skilled in the art to differentiate agonists from antagonists. The type of a secondary screen used will depend on the desired activity that needs to be tested. For example, an assay can be developed in which the ability to inhibit an interaction between a protein of interest and its respective ligand can be used to identify antagonists from a group of peptide fragments isolated though one of the primary screens described above.

Therefore, methods for generating fragments and analogs and testing them for activity are known in the art. Once the core sequence of interest is identified, it is routine for one skilled in the art to obtain analogs and fragments.

(C) Peptide Mimetics of S. saprophyticus Polypeptides

The invention also provides for reduction of the protein binding domains of the subject S. saprophyticus polypeptides to generate mimetics, e.g. peptide or non-peptide agents. The peptide mimetics are able to disrupt binding of a polypeptide to its counter ligand, e.g., in the case of an S. saprophyticus polypeptide binding to a naturally occurring ligand. The critical residues of a subject S. saprophyticus polypeptide which are involved in molecular recognition of a polypeptide can be determined and used to generate S. saprophyticus-derived peptidomimetics which competitively or noncompetitively inhibit binding of the S. saprophyticus polypeptide with an interacting polypeptide (see, for example, European patent applications EP-412,762A and EP-B31,080A).

For example, scanning mutagenesis can be used to map the amino acid residues of a particular S. saprophyticus polypeptide involved in binding an interacting polypeptide, peptidomimetic compounds (e.g. diazepine or isoquinoline derivatives) can be generated which mimic those residues in binding to an interacting polypeptide, and which therefore can inhibit binding of an S. saprophyticus polypeptide to an interacting polypeptide and thereby interfere with the function of S. saprophyticus polypeptide. For instance, non-hydrolyzable peptide analogs of such residues can be generated using benzodiazepine (e.g., see Freidinger et al. in Peptides: Chemistry and Biology, G. R. Marshall ed., ESCOM Publisher: Leiden, Netherlands, 1988), azepine (e.g., see Huffinan et al. in Peptides: Chemistry and Biology, G. R. Marshall ed., ESCOM Publisher: Leiden, Netherlands, 1988), substituted gama lactam rings (Garvey et al. in Peptides: Chemistry and Biology, G. R. Marshall ed., ESCOM Publisher: Leiden, Netherlands, 1988), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), β-turn dipeptide cores (Nagai et al. (1985) Tetrahedron Lett 26:647; and Sato et al. (1986) J Chem Soc Perkin Trans 1:1231), and β-aminoalcohols (Gordon et al. (1985) Biochem Biophys Res Commun 126:419; and Dann et al. (1986) Biochem Biophys Res Commun 134:71).

VII. Identification of Nucleic Acids Encoding Vaccine Components and Targets for Agents Effective Azainst S. saprophyticus

The disclosed S. saprophyticus genome sequence includes segments that direct the synthesis of ribonucleic acids and polypeptides, as well as origins of replication, promoters, other types of regulatory sequences, and intergenic nucleic acids. The invention encompasses nucleic acids encoding immunogenic components of vaccines and targets for agents effective against S. saprophyticus. Identification of said immunogenic components involved in the determination of the function of the disclosed sequences can be achieved using a variety of approaches. Non-limiting examples of these approaches are described briefly below.

(A) Homology to known sequences: Computer-assisted comparison of the disclosed S. saprophyticus sequences with previously reported sequences present in publicly available databases is useful for identifying functional S. saprophyticus nucleic acid and polypeptide sequences. It will be understood that protein-coding sequences, for example, may be compared as a whole, and that a high degree of sequence homology between two proteins (such as, for example, >80-90%) at the amino acid level indicates that the two proteins also possess some degree of functional homology, such as, for example, among enzymes involved in metabolism, DNA synthesis, or cell wall synthesis, and proteins involved in transport, cell division, etc. In addition, many structural features of particular protein classes have been identified and correlate with specific consensus sequences, such as, for example, binding domains for nucleotides, DNA, metal ions, and other small molecules; sites for covalent modifications such as phosphorylation, acylation, and the like; sites of protein:protein interactions, etc. These consensus sequences may be quite short and thus may represent only a fraction of the entire protein-coding sequence. Identification of such a feature in an S. saprophyticus sequence is therefore useful in determining the function of the encoded protein and identifying useful targets of antibacterial drugs. S. saprophyticus proteins identified as containing putative signal sequences and/or transmembrane domains are useful as immunogenic components of vaccines.

(B) Identification of essential genes: Nucleic acids that encode proteins essential for growth or viability of S. saprophyticus are preferred drug targets. S. saprophyticus genes can be tested for their biological relevance to the organism by examining the effect of deleting and/or disrupting the genes, i.e., by so-called gene “knockout”, using techniques known to those skilled in the relevant art. In this manner, essential genes may be identified.

(C) Strain-specific sequences: Because of the evolutionary relationship between different S. saprophyticus strains, it is believed that the presently disclosed S. saprophyticus sequences are useful for identifying, and/or discriminating between, previously known and new S. saprophyticus strains. It is believed that other S. saprophyticus strains will exhibit at least 70%, 80%, 90% or more sequence homology with the presently disclosed sequence. Systematic and routine analyses of DNA sequences derived from samples containing S. saprophyticus strains, and comparison with the present sequence allows for the identification of sequences that can be used to discriminate between strains, as well as those that are common to all S. saprophyticus strains. In one embodiment, the invention provides nucleic acids, including probes, and peptide and polypeptide sequences that discriminate between different strains of S. saprophyticus. Strain-specific components can also be identified functionally by their ability to elicit or react with antibodies that selectively recognize one or more S. saprophyticus strains.

In another embodiment, the invention provides nucleic acids, including probes, and peptide and polypeptide sequences that are common to all S. saprophyticus strains but are not found in other bacterial species.

In another embodiment, the nucleic acids, including probes, and peptide and polypeptide sequences of the invention provide epidemiological utility to further understand the spread of disease.

(D) Specific Example: Determination Of Candidate Protein Antigens For Antibody And Vaccine Development

The selection of candidate protein antigens for vaccine development can be derived from the nucleic acids encoding S. saprophyticus polypeptides. First, the ORF's can be analyzed for homology to other known exported or membrane proteins and analyzed using the discriminant analysis described by Klein, et al. (Klein, P., Kanehsia, M., and DeLisi, C. (1985) Biochimica et Biophysica Acta 815, 468-476) for predicting exported and membrane proteins.

Homology searches can be performed using the BLAST algorithm contained in the Wisconsin Sequence Analysis Package (Genetics Computer Group, University Research Park, 575 Science Drive, Madison, Wis. 53711) to compare each predicted ORF amino acid sequence with all sequences found in the current GenBank, SWISS-PROT and PIR databases. BLAST searches for local alignments between the ORF and the databank sequences and reports a probability score which indicates the probability of finding this sequence by chance in the database. ORF's with significant homology (e.g. probabilities lower than 1×10⁻⁶ indicate that the homology is only due to random chance) to membrane or exported proteins represent protein antigens for vaccine development. Possible functions can be provided to S. saprophyticus genes based on sequence homology to genes cloned in other organisms, e.g., S. epidermitis (as set forth in FIG. 1).

Discriminant analysis (Klein, et al. supra) can be used to examine the ORF amino acid sequences. This algorithm uses the intrinsic information contained in the ORF amino acid sequence and compares it to information derived from the properties of known membrane and exported proteins. This comparison predicts which proteins will be exported, membrane associated or cytoplasmic. ORF amino acid sequences identified as exported or membrane associated by this algorithm are likely protein antigens for vaccine development.

Infrequently it is not possible to distinguish between multiple possible nucleotides at a given position in the nucleic acid sequence. In those cases the ambiguities are denoted by an extended alphabet as follows:

These are the official IUPAC-IUB single-letter base codes

| Code | Base Description | |
|---|---|---|
| G | Guanine | |
| A | Adenine | |
| T | Thymine | |
| C | Cytosine | |
| R | Purine | (A or G) |
| Y | Pyrimidine | (C or T or U) |
| M | Amino | (A or C) |
| K | Ketone | (G or T) |
| S | Strong interaction | (C or G) |
| W | Weak interaction | (A or T) |
| H | Not-G | (A or C or T) |
| B | Not-A | (C or G or T) |
| V | Not-T (not-U) | (A or C or G) |
| D | Not-C | (A or G or T) |
| N | Any | (A or C or G or T) |

The amino acid translations of this invention account for the ambiguity in the nucleic acid sequence by translating the ambiguous codon as the letter “X”. In all cases, the permissible amino acid residues at a position are clear from an examination of the nucleic acid sequence based on the standard genetic code.

VIII. Production of Fragments and Analogs of S. saprophyticus Nucleic Acids and Polypeptides

Based on the discovery of the S. saprophyticus gene products of the invention provided in FIG. 1, one skilled in the art can alter the disclosed structure (of S. saprophyticus genes), e.g., by producing fragments or analogs, and test the newly produced structures for activity. Examples of techniques known to those skilled in the relevant art which allow the production and testing of fragments and analogs are discussed below. These, or analogous methods can be used to make and screen libraries of polypeptides, e.g., libraries of random peptides or libraries of fragments or analogs of cellular proteins for the ability to bind S. saprophyticus polypeptides. Such screens are useful for the identification of inhibitors of S. saprophyticus, as described herein.

(A) Generation of Fragments

Fragments of a protein of the invention can be produced in several ways, e.g., recombinantly, by proteolytic digestion, or by chemical synthesis. Internal or terminal fragments of a polypeptide can be generated by removing one or more nucleotides from one end (for a terminal fragment) or both ends (for an internal fragment) of a nucleic acid which encodes the polypeptide. Expression of the mutagenized DNA produces polypeptide fragments. Digestion with “end-nibbling” endonucleases can thus generate DNA's which encode an array of fragments. DNA's which encode fragments of a protein can also be generated by random shearing, restriction digestion or a combination of the above-discussed methods.

Fragments can also be chemically synthesized using techniques known in the art such as conventional Merrifield solid phase f-Moc or t-Boc chemistry. For example, peptides of the present invention may be arbitrarily divided into fragments of desired length with no overlap of the fragments, or divided into overlapping fragments of a desired length.

(B) Alteration of Nucleic Acids and Polypeptides: Random Methods

Amino acid sequence variants of a protein of the invention can be prepared by random mutagenesis of DNA which encodes a protein or a particular domain or region of a protein. Useful methods include PCR mutagenesis and saturation mutagenesis. A library of random amino acid sequence variants can also be generated by the synthesis of a set of degenerate oligonucleotide sequences. (Methods for screening proteins in a library of variants are elsewhere herein).

(i) PCR Mutagenesis

In PCR mutagenesis, reduced Taq polymerase fidelity is used to introduce random mutations into a cloned fragment of DNA (Leung et al., 1989, Technique 1:11-15). The DNA region to be mutagenized is amplified using the polymerase chain reaction (PCR) under conditions that reduce the fidelity of DNA synthesis by Taq DNA polymerase, e.g., by using a dGTP/dATP ratio of five and adding Mn²⁺ to the PCR reaction. The pool of amplified DNA fragments are inserted into appropriate cloning vectors to provide random mutant libraries.

(ii) Saturation Mutagenesis

Saturation mutagenesis allows for the rapid introduction of a large number of single base substitutions into cloned DNA fragments (Mayers et al., 1985, Science 229:242). This technique includes generation of mutations, e.g., by chemical treatment or irradiation of single-stranded DNA in vitro, and synthesis of a complimentary DNA strand. The mutation frequency can be modulated by modulating the severity of the treatment, and essentially all possible base substitutions can be obtained. Because this procedure does not involve a genetic selection for mutant fragments both neutral substitutions, as well as those that alter function, are obtained. The distribution of point mutations is not biased toward conserved sequence elements.

(iii) Degenerate Oligonucleotides

A library of homologs can also be generated from a set of degenerate oligonucleotide sequences. Chemical synthesis of a degenerate sequences can be carried out in an automatic DNA synthesizer, and the synthetic genes then ligated into an appropriate expression vector. The synthesis of degenerate oligonucleotides is known in the art (see for example, Narang, SA (1983) Tetrahedron 39:3; Itakura et al. (1981) Recombinant DNA, Proc 3rd Cleveland Sympos. Macromolecules, ed. A G Walton, Amsterdam: Elsevier pp 273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

(C) Alteration of Nucleic Acids and Polypeptides: Methods for Directed Mutagenesis

Non-random or directed, mutagenesis techniques can be used to provide specific sequences or mutations in specific regions. These techniques can be used to create variants which include, e.g., deletions, insertions, or substitutions, of residues of the known amino acid sequence of a protein. The sites for mutation can be modified individually or in series, e.g., by (1) substituting first with conserved amino acids and then with more radical choices depending upon results achieved, (2) deleting the target residue, or (3) inserting residues of the same or a different class adjacent to the located site, or combinations of options 1-3.

(i) Alanine Scanning Mutagenesis

Alanine scanning mutagenesis is a useful method for identification of certain residues or regions of the desired protein that are preferred locations or domains for mutagenesis, Cunningham and Wells (Science 244:1081-1085, 1989). In alanine scanning, a residue or group of target residues are identified (e.g., charged residues such as Arg, Asp, His, Lys, and Glu) and replaced by a neutral or negatively charged amino acid (most preferably alanine or polyalanine). Replacement of an amino acid can affect the interaction of the amino acids with the surrounding aqueous environment in or outside the cell. Those domains demonstrating functional sensitivity to the substitutions are then refined by introducing further or other variants at or for the sites of substitution. Thus, while the site for introducing an amino acid sequence variation is predetermined, the nature of the mutation per se need not be predetermined. For example, to optimize the performance of a mutation at a given site, alanine scanning or random mutagenesis may be conducted at the target codon or region and the expressed desired protein subunit variants are screened for the optimal combination of desired activity.

(ii) Oligonucleotide-Mediated Mutagenesis

Oligonucleotide-mediated mutagenesis is a useful method for preparing substitution, deletion, and insertion variants of DNA, see, e.g., Adelman et al., (DNA 2:183, 1983). Briefly, the desired DNA is altered by hybridizing an oligonucleotide encoding a mutation to a DNA template, where the template is the single-stranded form of a plasmid or bacteriophage containing the unaltered or native DNA sequence of the desired protein. After hybridization, a DNA polymerase is used to synthesize an entire second complementary strand of the template that will thus incorporate the oligonucleotide primer, and will code for the selected alteration in the desired protein DNA. Generally, oligonucleotides of at least 25 nucleotides in length are used. An optimal oligonucleotide will have 12 to 15 nucleotides that are completely complementary to the template on either side of the nucleotide(s) coding for the mutation. This ensures that the oligonucleotide will hybridize properly to the single-stranded DNA template molecule. The oligonucleotides are readily synthesized using techniques known in the art such as that described by Crea et al. (Proc. Natl. Acad. Sci. USA, 75: 5765[1978]).

(iii) Cassette Mutagenesis

Another method for preparing variants, cassette mutagenesis, is based on the technique described by Wells et al. (Gene, 34:315[1985]). The starting material is a plasmid (or other vector) which includes the protein subunit DNA to be mutated. The codon(s) in the protein subunit DNA to be mutated are identified. There must be a unique restriction endonuclease site on each side of the identified mutation site(s). If no such restriction sites exist, they may be generated using the above-described oligonucleotide-mediated mutagenesis method to introduce them at appropriate locations in the desired protein subunit DNA. After the restriction sites have been introduced into the plasmid, the plasmid is cut at these sites to linearize it. A double-stranded oligonucleotide encoding the sequence of the DNA between the restriction sites but containing the desired mutation(s) is synthesized using standard procedures. The two strands are synthesized separately and then hybridized together using standard techniques. This double-stranded oligonucleotide is referred to as the cassette. This cassette is designed to have 3′ and 5′ ends that are comparable with the ends of the linearized plasmid, such that it can be directly ligated to the plasmid. This plasmid now contains the mutated desired protein subunit DNA sequence.

(iv) Combinatorial Mutagenesis

Combinatorial mutagenesis can also be used to generate mutants (Ladner et al., WO 88/06630). In this method, the amino acid sequences for a group of homologs or other related proteins are aligned, preferably to promote the highest homology possible. All of the amino acids which appear at a given position of the aligned sequences can be selected to create a degenerate set of combinatorial sequences. The variegated library of variants is generated by combinatorial mutagenesis at the nucleic acid level, and is encoded by a variegated gene library. For example, a mixture of synthetic oligonucleotides can be enzymatically ligated into gene sequences such that the degenerate set of potential sequences are expressible as individual peptides, or alternatively, as a set of larger fusion proteins containing the set of degenerate sequences.

(D) Other Modifications of S. saprophyticus Nucleic Acids and Polypeptides

It is possible to modify the structure of an S. saprophyticus polypeptide for such purposes as increasing solubility, enhancing stability (e.g., shelf life ex vivo and resistance to proteolytic degradation in vivo). A modified S. saprophyticus protein or peptide can be produced in which the amino acid sequence has been altered, such as by amino acid substitution, deletion, or addition as described herein.

An S. saprophyticus peptide can also be modified by substitution of cysteine residues preferably with alanine, serine, threonine, leucine or glutamic acid residues to minimize dimerization via disulfide linkages. In addition, amino acid side chains of fragments of the protein of the invention can be chemically modified. Another modification is cyclization of the peptide.

In order to enhance stability and/or reactivity, an S. saprophyticus polypeptide can be modified to incorporate one or more polymorphisms in the amino acid sequence of the protein resulting from any natural allelic variation. Additionally, D-amino acids, non-natural amino acids, or non-amino acid analogs can be substituted or added to produce a modified protein within the scope of this invention. Furthermore, an S. saprophyticus polypeptide can be modified using polyethylene glycol (PEG) according to the method of A. Sehon and co-workers (Wie et al., supra) to produce a protein conjugated with PEG. In addition, PEG can be added during chemical synthesis of the protein. Other modifications of S. saprophyticus proteins include reduction/alkylation (Tarr, Methods of protein Microcharacterization, J. E. Silver ed., Humana Press, Clifton N.J. 155-194 (1986)); acylation (Tarr, supra); chemical coupling to an appropriate carrier (Mishell and Shiigi, eds, Selected Methods in Cellular Immunology, W H Freeman, San Francisco, Calif. (1980), U.S. Pat. No. 4,939,239; or mild formalin treatment (Marsh, (1971) Int. Arch. of Allergy and Appl. Immunol., 41: 199-215).

To facilitate purification and potentially increase solubility of an S. saprophyticus protein or peptide, it is possible to add an amino acid fusion moiety to the peptide backbone. For example, hexa-histidine can be added to the protein for purification by immobilized metal ion affinity chromatography (Hochuli, E. et al., (1988) Bio/Technology, 6: 1321-1325). In addition, to facilitate isolation of peptides free of irrelevant sequences, specific endoprotease cleavage sites can be introduced between the sequences of the fusion moiety and the peptide.

To potentially aid proper antigen processing of epitopes within an S. saprophyticus polypeptide, canonical protease sensitive sites can be engineered between regions, each comprising at least one epitope via recombinant or synthetic methods. For example, charged amino acid pairs, such as KK or RR, can be introduced between regions within a protein or fragment during recombinant construction thereof. The resulting peptide can be rendered sensitive to cleavage by cathepsin and/or other trypsin-like enzymes which would generate portions of the protein containing one or more epitopes. In addition, such charged amino acid residues can result in an increase in the solubility of the peptide.

IX. Vaccine Formulations for S. saprophyticus Nucleic Acids and Polypeptides

This invention also features vaccine compositions or formulations (used interchangeably herein) for protection against infection by S. saprophyticus or for treatment of S. saprophyticus infection. As used herein, the term “treatment of S. saprophyticus-associated disease or disorder” refers to therapeutic treatment of an existing or established S. saprophyticus-associated disease or disorder. The terms “protection against S. saprophyticus-associated disease or disorder”or “prophylactic treatment” refer to the use of S. saprophyticus vaccine formulation for reducing the risk of or preventing an infection in a subject at risk for S. saprophyticus -associated disease or disorder. In one embodiment, the vaccine compositions contain one or more immunogenic components, such as a surface protein, from S. saprophyticus, or portion thereof, and a pharmaceutically acceptable carrier. For example, in one embodiment, the vaccine formulations of the invention contain at least one or combination of S. saprophyticus polypeptides or fragments thereof, from same or different S. saprophyticus antigens. Nucleic acids and S. saprophyticus polypeptides for use in the vaccine formulations of the invention include the nucleic acids and polypeptides set forth in FIG. 1 and B, preferably those S. saprophyticus nucleic acids that encode surface proteins and surface proteins or fragments thereof. However, any nucleic acid encoding an immunogenic S. saprophyticus protein and S. saprophyticus polypetide, or portion thereof, can be used in the present invention. These vaccines have therapeutic and/or prophylactic utilities.

One aspect of the invention provides a vaccine composition for protection against infection by S. saprophyticus which contains at least one immunogenic fragment of an S. saprophyticus protein and a pharmaceutically acceptable carrier. Preferred fragments include peptides of at least about 10 amino acid residues in length, preferably about 10, 11, 12, 13, 14, 15, 16, 17, 18, 19, 20 amino acid residues in length, and more preferably about 12-16 amino acid residues in length.

Immunogenic components of the invention can be obtained, for example, by screening polypeptides recombinantly produced from the corresponding fragment of the nucleic acid encoding the full-length S. saprophyticus protein. In addition, fragments can be chemically synthesized using techniques known in the art such as conventional Merrifield solid phase f-Moc or t-Boc chemistry.

In one embodiment, immunogenic components are identified by the ability of the peptide to stimulate T cells. Peptides which stimulate T cells, as determined by, for example, T cell proliferation or cytokine secretion are defined herein as comprising at least one T cell epitope. T cell epitopes are believed to be involved in initiation and perpetuation of the immune response to the protein immunogen. These T cell epitopes are thought to trigger early events at the level of the T helper cell by binding to an appropriate HLA molecule on the surface of an antigen presenting cell, thereby stimulating the T cell subpopulation with the relevant T cell receptor for the epitope. These events lead to T cell proliferation, lymphokine secretion, local inflammatory reactions, recruitment of additional immune cells to the site of antigen/T cell interaction, and activation of the B cell cascade, leading to the production of antibodies. A T cell epitope is the basic element, or smallest unit of recognition by a T cell receptor, where the epitope comprises amino acids essential to receptor recognition (e.g., approximately 6 or 7 amino acid residues). Amino acid sequences which mimic those of the T cell epitopes are within the scope of this invention.

In another embodiment, immunogenic components of the invention are identified through genomic vaccination. The basic protocol is based on the idea that expression libraries consisting of all or parts of a pathogen genome, e.g., an S. saprophyticus genome, can confer protection when used to genetically immunize a host. This expression library immunization (ELI) is analogous to expression cloning and involves reducing a genomic expression library of a pathogen, e.g., S. saprophyticus, into plasmids that can act as genetic vaccines. The plasmids can also be designed to encode genetic adjuvants which can dramatically stimulate the humoral response. These genetic adjuvants can be introduced at remote sites and act as well extracelluraly as intracellularly.

This is a new approach to vaccine production that has many of the advantages of live/attenuated pathogens but no risk of infection. An expression library of pathogen DNA is used to immunize a host thereby producing the effects of antigen presentation of a live vaccine without the risk. For example, in the present invention, random fragments from the S. saprophyticus genome or from cosmid or plasmid clones, as well as PCR products from genes identified by genomic sequencing, can be used to immunize a host. The feasibility of this approach has been demonstrated with Mycoplasma pulmonis (Barry et al., Nature 377:632-635, 1995), where even partial expression libraries of Mycoplasma pulmonis, a natural pathogen in rodents, provided protection against challenge from the pathogen.

ELI is a technique that allows for production of a non-infectious multipartite vaccine, even when little is known about pathogen's biology, because ELI uses the immune system to screen candidate genes. Once isolated, these genes can be used as genetic vaccines or for development of recombinant protein vaccines. Thus, ELI allows for production of vaccines in a systematic, largely mechanized fashion.

Screening immunogenic components can be accomplished using one or more of several different assays. For example, in vitro, peptide T cell stimulatory activity is assayed by contacting a peptide known or suspected of being immunogenic with an antigen presenting cell which presents appropriate MHC molecules in a T cell culture. Presentation of an immunogenic S. saprophyticus peptide in association with appropriate MHC molecules to T cells in conjunction with the necessary costimulation has the effect of transmitting a signal to the T cell that induces the production of increased levels of cytokines, particularly of interleukin-2 and interleukin-4. The culture supernatant can be obtained and assayed for interleukin-2 or other known cytokines. For example, any one of several conventional assays for interleukin-2 can be employed, such as the assay described in Proc. Natl. Acad. Sci USA, 86: 1333 (1989) the pertinent portions of which are incorporated herein by reference. A kit for an assay for the production of interferon is also available from Genzyme Corporation (Cambridge, Mass.).

Alternatively, a common assay for T cell proliferation entails measuring tritiated thymidine incorporation. The proliferation of T cells can be measured in vitro by determining the amount of ³H-labeled thymidine incorporated into the replicating DNA of cultured cells. Therefore, the rate of DNA synthesis and, in turn, the rate of cell division can be quantified.

Vaccine compositions or formulations of the invention containing one or more immunogenic components (e.g., S. saprophyticus polypeptide or fragment thereof or nucleic acid encoding an S. saprophyticus polypeptide or fragment thereof) preferably include a pharmaceutically acceptable carrier. The term “pharmaceutically acceptable carrier” is intended to include any and all solvents, dispersion media, coatings, antibacterial and antifungal agents, isotonic and absorption delaying agents, and the like, compatible with pharmaceutical administration. Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the S. saprophyticus nucleic acid or polypeptide. For vaccine formulations of the invention containing S. saprophyticus polypeptides, the polypeptide is preferably coadministered with a suitable adjuvant and/or a delivery system described herein.

It will be apparent to those of skill in the art that the therapeutically effective amount of DNA or protein of this invention will depend, inter alia, upon the administration schedule, the unit dose of an S. saprophyticus nucleic acid or polypeptide administered, whether the protein or nucleic acid is administered in combination with other therapeutic agents, the immune status and health of the patient, and the therapeutic activity of the particular protein or nucleic acid.

Vaccine formulations are conventionally administered parenterally, e.g., by injection, either subcutaneously or intramuscularly. Methods for intramuscular immunization are described by Wolff et al. (1990) Science 247: 1465-1468 and by Sedegah et al. (1994) Immunology 91: 9866-9870. Other modes of administration include oral and pulmonary formulations, suppositories, and transdermal applications. Oral immunization is preferred over parenteral methods for inducing protection against infection by S. saprophyticus. Czinn et. al. (1993) Vaccine 11: 637-642. Oral formulations include such normally employed excipients as, for example, pharmaceutical grades of mannitol, lactose, starch, magnesium stearate, sodium saccharine, cellulose, magnesium carbonate, and the like.

In one embodiment, the vaccine formulation includes, as a pharmaceutically acceptable carrier, an adjuvant. Examples of the suitable adjuvants for use in the vaccine formulations of the invention include, but are not limited, to aluminum hydroxide; N-acetyl-muramyl-L-threonyl-D-isoglutamine (thr-MDP); N-acetyl-nor-muramyl-L-alanyl-D-isoglutamine (CGP 11637, referred to as nor-MDP); N-acetylmuramyl-L-alanyl-D-isoglutaminyl-L-alanine-2-(1′-2′-dipalmitoyl-sn-glycero-3-hydroxyphos-phoryloxy)-ethylamine (CGP 19835A, referred to a MTP-PE); RIBI, which contains three components from bacteria; monophosphoryl lipid A; trehalose dimycoloate; cell wall skeleton (MPL+TDM +CWS) in a 2% squalene/Tween 80 emulsion; and cholera toxin. Others which may be used are non-toxic derivatives of cholera toxin, including its B subunit, and/or conjugates or genetically engineered fusions of the S. saprophyticus polypeptide with cholera toxin or its B subunit, procholeragenoid, fungal polysaccharides, including schizophyllan, muramyl dipeptide, muramyl dipeptide derivatives, phorbol esters, labile toxin of E. coli, non-S. saprophyticus bacterial lysates, block polymers or saponins.

In another embodiment, the vaccine formulation includes, as a pharmaceutically acceptable carrier, a delivery system. Suitable delivery systems for use in the vaccine formulations of the invention include biodegradable microcapsules or immuno-stimulating complexes (ISCOMs), cochleates, or liposomes, genetically engineered attenuated live vectors such as viruses or bacteria, and recombinant (chimeric) virus-like particles, e.g., bluetongue. In another embodiment of the invention, the vaccine formulation includes both a delivery system and an adjuvant.

Delivery systems in humans may include enteric release capsules protecting the antigen from the acidic environment of the stomach, and including S. saprophyticus polypeptide in an insoluble form as fusion proteins. Suitable carriers for the vaccines of the invention are enteric coated capsules and polylactide-glycolide microspheres. Suitable diluents are 0.2 N NaHCO3 and/or saline.

Vaccines of the invention can be administered as a primary prophylactic agent in adults or in children, as a secondary prevention, after successful eradication of S. saprophyticus in an infected host, or as a therapeutic agent in the aim to induce an immune response in a susceptible host to prevent infection by S. saprophiticus. The vaccines of the invention are administered in amounts readily determined by persons of ordinary skill in the art. Thus, for adults a suitable dosage will be in the range of 10 μg to 10 g, preferably 10 μg to 100 mg, for example 50 μg to 50 mg. A suitable dosage for adults will also be in the range of 5 μg to 500 mg. Similar dosage ranges will be applicable for children.

The amount of adjuvant employed will depend on the type of adjuvant used. For example, when the mucosal adjuvant is cholera toxin, it is suitably used in an amount of 5 μg to 50 μg, for example 10 μg to 35 μg. When used in the form of microcapsules, the amount used will depend on the amount employed in the matrix of the microcapsule to achieve the desired dosage. The determination of this amount is within the skill of a person of ordinary skill in the art.

Those skilled in the art will recognize that the optimal dose may be more or less depending upon the patient's body weight, disease, the route of administration, and other factors. Those skilled in the art will also recognize that appropriate dosage levels can be obtained based on results with known oral vaccines such as, for example, a vaccine based on an E. coli lysate (6 mg dose daily up to total of 540 mg) and with an enterotoxigenic E. coli purified antigen (4 doses of 1 mg) (Schulman et al., J. Urol. 150:917-921 (1993)); Boedecker et al., American Gastroenterological Assoc. 999:A-222 (1993)). The number of doses will depend upon the disease, the formulation, and efficacy data from clinical trials. Without intending any limitation as to the course of treatment, the treatment can be administered over 3 to 8 doses for a primary immunization schedule over 1 month (Boedeker, American Gastroenterological Assoc. 888:A-222 (1993)).

In a preferred embodiment, a vaccine composition of the invention can be based on a killed whole E. coli preparation with an immunogenic fragment of an S. saprophyticus protein of the invention expressed on its surface or it can be based on an E. coli lysate, wherein the killed E. coli acts as a carrier or an adjuvant.

It will be apparent to those skilled in the art that some of the vaccine compositions of the invention are useful only for preventing S. saprophyticus infection, some are useful only for treating S. saprophyticus infection, and some are useful for both preventing and treating S. saprophyticus infection. In a preferred embodiment, the vaccine composition of the invention provides protection against S. saprophyticus infection by stimulating humoral and/or cell-mediated immunity against S. saprophyticus. It should be understood that amelioration of any of the symptoms of S. saprophyticus-associated disease or disorder is a desirable clinical goal, including a lessening of the dosage of medication used to treat an S. saprophyticus-associated disease or disorder, or an increase in the production of antibodies in the serum or mucosal surfaces, e.g., of the urogenital tract of a subject.

X. Antibodies Reactive With S. saprophyticus Polvpeptides

The invention also includes antibodies specifically reactive with the subject S. saprophyticus polypeptide. Anti-protein/anti-peptide antisera or monoclonal antibodies can be made by standard protocols (See, for example, Antibodies: A Laboratory Manual ed. by Harlow and Lane (Cold Spring Harbor Press: 1988)). A mammal such as a mouse, a hamster or rabbit can be immunized with an immunogenic form of the peptide. Techniques for conferring immunogenicity on a protein or peptide include conjugation to carriers or other techniques well known in the art. An immunogenic portion of the subject S. saprophyticus polypeptide can be administered in the presence of adjuvant. The progress of immunization can be monitored by detection of antibody titers in plasma or serum. Standard ELISA or other immunoassays can be used with the immunogen as antigen to assess the levels of antibodies.

In a preferred embodiment, the subject antibodies are immunospecific for antigenic determinants of the S. saprophyticus polypeptides of the invention, e.g. antigenic determinants of a polypeptide of the invention contained in FIG. 1, or a closely related human or non-human mammalian homolog (e.g., 70%, 75%, 80%, 85%, 90%, 91%, 92%, 93%, 94%, 95%, 96%, 97%, 98%, or 99% homologous). In yet a further preferred embodiment of the invention, the anti-S. saprophyticus antibodies do not substantially cross react (i.e., react specifically) with a protein which is for example, less than 70% percent homologous to a sequence of the invention contained in FIG. 1. By “not substantially cross react”, it is meant that the antibody has a binding affinity for a non-homologous protein which is less than 10 percent, more preferably less than 5 percent, and even more preferably less than 1 percent, of the binding affinity for a protein of the invention contained in FIG. 1. In a most preferred embodiment, there is no crossreactivity between bacterial and mammalian antigens.

In preferred embodiments, the antibodies specifically bind to the S. saprophyticus polypeptides of the invention or a fragment thereof. The terms “antibody” and “antibodies” as used interchangeably herein refer to immunoglobulin molecules as well as fragments and derivatives thereof that comprise an immunologically active portion of an immunoglobulin molecule, (i.e., such a portion contains an antigen binding site which specifically binds an antigen, such as an S. saprophyticus polypeptide). An antibody which specifically binds to a protein of the invention is an antibody which binds the protein, but does not substantially bind other molecules in a sample, e.g., a biological sample, which naturally contains the protein. Examples of an immunologically active portion of an immunoglobulin molecule include, but are not limited to, single-chain antibodies (scAb), F(ab) and F(ab′)2 fragments.

An isolated protein of the invention or a fragment thereof can be used as an immunogen to generate antibodies. The full-length protein can be used or, alternatively, the invention provides antigenic peptide fragments for use as immunogens. The antigenic peptide of a protein of the invention comprises at least 8 (preferably 10, 15, 20, or 30 or more) amino acid residues of the amino acid sequence of one of the proteins of the invention, and encompasses at least one epitope of the protein such that an antibody raised against the peptide forms a specific immune complex with the protein. Preferred epitopes encompassed by the antigenic peptide are regions that are located on the surface of the protein, e.g., hydrophilic regions. Hydrophobicity sequence analysis, hydrophilicity sequence analysis, or similar analyses can be used to identify hydrophilic regions. In preferred embodiments, an isolated marker protein or fragment thereof is used as an immunogen.

An immunogen typically is used to prepare antibodies by immunizing a suitable (i.e. immunocompetent) subject such as a rabbit, goat, mouse, or other mammal. An appropriate immunogenic preparation can contain, for example, recombinantly-expressed or chemically-synthesized protein or peptide. The preparation can further include an adjuvant, such as Freund's complete or incomplete adjuvant, or a similar immunostimulatory agent. Preferred immunogen compositions are those that contain no other human proteins such as, for example, immunogen compositions made using a non-human host cell for recombinant expression of a protein of the invention. In such a manner, the resulting antibody compositions have reduced or no binding of human proteins other than a protein of the invention.

The invention provides polyclonal and monoclonal antibodies. The term “monoclonal antibody” or “monoclonal antibody composition”, as used herein, refers to a population of antibody molecules that contain only one species of an antigen binding site capable of immunoreacting with a particular epitope. Preferred polyclonal and monoclonal antibody compositions are ones that have been selected for antibodies directed against a protein of the invention. Particularly preferred polyclonal and monoclonal antibody preparations are ones that contain only antibodies directed against a marker protein or fragment thereof.

Polyclonal antibodies can be prepared by immunizing a suitable subject with a protein of the invention as an immunogen The antibody titer in the immunized subject can be monitored over time by standard techniques, such as with an enzyme linked immunosorbent assay (ELISA) using immobilized polypeptide. At an appropriate time after immunization, e.g., when the specific antibody titers are highest, antibody-producing cells can be obtained from the subject and used to prepare monoclonal antibodies (mAb) by standard techniques, such as the hybridoma technique originally described by Kohler and Milstein (1975) Nature 256:495497, the human B cell hybridoma technique (see Kozbor et al., 1983, Immunol. Today 4:72), the EBV-hybridoma technique (see Cole et al., pp. 77-96 In Monoclonal Antibodies and Cancer Therapy, Alan R. Liss, Inc., 1985) or trioma techniques. The technology for producing hybridomas is well known (see generally Current Protocols in Immunology, Coligan et al. ed., John Wiley & Sons, New York, 1994). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody-secreting hybridomas, a monoclonal antibody directed against a protein of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

The invention also provides recombinant antibodies that specifically bind a protein of the invention. In preferred embodiments, the recombinant antibodies specifically binds an S. saprophyticus protein or fragment thereof. Recombinant antibodies include, but are not limited to, chimeric and humanized monoclonal antibodies, comprising both human and non-human portions, single-chain antibodies and multi-specific antibodies. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.) Single-chain antibodies have an antigen binding site and consist of a single polypeptide. They can be produced by techniques known in the art, for example using methods described in Ladner et. al U.S. Pat. No. 4,946,778 (which is incorporated herein by reference in its entirety); Bird et al., (1988) Science 242:423-426; Whitlow et al., (1991) Methods in Enzymology 2:1-9; Whitlow et al., (1991) Methods in Enzymology 2:97-105; and Huston et al., (1991) Methods in Enzymology Molecular Design and Modeling: Concepts and Applications 203:46-88. Multi-specific antibodies are antibody molecules having at least two antigen-binding sites that specifically bind different antigens. Such molecules can be produced by techniques known in the art, for example using methods described in Segal, U.S. Pat. No. 4,676,980 (the disclosure of which is incorporated herein by reference in its entirety); Holliger et al., (1993) Proc. Natl. Acad. Sci. USA 90:6444-6448; Whitlow et al., (1994) Protein Eng. 7:1017-1026 and U.S. Pat. No. 6,121,424.

Humanized antibodies are antibody molecules from non-human species having one or more complementarity determining regions (CDRs) from the non-human species and a framework region from a human immunoglobulin molecule. (See, e.g., Queen, U.S. Pat. No. 5,585,089, which is incorporated herein by reference in its entirety.) Humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

More particularly, humanized antibodies can be produced, for example, using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide corresponding to an S. saprophyticus polypeptide Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995) Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a murine antibody, is used to guide the selection of a completely human antibody recognizing the same epitope (Jespers et al., 1994, Bio/technology 12:899-903).

The antibodies of the invention can be isolated after production (e.g., from the blood or serum of the subject) or synthesis and further purified by well-known techniques. For example, IgG antibodies can be purified using protein A chromatography. Antibodies specific for a protein of the invention can be selected or (e.g., partially purified) or purified by, e.g., affinity chromatography. For example, a recombinantly expressed and purified (or partially purified) protein of the invention is produced as described herein, and covalently or non-covalently coupled to a solid support such as, for example, a chromatography column. The column can then be used to affinity purify antibodies specific for the proteins of the invention from a sample containing antibodies directed against a large number of different epitopes, thereby generating a substantially purified antibody composition, i.e., one that is substantially free of contaminating antibodies. By a substantially purified antibody composition is meant, in this context, that the antibody sample contains at most only 30% (by dry weight) of contaminating antibodies directed against epitopes other than those of the desired protein of the invention, and preferably at most 20%, yet more preferably at most 10%, and most preferably at most 5% (by dry weight) of the sample is contaminating antibodies. A purified antibody composition means that at least 99% of the antibodies in the composition are directed against the desired protein of the invention.

In a preferred embodiment, the substantially purified antibodies of the invention may specifically bind to a signal peptide, a secreted sequence, an extracellular domain, a transmembrane or a cytoplasmic domain of a protein of the invention. In a particularly preferred embodiment, the substantially purified antibodies of the invention specifically bind to a secreted sequence or an extracellular domain of the amino acid sequences of a protein of the invention. In a more preferred embodiment, the substantially purified antibodies of the invention specifically bind to a secreted sequence or an extracellular domain of the amino acid sequences of a marker protein.

An antibody directed against a protein of the invention can be used to isolate the protein by standard techniques, such as affinity chromatography or immunoprecipitation. Moreover, such an antibody can be used to detect an S. saprophyticus protein or fragment thereof (e.g., in a cellular lysate or cell supernatant) in order to evaluate the level and pattern of expression of the marker. The antibodies can also be used diagnostically to monitor protein levels in tissues or body fluids (e.g. in a cervical-associated body fluid) as part of a clinical testing procedure, e.g., to, for example, determine the efficacy of a given treatment regimen. Detection can be facilitated by the use of an antibody derivative, which comprises an antibody of the invention coupled to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include 125I, 131I, 35S or 3H.

Antibodies of the invention may also be used as therapeutic agents in treating an S. saprophyticus-associated disease and disorder. In a preferred embodiment, completely human antibodies of the invention are used for therapeutic treatment of human patients, particularly those having an S. saprophyticus-associated disease and disorder, e.g., urinary tract infection.

Accordingly, in one aspect, the invention provides substantially purified antibodies, antibody fragments and derivatives, all of which specifically bind to a protein of the invention and preferably, an S. saprophyticus protein. In various embodiments, the substantially purified antibodies of the invention, or fragments or derivatives thereof, can be human, non-human, chimeric and/or humanized antibodies. In another aspect, the invention provides non-human antibodies, antibody fragments and derivatives, all of which specifically bind to a protein of the invention and preferably, a marker protein. Such non-human antibodies can be goat, mouse, sheep, horse, chicken, rabbit, or rat antibodies. Alternatively, the non-human antibodies of the invention can be chimeric and/or humanized antibodies. In addition, the non-human antibodies of the invention can be polyclonal antibodies or monoclonal antibodies. In still a further aspect, the invention provides monoclonal antibodies, antibody fragments and derivatives, all of which specifically bind to a protein of the invention and preferably, an S. saprophyticus protein. The monoclonal antibodies can be human, humanized, chimeric and/or non-human antibodies.

XI. Kits Containing Nucleic Acids. Polypeptides or Antibodies of the Invention

The nucleic acid, polypeptides and antibodies of the invention can be combined with other reagents and articles to form kits. Kits for diagnostic purposes typically comprise the nucleic acid, polypeptides or antibodies in vials or other suitable vessels. Kits typically comprise other reagents for performing hybridization reactions, polymerase chain reactions (PCR), or for reconstitution of lyophilized components, such as aqueous media, salts, buffers, and the like. Kits may also comprise reagents for sample processing such as detergents, chaotropic salts and the like. Kits may also comprise immobilization means such as particles, supports, wells, dipsticks and the like. Kits may also comprise labeling means such as dyes, developing reagents, radioisotopes, fluorescent agents, luminescent or chemiluminescent agents, enzymes, intercalating agents and the like. With the nucleic acid and amino acid sequence information provided herein, individuals skilled in art can readily assemble kits to serve their particular purpose. Kits further can include instructions for use.

XII. Pharmaceutical Compositions

The nucleic acid molecules e.g., antisense nucleic acid molecules, proteins, protein fragments, small molecules, and anti-S. saprophyticus antibodies (also referred to herein as “active compounds”) of the invention can be incorporated into pharmaceutical compositions suitable for administration. Such compositions typically comprise the nucleic acid molecule, polypeptide, small molecules, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral, inhalation, transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a fragment of an S. saprophyticus polypeptide or an anti-S. saprophyticus antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the drug metabolism and pharmacokinetic studies, e.g., cell culture assays and animal studies, can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of activity) and the minimal inhibitory concentration (MIC) as determined in cell culture. Such information can be used to more accurately determine useful doses in humans. Levels in plasma may be measured, for example, by high performance liquid chromatography.

As defined herein, a therapeutically effective amount of polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 0.001 to 10 mg/kg, 0.01 to 9 mg/kg, 0.1 to 8 mg/kg, 1 to 7 mg/kg, or 2 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a polypeptide or antibody can include a single treatment or, preferably, can include a series of treatments.

In a preferred example, a subject is treated with antibody or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e.,. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

XIII. Methods of Treatment

The present invention provides for both prophylactic and therapeutic methods of treating a subject at risk of (or susceptible to) a disorder or having a disorder associated with aberrant or unwanted S. saprophyticus nucleic acid expression or polypeptide activity.

With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the invention provides methods for tailoring an individual's prophylactic or therapeutic treatment with either the S. saprophyticus polypeptide molecules of the present invention or S. saprophyticus polypeptide modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

(A) Prophylactic Methods

In one aspect, the invention provides a method for preventing in a subject, a disease or condition associated with an aberrant or unwanted S. saprophyticus nucleic acid expression or polypeptide activity, by administering to the subject an agent which modulates S. saprophyticus nucleic acid expression or at least one S. saprophyticus polypeptide activity. Subjects at risk for a disease which is associated with, caused or contributed to infection by S. saprophyticus can be identified by, for example, any or a combination of diagnostic or prognostic assays as described herein. Administration of a prophylactic agent can occur prior to the manifestation of symptoms characteristic of the S. saprophyticus-associated disease or disorder, such that a disease or disorder is prevented or, alternatively, delayed in its progression. For example, an S. saprophyticus polypeptide agonist or an S. saprophyticus polypeptide antagonist agent can be used for treating the subject. The appropriate agent can be determined based on screening assays described herein.

(B) Therapeutic Methods

Another aspect of the invention pertains to methods of modulating S. saprophyticus nucleic acid expression or polypeptide activity for therapeutic purposes, e.g., to treat an S. saprophyticus-associated disease or disorder, e.g., to reduce or ameliorate S. saprophyticus infection in a subject. Accordingly, in an exemplary embodiment, the modulatory method of the invention involves contacting a bacterial cell expressing an S. saprophyticus polypeptide or a host tissue which is infected with S. saprophyticus with an agent that modulates one or more of the activities of an S. saprophyticus polypeptide activity, such that an S. saprophyticus polypeptide activity is modulated or S. saprophyticus bacteria is killed, or there is a reduction in bacterial growth, e.g., either by the host immune system or by direct action of the agent. An agent that modulates an S. saprophyticus activity can be an agent as described herein, such as, an S. saprophyticus antibody, an S. saprophyticus polypeptide agonist or antagonist, a peptidomimetic of an S. saprophyticus polypeptide agonist or antagonist, vaccine, or other small molecule. In one embodiment, the agent stimulates one or more S. saprophyticus polypeptide activities. Examples of such stimulatory agents include active S. saprophyticus polypeptide and a nucleic acid molecule encoding S. saprophyticus that has been introduced into the cell. In another embodiment, the agent inhibits one or more S. saprophyticus polypeptide activities. Examples of such inhibitory agents include antisense S. saprophyticus nucleic acid molecules, anti-S. saprophyticus antibodies, small molecules, and S. saprophyticus polypeptide inhibitors. These modulatory methods can be performed in vivo (e.g., by administering the agent to a subject). As such, the present invention provides methods of treating an individual afflicted with a to treat an S. saprophyticus-associated disease or disorder, e.g., to reduce or ameliorate S. saprophyticus infection in a subject. In one embodiment, the method involves administering an agent (e.g., an agent identified by a screening assay described herein), or combination of agents that modulates (e.g., upregulates or downregulates) S. saprophyticus nucleic acid expression or polypeptide activity.

This invention is further illustrated by the following examples which should not be construed as limiting. Furthermore, all references, Accession Numbers, sequences, patent applications, patents, and published patent applications, cited throughout this application are hereby incorporated by reference.

### EXAMPLES

### Example 1

### Experimental Design and Methods

Random Sequencing Strategy and Library Construction.

The overall approach to sequencing bacterial genomes has been well established since first described by Fleischmann et al. (Science 269:449-604, 1995). The theory of the approach is centered behind the Lander and Waterman (Genomics 2:231-239, 1988) hypothesis of a Poisson distribution that describes the probability that any given base will not be sequenced after a certain amount of random sequence has been generated. Typically, five to six fold coverage of a genome sequenced randomly yields<1% of the total genome unrepresented.

Chromosomal DNA from a recent clinical isolate of Staphylococcus saprophyticus (given the strain name of ARC 1259) was prepared from a fresh overnight culture that had been incubated at 37° C. on blood agar plates. The strain had been purified from a single colony to ensure purity. Bacterial colonies were collected and processed using a commercial genomic DNA isolation kit (Promega). The precipitated chromosomal DNA was washed extensively with ethanol to remove contaminating salts. Two libraries containing random DNA fragments of S. saprophyticus ARC 1259 were generated for the random sequencing phase. These libraries were constructed with different average size random inserts in order to help anchor the subsequent sequences. The first library contained inserts averaging 1,500 base pairs, while the inserts of the second library averaged 3,000 base pairs. The plasmid DNA from representative colonies of these libraries was produced and sequenced according to routine molecular biological protocols. For both libraries, approximately 1,000 reactions were initially run and examined to ensure that the libraries contained random inserts as expected. A total of 22,351 reactions were run to generate the random sequence, and these reactions averaged 771 base pairs per reaction. With a predicted genome size of 2.4 Mb for S. saprophyticus this represented a 6.4 fold coverage.

Assembly and Informatics

Base calls and quality scores were determined using the program PHRED (Ewing et al., Genome Res. 8: 175-185, 1998; Ewing and Green, Genome Res. 8: 685-734, 1998). Reads were assembled using PHRAP (P. Green, Abstracts of DOE Human Genome Program Contractor-Grantee Workshop V, January 1996, p. 157) with default program parameters and quality scores and viewed and corrected using Consed (Gordon et al., Genome Res. 8:195-202, 1998). After the initial assembly, the random sequences were condensed into 247 independent contigs. An initial attempt to order these contigs and close some of the physical gaps was performed. The terminal 1,000 base pairs from each end of every contig were used to search non-redundant protein databases to identify the presence of orthologous peptide sequences using the BLAST algorithm (Altschul et al., J. Mol. Biol. 215:403-410, 1990). If two independent contigs contained fragments of the same protein sequence from the database in a suitable way, then it was suggestive that these contigs were linked. To determine the intervening sequence, oligonucleotide primers were designed from the contig ends to amplify a DNA product using the polymerase chain reaction (PCR) from S. saprophyticus ARC 1259 genomic DNA. The reaction conditions were as follows: 50 ng of genomic DNA, 3.2 pmol of each primer and 48 ul of diluted High-Fidelity PCR Master Mix (Roche) were combined in a 96-well plate. Thirty cycles consisting of 60 seconds at 94° C. for denaturation, 60 seconds at 52° C. for primer annealing and 3 minutes at 72° C. for extension were carried out in a 96-well plate on a Perkin Elmer GeneAmp 9600 machine. The resulting PCR products were purified using the PCR Clean-up Kit (EDGE Biosystems) and sequenced using the amplification primers on an ABI 3100 DNA sequencer using Big Dye Terminator Cycle sequencing Kit (v.3.1). These sequences, which closed the physical gaps between specific contigs, were then incorporated into the PHRAP-based assembly.

Once the number of contigs had been reduced, putative open reading frames (ORFs) were identified. Stretches of DNA that possessed a suitable translational intitiation codon (AUG, GUG or CUG) and did not contain a translational termination codon for a minimum of 33 triplet codons were identified and called putative ORFs. The predicted protein sequences of these putative ORFs were used to search non-redundant protein databases to identify whether they displayed any homology to proteins from other bacterial species. All the ORFs and their homology searches were then visually inspected.

### Example 2

### Construction and Operation of DNA Microarrays

The sequences of the invention may additionally be used in the construction and application of DNA microarrays (the design, methodology, and uses of DNA arrays are well known in the art, and are described, for example, in Schena, M. et al. (1995) Science 270: 467-470; Wodicka, L. et al. (1997) Nature Biotechnology 15: 1359-1367; DeSaizieu, A. et al. (1998) Nature Biotechnology 16: 45-48; and DeRisi, J. L. et al. (1997) Science 278: 680-686).

DNA microarrays are solid or flexible supports consisting of nitrocellulose, nylon, glass, silicone, or other materials. Nucleic acid molecules may be attached to the surface in an ordered manner. After appropriate labeling, other nucleic acids or nucleic acid mixtures can be hybridized to the immobilized nucleic acid molecules, and the label may be used to monitor and measure the individual signal intensities of the hybridized molecules at defined regions. This methodology allows the simultaneous quantification of the relative or absolute amount of all or selected nucleic acids in the applied nucleic acid sample or mixture. DNA microarrays, therefore, permit an analysis of the expression of multiple (as many as 6800 or more) nucleic acids in parallel (see, e.g., Schena, M. (1996) BioEssays 18(5): 427-431).

The sequences of the invention may be used to design oligonucleotide primers which are able to amplify defined regions of one or more S. saprophyticus genes by a nucleic acid amplification reaction such as the polymerase chain reaction. The choice and design of the 5′ or 3′ oligonucleotide primers or of appropriate linkers allows the covalent attachment of the resulting PCR products to the surface of a support medium described above (and also described, for example, Schena, M. et al. (1995) Science 270: 467-470).

Nucleic acid microarrays may also be constructed by in situ oligonucleotide synthesis as described by Wodicka, L. et al. (1997) Nature Biotechnology 15: 1359-1367. By photolithographic methods, precisely defined regions of the matrix are exposed to light. Protective groups which are photolabile are thereby activated and undergo nucleotide addition, whereas regions that are masked from light do not undergo any modification. Subsequent cycles of protection and light activation permit the synthesis of different oligonucleotides at defined positions. Small, defined regions of the genes of the invention may be synthesized on microarrays by solid phase oligonucleotide synthesis.

The nucleic acid molecules of the invention present in a sample or mixture of nucleotides may be hybridized to the microarrays. Alternatively, fragments of the nucleic acid molecules of the invention, e.g., oligonucleotides of about, for example, 40-100 nucleotides from each of the gene or segment of a gene of the invention may be hybridized to the microarrays. The nucleic acid molecules can be labeled according to standard methods. In brief, nucleic acid molecules (e.g., RNA, including, but not limited to, mRNA or rRNA molecules or DNA molecules, including, but not limited to, cDNA molecules) are labeled by the incorporation of isotopically or fluorescently labeled nucleotides, e.g., during reverse transcription or DNA synthesis. Hybridization of labeled nucleic acids to microarrays is described (e.g., in Schena, M. et al. (1995) supra; Wodicka, L. et al. (1997), supra; and DeSaizieu A. et al. (1998), supra). The detection and quantification of the hybridized molecule are tailored to the specific incorporated label. Radioactive labels can be detected, for example, as described in Schena, M. et al. (1995) supra) and fluorescent labels may be detected, for example, by the method of Shalon et al. (1996) Genome Research 6: 639-645).

The application of the sequences of the invention to DNA microarray technology, as described above, permits comparative analyses of different strains of S. saprophyticus. For example, studies of inter-strain variations based on individual transcript profiles and the identification of genes that are important for specific and/or desired strain properties such as pathogenicity are facilitated by nucleic acid array methodologies. The DNA microarrays described herein may also be used to determine or confirm mechanism of action of an inhibitor, e.g., a small molecule inhibitor, or to identify an inhibitor, e.g., a small molecule inhibitor, of S. saprophyticus nucleic acid expression or polypeptide activity.

### Example 3

### Analysis of the Dynamics of Cellular Protein Populations (Proteomics)

The genes, compositions, and methods of the invention may be applied to study the interactions and dynamics of populations of proteins, termed ‘proteomics’. Protein populations of interest include, but are not limited to, the total protein population of S. saprophyticus (e.g., in comparison with the protein populations of other organisms), those proteins which are active under specific environmental or metabolic conditions, or those proteins which are active during specific phases of growth and development.

Protein populations can be analyzed by various well-known techniques, such as gel electrophoresis. Cellular proteins may be obtained, for example, by lysis or extraction, and may be separated from one another using a variety of electrophoretic techniques. Sodium dodecyl sulfate polyacrylamide gel electrophoresis (SDS-PAGE) separates proteins largely on the basis of their molecular weight. Isoelectric focusing polyacrylamide gel electrophoresis (IEF-PAGE) separates proteins by their isoelectric point (which reflects not only the amino acid sequence but also posttranslational modifications of the protein). Another, more preferred method of protein analysis is the consecutive combination of both IEF-PAGE and SDS-PAGE, known as 2-D-gel electrophoresis (described, for example, in Hermann et al. (1998) Electrophoresis 19: 3217-3221; Fountoulakis et al. (1998) Electrophoresis 19: 1193-1202; Langen et al. (1997) Electrophoresis 18: 1184-1192; Antelmann et al. (1997) Electrophoresis 18: 1451-1463). Other separation techniques may also be utilized for protein separation, such as capillary gel electrophoresis; such techniques are well known in the art.

Proteins separated by these methodologies can be visualized by standard techniques, such as by staining or labeling. Suitable stains are known in the art, and include Coomassie Brilliant Blue, silver stain, or fluorescent dyes such as Sypro Ruby (Molecular Probes). The inclusion of radioactively labeled amino acids or other protein precursors (e.g., ³⁵S-methionine, ³⁵S-cysteine, ¹⁴C-labelled amino acids, ¹⁵N-amino acids, ¹⁵NO₃ or ¹⁵NH₄⁺ or ¹³C-labelled amino acids) in the medium of S. saprophyticus permits the labeling of proteins from these cells prior to their separation. Similarly, fluorescent labels may be employed. These labeled proteins can be extracted, isolated and separated according to the previously described techniques.

Proteins visualized by these techniques can be further analyzed by measuring the amount of dye or label used. The amount of a given protein can be determined quantitatively using, for example, optical methods and can be compared to the amount of other proteins in the same gel or in other gels. Comparisons of proteins on gels can be made, for example, by optical comparison, by spectroscopy, by image scanning and analysis of gels, or through the use of photographic films and screens. Such techniques are well-known in the art.

To determine the identity of any given protein, direct sequencing or other standard techniques may be employed. For example, N- and/or C-terminal amino acid sequencing (such as Edman degradation) may be used, as may mass spectrometry (in particular MALDI or ESI techniques (see, e.g., Langen et al. (1997) Electrophoresis 18: 1184-1192)). The protein sequences provided herein can be used for the identification of S. saprophyticus proteins by these techniques.

The information obtained by these methods can be used to compare patterns of protein presence, activity, or modification between different samples from various biological conditions.

Equivalents

Those of ordinary skill in the art will recognize, or will be able to ascertain using no more than routine experimentation, many equivalents to the specific embodiments of the invention described herein. Such equivalents are intended to be encompassed by the following claims.

1. An isolated nucleic acid molecule comprising a nucleotide sequence as set forth in FIG. 1, or a fragment thereof.

2. An isolated nucleic acid molecule which encodes a polypeptide sequence selected from the group consisting of those sequences set forth in FIG. 1, or a fragment thereof.

3. An isolated nucleic acid molecule which encodes a naturally occurring allelic variant of a polypeptide selected from the group of amino acid sequences consisting of those sequences set forth in FIG. 1.

4. An isolated nucleic acid molecule comprising a nucleotide sequence which is at least 80% homologous to a nucleotide sequence selected from the group consisting of those sequences set forth in FIG. 1, or a portion thereof.

5. An isolated nucleic acid molecule comprising a fragment of at least 15 nucleotides of a nucleic acid comprising a nucleotide sequence selected from the group consisting of those sequences set forth in FIG. 1.

6. A vector comprising the nucleic acid molecule of claim 1.

7. The vector of claim 6, which is an expression vector.

8. A host cell transfected with the expression vector of claim 7.

9. The host cell of claim 8, wherein said cell is a microorganism.

10. The host cell of claim 8, wherein said cell belongs to the genus Staphylococcus or Escherichia.

11. A method of producing a polypeptide comprising culturing the host cell of claim 8 in an appropriate culture medium to, thereby, produce the polypeptide, or fragment thereof.

12. A method for identifying a compound which modulates the activity of a polypeptide encoded by the nucleic acid of claim 1 comprising:
a) contacting a polypeptide encoded by the nucleic acid of claim 1 with a test compound; and
b) determining the effect of the test compound on the activity of the polypeptide to thereby identify a compound which modulates the activity of the polypeptide.

13. The method of claim 12, wherein said compound is a small molecule.

14. A method for diagnosing the presence or activity of S. saprophyticus in a subject, comprising detecting the presence of one or more of the sequences set forth in FIG. 1, thereby diagnosing the presence or activity of S. saprophyticus in the subject.

15. A vaccine formulation for prophylactic or therapeutic treatment of an S. saprophyticus infection comprising an effective amount of at least one isolated nucleic acid molecule of claim 1.

16. A vaccine formulation for prophylactic or therapeutic treatment of an S. saprophyticus infection comprising an effective amount of at least one S. saprophyticus polypeptide encoded by a nucleic acid of claim 1.

17. A vaccine formulation of claim 15, further comprising a pharmaceutically acceptable carrier.
